(19) United States
(12) Patent Application Publication
(10) Pub. No.: US 2023/0329630 A1
(43) Pub. Date: Oct. 19, 2023

(54) COMPUTERIZED DECISION SUPPORT TOOL AND MEDICAL DEVICE FOR RESPIRATORY CONDITION MONITORING AND CARE

(71) Applicant: PFIZER INC., New York, NY (US)

(72) Inventors: Shyamal Patel, Melrose, MA (US); Paul William Wacnik, Brookline, MA (US); Kara Chappie, Cambridge, MA (US); Robert Mather, Cambridge, MA (US); Brian Tracey, Arlington, MA (US); Maria del Mar Santamaria Serra, Cambridge, MA (US)

(73) Assignee: PFIZER INC., New York, NY (US)

(21) Appl. No.: 18/043,271

(22) PCT Filed: Aug. 30, 2021
(86) PCT No.: PCT/US2021/048242

§ 371 Date: Feb. 27, 2023

Related U.S. Application Data
(60) Provisional application No. 63/071,718, filed on Aug. 28, 2020.
(60) Provisional application No. 63/238,103, filed on Aug. 27, 2021.

(51) Int. Cl. A61B 5/00; A61B 5/08; A61B 7/00; A61K 31/675; A61K 38/06; G10L 15/02; G10L 25/66; G10L 15/26; G10L 15/22; G16H 10/60; G16H 40/20; G16H 20/10; G16H 50/30

(52) U.S. Cl. CPC A61B 5/4803; A61B 5/08; A61B 5/7275; A61B 5/4839; A61B 5/4848; A61B 5/4842; A61B 7/003; A61B 5/7278; A61K 31/675; A61K 38/06; G10L 15/02; G10L 25/66; G10L 15/26; G10L 15/22; G16H 10/60; G16H 40/20; G16H 20/10; G16H 50/30; G10L 2015/025

(57) ABSTRACT

Technology is disclosed for monitoring a user's respirator), condition and provide decision support by analyzing a user's audio data. Spoken phonemes may be detected within audio data, and acoustic features may be extracted for the phonemes. A distance metric may be computed to compare phoneme feature sets of a user. Based on the comparison, a determination about the user's respiratory condition, such as whether the user has a respiratory condition (e.g., an infection) and/or whether the condition is changing, may be made. Some aspects include predicting the user's respiratory condition in the future utilizing the phoneme feature sets. Decision support tools in the form of computer applications or services may utilize the detected or predicted respiratory condition information to initiate an action for treating a current condition or mitigating a future risk.

### BACKGROUND OF THE INVENTION

Viral and bacterial respiratory infections, such as influenza, impact a large population every year and have symptoms that range from minimal to severe. Typically, viral or bacterial levels peak in the body of an infected person ahead of self-reported symptoms, often leaving an individual unaware about the infection. Additionally, most individuals typically find it difficult to detect new or mild respiratory symptoms or to quantify any change in symptoms (either when symptoms worsen or improve). However, early detection of respiratory infections may lead to a more effective intervention that reduces the duration and/or severity of the infection. Additionally, early detection is beneficial in clinical trials, since if it is too late such that the infectious agent load in a potential trial participant drops too low, it may not be possible to confirm potential participant's symptoms correlated to the infection of interest. Accordingly, there is a need for tools utilizing objective measures to detect and monitor respiratory infection symptoms, prior to the symptoms rising to a level typically required to prompt a visit to a healthcare provider.

### SUMMARY OF THE INVENTION

This summary is provided to introduce a selection of concepts in a simplified form that is further described below in the detailed description. This summary is neither intended to identify key features or essential features of the claimed subject matter nor to be used in isolation as an aid in determining the scope of the claimed subject matter.

Embodiments of the technologies described in the present disclosure enable improved computerized decision support tools for monitoring an individual's respiratory condition, such as by determining and quantifying changes occurring to the individual's respiratory condition, determining a likelihood of the individual having a respiratory condition (which may be a respiratory infection), or predicting the individual's respiratory condition in the future.

At a high level, these embodiments may include utilizing audio data acquired by a sensor device, such as a microphone, which may be integrated into a user computing device, such as a smartphone, to automatically detect data indicating the individual's respiratory condition. For example, audio data may be provided, by a user of an embodiment of these technologies, as audio samples, which may be in the form of a sustained phonation (e.g., “aaaaaaaa”), scripted speech, or unscripted speech acquired during casual interactions with a computing device (e.g., a smart speaker). Some embodiments may also provide instructions to guide a user through a procedure for providing audio data usable for monitoring the user's respiratory condition. In this way, data for monitoring a respiratory condition may be obtained reliably in a non-laboratory setting and in an unobtrusive manner while the user is carrying out everyday activities, including that in a user's home. Accordingly, the embodiments described herein increase the likelihood of user compliance while still providing reliable data to accurately and effectively monitor the user's respiratory condition.

According to an embodiment, phonemes may be detected from recorded audio data of a user, and acoustic features for the detected phonemes may be extracted or determined. These features may comprise a phoneme feature set or a feature vector that characterizes a user's respiratory condition at a particular time interval (e.g., date-time) and thus may be considered to be associated with that particular time interval. The user may provide multiple audio voice samples at multiple time intervals (e.g., each day, or each morning and evening for multiple days), such that each determined phoneme feature set is associated with a particular time interval at which time the audio sample data was provided by the user. For example, in one aspect, the detected phonemes may comprise /a/, /e/, /m/, or /n/, or any combination thereof. In another aspect, the detected phonemes may comprise one or more of the cardinal vowel phonemes, such as /i/, /e/, /ε/, /a/, /α/, / [symbol] /, /o/, and/u/, and may further comprise the phonemes /n/ and/or /m/. The detected phoneme may be utilized by an embodiment of the technologies described herein to determine a biomarker for respiratory condition. In another aspect, a combination of one or more of these phonemes or their features may be utilized to determine a biomarker. In still another aspect, other phonemes or phoneme features and/or respiratory or voice related data may be utilized to determine a biomarker.

Phoneme feature sets for different time intervals may be compared to determine differences between the values of the phoneme features. For instance, a Euclidian distance measurement may be determined between the phoneme feature sets. Similarly, in some embodiments, a Levenshtein distance may be determined, such as for implementations comparing the user reading aloud a passage. Based on differences between phoneme feature sets from different time intervals, a determination may be provided about the user's respiratory condition. For example, an embodiment of this disclosure may determine that the user generally has a respiratory condition, that the user has a specific type of respiratory condition (e.g., influenza), and/or that the user's respiratory condition is worsening, improving, and/or not changing over a time period. In this way, the technologies disclosed herein may be utilized to automatically provide a determination regarding a user's respiratory condition, such as a likelihood of respiratory infection, based on objective data of the user's respiratory condition, such as quantifiable detected changes in phoneme features. In some embodiments, these determined differences between the phoneme features may be utilized to predict a user's future respiratory condition (i.e., at a future time). In some embodiments, contextual information, such as a user's physiological data, self-reporting symptoms, sleep data, location, and/or weather-related information, may also be utilized in conjunction with the phoneme features data to determine or forecast a user's respiratory condition.

Based on the determination of the user's respiratory condition, a computing device may initiate an action. By way of example and without the limitation, the action may include electronically communicating an alert or a notification to the user, a clinician or a caregiver for the user. The notification may include information about the user's respiratory condition, and in some instances may include a detected change in the user's respiratory condition and/or a forecast of the user's respiratory condition in the future. Another example of an action may comprise communicating a recommendation for treatment or support based on the user's determined or forecasted respiratory condition. For example, the recommendation may comprise consulting with a healthcare provider, continuing an existing prescription or over-the-counter medicine (such as re-fill a prescription), modifying a dosage or medication of a current treatment protocol, and/or to continue monitoring the respiratory condition. In some aspects, the action may include initiating one or more of these or other recommendations, such as automatically scheduling an appointment with the user's healthcare provider and/or communicating a notification to a pharmacy for re-filling a prescription.

In some instances, utilizing the acoustic feature information from user's voice samples, a respiratory condition may be determined (e.g., the user likely has an infection) even if the user does not feel symptomatic. This capability, as provided by some embodiments of the technologies disclosed herein, is an advantage and improvement over conventional technologies, which may rely on subjective or objective data only, acquired from a visit to a clinician after onset of symptoms. This early detection and warning of a respiratory condition may enable more effective treatment to reduce the duration and/or severity of the condition. Further, these embodiments enabling early detection may be particularly useful for combatting respiratory-based pandemics, such as severe acute respiratory syndrome coronavirus 2 (SARS-CoV-2) or coronavirus disease (COVID-19), by providing an early warning of respiratory condition than conventional approaches. Where the condition is caused by a virus or bacteria, the early warning may enable the user to take precautions against transmission sooner (e.g., wear a mask, self-quarantine, or practice social distancing) and, therefore, reduce the likelihood of transmission to others. Early detection provided through embodiments of this disclosure may also be useful in clinical trials studying vaccines and/or treatment of respiratory infections. Some embodiments may enable participants to have a confirmation regarding any symptoms being correlated to the infection of interest before the infectious agent load drops too low, which is a frequently occurring problem with the conventional technologies used in clinical trials.

Further, utilizing acoustic features from voice recordings to monitor respiratory condition enable improved accuracy in treating individuals with respiratory conditions. For example, a potential respiratory condition of the individual may be tracked at home in accordance with this disclosure utilizing the voice recordings to more precisely determine when treatment, such as an antibiotic, is needed rather than prescribing treatment to an individual prematurely and/or for too long a time period. Further, tracking the progression of the condition of the individual, who is being treated in accordance with embodiments of this disclosure, may help in determining whether a change in treatment, such as changing medication and/or dosage, is recommended or not. In this way, the technologies disclosed herein may facilitate more precise utilization of antibiotics/anti-microbial medicines, since such medicines need to be prescribed or continued based on an objective quantifiable change detected in an individual's respiratory condition.

### BRIEF DESCRIPTION OF THE DRAWING

Aspects of the disclosure are described in detail below with reference to the attached figures, wherein:

FIG. 1 is a block diagram of an example operating environment suitable for implementing aspects of the present disclosure;

FIG. 2 is a diagram depicting an example computing architecture suitable for implementing aspects of the present disclosure;

FIG. 3A illustratively depicts a diagrammatic representation of an example process for monitoring respiratory conditions, in accordance with an embodiment of the present disclosure;

FIG. 3B illustratively depicts a diagrammatic representation of an example process of collecting data for monitoring respiratory conditions, in accordance with an embodiment of the present disclosure;

FIGS. 4A-4F illustratively depict example scenarios utilizing various embodiments of the present disclosure;

FIGS. 5A-5E illustratively depict exemplary screenshots from a computing device showing aspects of example graphical user interfaces (GUIs), in accordance with various embodiments of the present disclosure;

FIG. 6A illustratively depicts a flow diagram of an example method for monitoring respiratory conditions, in accordance with an embodiment of the present disclosure;

FIG. 6B illustratively depicts a flow diagram of an example method for monitoring respiratory conditions, in accordance with another embodiment of the present disclosure;

FIG. 7 illustratively depicts representations of changes in example acoustic features over time, in accordance with an embodiment of the present disclosure;

FIG. 8 illustratively depicts a graphic representation of decay constants for respiratory infection symptoms, in accordance with an embodiment of the present disclosure;

FIG. 9 illustratively depicts a graphic representation of correlations between acoustic features and respiratory infection symptoms, in accordance with an embodiment of the present disclosure;

FIG. 10 illustratively depicts a graphic representation of the change in self-reported symptom scores over time for example individuals, in accordance with an embodiment of the present disclosure;

FIGS. 11A-11B illustratively depict graphic representations of rank correlation between distance metric computed for different acoustic features and self-reported symptom scores, in accordance with an embodiment of the present disclosure;

FIG. 12A illustratively depicts a graph representation of rank correlations between distance metrics and self-reported symptom scores across different individuals, in accordance with an embodiment of the present disclosure;

FIG. 12B illustratively depicts a statistically significant correlations between acoustic feature types and phonemes in accordance with an embodiment of the present disclosure;

FIG. 13 illustratively depicts graphic representations of relative changes in acoustic features and self-reported symptoms over time for three example individuals, in accordance with an embodiment of the present disclosure;

FIG. 14 illustratively depicts example representations of performance of a respiratory infection detector, in accordance with an embodiment of the present disclosure;

FIGS. 15A-15M depict an example computer program routine for extracting acoustic features for monitoring respiratory conditions, in accordance with various embodiments of the present disclosure; and

FIG. 16 is a block diagram of an exemplary computing environment suitable for use in implementing an embodiment of the present disclosure.

### DETAILED DESCRIPTION OF THE INVENTION

The subject matter of the present disclosure is described herein with specificity with the help of different aspects to meet statutory requirements. However, the description itself is not intended to limit the scope of this patent. The claimed subject matter might be embodied in other ways, to include different steps or combinations of steps similar to the ones described in this present disclosure, in conjunction with other present or future technologies. Moreover, although the terms “step” and/or “block” may be used herein to connote different elements of methods employed, the terms should not be interpreted as implying any particular order among or between various steps disclosed herein, unless and except when the order of individual steps is explicitly stated. Each method described herein may comprise a computing process that may be performed using any combination of a hardware, firmware, and/or software. For instance, various functions may be carried out by a processor executing instructions stored in a computer memory. The methods may also be embodied as computer-useable instructions stored on computer storage media. The methods may be provided by a stand-alone application, a service or a hosted service (stand-alone or in combination with another hosted service), or a plug-in to another product, to name a few.

Aspects of the present disclosure relate to computerized decision support tools for respiratory condition monitoring and care. Respiratory conditions impact a large population every year and have symptoms that range from minimal to severe. Such respiratory conditions may include respiratory infections caused by bacterial or viral agents such as influenza or may comprise non-infectious respiratory system symptoms. Although some aspects of this disclosure describe respiratory infections, it is contemplated that such aspects may apply respiratory condition generally.

Individuals typically find it difficult to detect new or mild respiratory symptoms, as well as to quantify change in symptoms (i.e., either when symptoms worsen or when they improve). Objective measures of respiratory condition are conventionally determined only when an individual sees a healthcare professional and a specimen analysis is performed. However, viral or bacterial levels that may cause a respiratory infection typically peak in the body of an infected individual ahead of self-reported symptoms, often leaving the individual unaware of the infection prior to receiving any diagnosis. For instance, individuals with influenza or coronavirus disease 2019 (COVID-19) may infect others prior to feeling symptoms. The inability to objectively measure mild symptoms of respiratory condition, such as an infection, at early stages increases the likelihood of transmission of an infection to other individuals, a longer duration of the respiratory condition, and a greater severity of the respiratory condition.

To improve monitoring and care of respiratory conditions, embodiments of the present disclosure may provide one or more decision support tools for determining a user's respiratory condition and/or forecasting the user's respiratory condition in the future based on acoustic data from user's voice recordings. For example, a user may provide audio data through voice recordings so that the acoustic features of phonemes (which may also be referred to herein as phoneme features) in the audio data may be determined. In on embodiment, a plurality of voice recordings may be received such that each recording corresponds to a different time interval (e.g., a voice recording may be obtained for each day over a series of days). Phoneme feature values from different time intervals may be compared to determine information about the user's respiratory condition, such as whether there has been a change in the user's respiratory condition over time or not. An action, such as an alert or decision support recommendation, may be automatically provided to the user and/or a clinician of the user based on the determination of the user's respiratory condition.

In one embodiment, and as further described herein, the acoustic information may be received from the monitored individual (which may be also referred herein as a user) by utilizing a sensor, such as a microphone. The acoustic information may comprise one or more recordings of the user's voice (e.g., vocalizations or other respiratory sounds). The voice recordings may include audio samples of a sustained phonation (e.g., “aaaaaaaa”), scripted speech, or unscripted speech, for example. The microphone may be integrated into or otherwise coupled to a user computing device, such as a smartphone, a smartwatch, or a smart speaker. In some instances, voice audio samples may be recorded at the user's home or during the user's everyday activities and may include data recorded during user's casual interactions with a smart speaker or other user computing device.

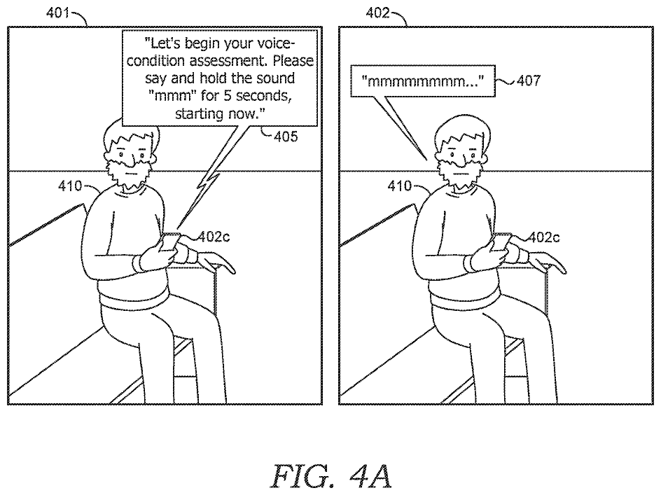

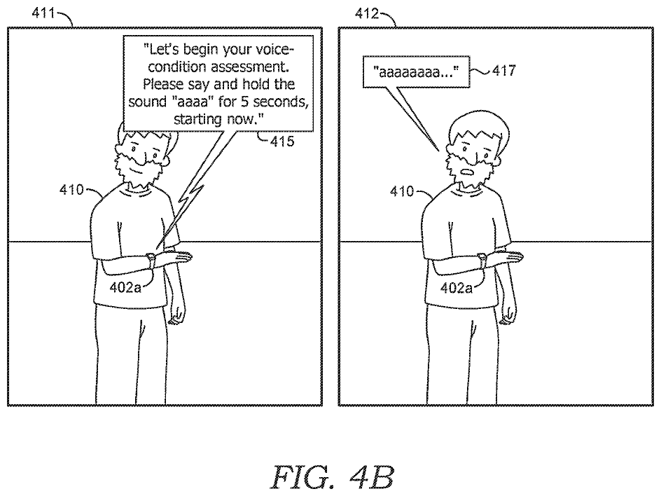

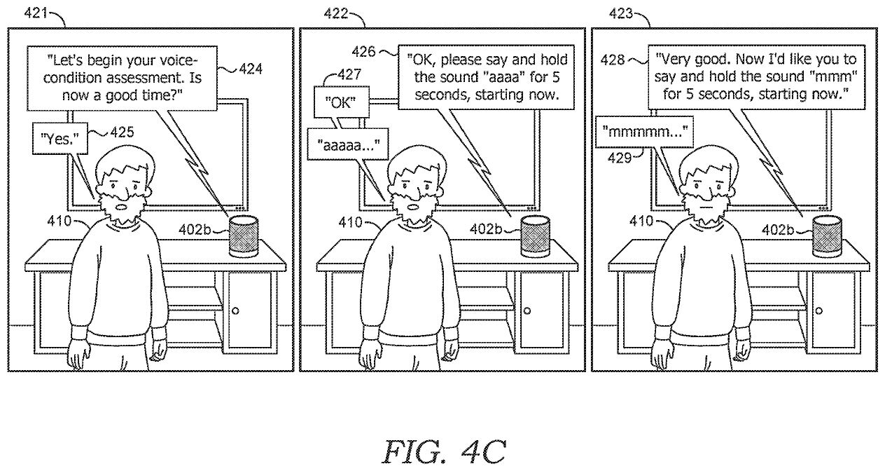

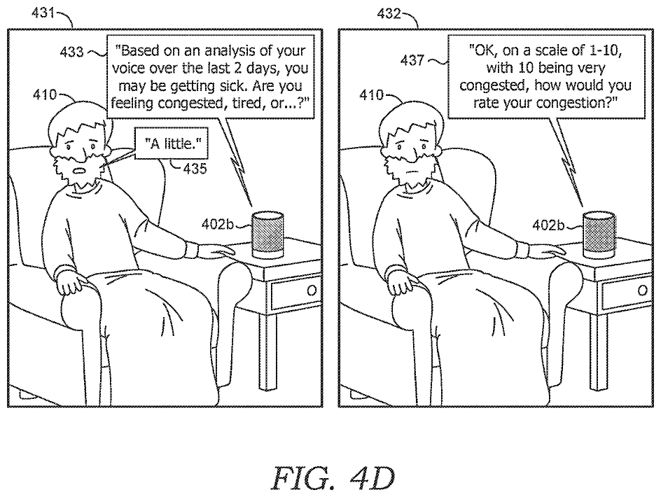

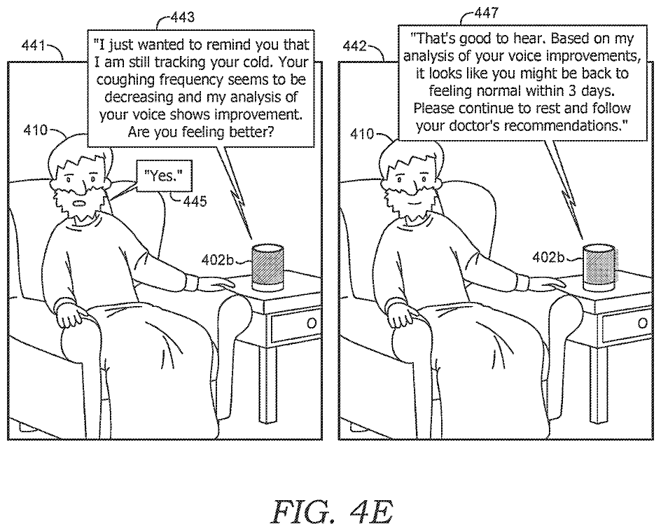

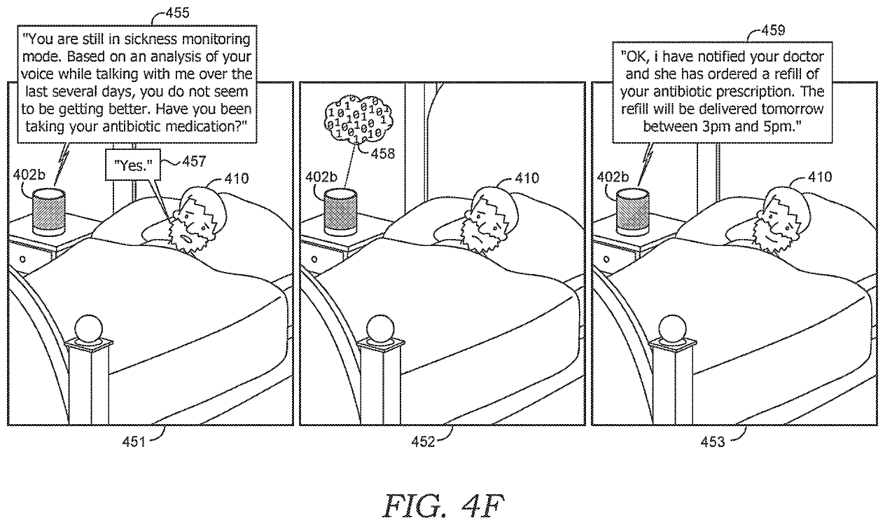

Some embodiments may also generate and/or provide instructions to guide a user through a procedure for providing audio data usable for monitoring the user's respiratory condition. For example, FIGS. 4A, 4B and 4C each show scenarios where a user computing device (or user device) is outputting instructions to a user (e.g., in the form of text and/or audible instructions) as part of an assessment exercise. The instructions may prompt the user to vocalize certain sounds and, in some embodiments, the duration for the vocalization (E.g., “Please say and hold the sound “aah” for five seconds.) In some embodiments, instructions may ask the user to hold or sustain a vocalization, such as a vocalization of one of the cardinal vowels such as /a/, for as long as the user is able. And in some embodiments, instructions include asking the user to read aloud a written passage. Some embodiments may further include providing the user with feedback to ensure the voice samples are useable, such as instructing the user when to start/stop, to speak longer, hold for a longer duration, reduce background noise, and/or other feedback for quality control.

In some embodiments, acoustic and voice information, such as phonemes, may be detected from the audio data received from the user. In one embodiment, the detected phonemes may include phonemes /a/, /m/, and /n/. In another embodiment, the detected phonemes include /a/, /e/, /m/, and /n/. In some embodiments of the technologies described herein, the detected phoneme may be utilized to determine a biomarker for respiratory condition detection and monitoring. Once phonemes are detected, acoustic features of the detected phonemes may be extracted or determined from the audio data. Examples of the acoustic features may include, without limitation, data characterizing measures of power and power variability, a pitch and a pitch variability, a spectral structure, and/or formants In some embodiments, different feature sets (i.e., different combinations of acoustic features) may be determined for different phonemes detected in the audio data. In an exemplary embodiment, 12 features are determined for the /n/ phoneme, 12 features are determined for the /m/ phoneme, and 8 features are determined for the /a/ phoneme. In some embodiments, pre-processing or signal condition operations may be performed to facilitate detecting phonemes and/or determining phoneme features. These operations may include, for example, trimming the audio sample data, frequency filtering, normalization, removing background noise, intermittent spikes, other acoustic artifacts, or other operations as described herein.

As audio data is acquired from the user over time, multiple phoneme feature sets, which may comprise phoneme feature vectors, may be generated and associated with different time intervals. In some embodiments, a time series may be assembled of successive phoneme feature sets for the user in chronological or reverse-chronological order, according to the time information associated with the feature sets. Differences or changes in the values of features within feature sets associated at different time instances or intervals may be determined. For example, differences in phoneme feature vectors for a user may be determined by comparing two or more phoneme feature vectors associated with different time instances or intervals. In one embodiment, the difference may be determined by computing a distance metric, such as a Euclidian distance between feature vectors. In some instances, one of the phoneme feature sets utilized for comparison represents a healthy baseline for the user. The healthy baseline feature set may be determined based on audio data acquired when the user is known or presumed to be without a respiratory condition. Similarly, a sick baseline feature set that is determined based on audio data acquired when the user is known or presumed to have a respiratory condition may be utilized.

Based on differences between phoneme feature sets from different times, information about the user's determination of the respiratory condition may be provided. In some embodiments, as further described herein, this determination may be provided as a respiratory-condition score. The respiratory-condition score may correspond to a likelihood or probability that the user has (or does not have) a respiratory condition such as an infection (e.g., either generally for any respiratory condition or for a particular respiratory condition). Alternatively, or in addition, a respiratory-condition score may indicate whether the user's respiratory condition is improving, worsening, or not changing. The example scenario of FIG. 4F, for instance, depicts an embodiment in which it is determined that a user is not recovering from a respiratory condition based on analysis of the user's voice information, as described herein. In further embodiments, the respiratory-condition score may indicate a likelihood that a user will develop, will still have, or will recover from a respiratory condition within a future time interval. The example scenario of FIG. 4E depicts an embodiment in which it is predicted that a user, who is suffering from cold, will feel better within the next three days.

In some embodiments, contextual information may be utilized, in addition to the user's voice information, to determine or predict a user's respiratory condition. As further described herein, the contextual information may include, without limitation, physiological data for the user, such as body temperature, sleep data, mobility information, self-reported symptoms, location, or weather-related information. Self-reported symptom data may include, for example, whether the user is feeling a particular symptom or not, such as congestion, and may further include a degree or rating of severity for experiencing the symptom. In some instances, a symptom self-reporting tool may be utilized to acquire user symptom information. In some embodiments, automatic prompting to provide self-reported information (or a notification requesting the user to report symptom data) may occur based on an analysis of the user's voice-related data or a determined respiratory condition for the user. The example scenario of FIG. 4D depicts an embodiment in which it is determined that the user may be getting sick based on analysis of the user's voice. In this embodiment, a monitoring software application may ask the user, for example, whether the user is feeling certain respiratory-related symptoms (e.g., congestion, tired, etc.). The example of FIG. 4D further depicts that, once the user affirms about the congestion, the user is prompted to rate the severity of the congestion. This user's self-reported symptoms may be utilized to make additional determinations or forecasts about the user's respiratory condition. In some embodiments, other contextual information may be utilized, such as physiological data (such as heart rate, body temperature, sleep, or other data) of the user, weather-related information (e.g., humidity, temperature, pollution or similar data), location or other contextual information described herein, such as information about respiratory-infection outbreaks in the user's region.

Based on a determination of the user's respiratory condition, which may include a change (or lack of change) in the condition, a computing device may initiate an action. The action may comprise, for example, electronically communicating an alert or a notification to the user, a clinician, or a caregiver for the user. In some embodiments, the notification or alert may include information about the user's respiratory condition such as a respiratory-condition score, information quantifying or characterizing a change in the user's respiratory condition, a current state of the respiratory condition, and/or a prediction of the user's respiratory condition in the future. In some embodiments, an action may further include processing the respiratory condition information for decision-making, which may include providing a recommendation for treatment and support based on a user's respiratory condition. For example, the recommendation might comprise consulting with a healthcare provider, continuing an existing prescription or over-the-counter medicine (such as re-fill a prescription), modifying a dosage or medication of a current treatment protocol, and/or modifying or not modifying (i.e., continuing) the monitoring of the respiratory condition. In some aspects, the action may include initiating one or more of these or other recommendations, such as automatically scheduling an appointment with the user's healthcare provider and/or communicating a notification to a pharmacy for re-filling a prescription. The example scenario of FIG. 4F depicts an embodiment in which, based on a determination that the user's respiratory condition is not improving, a user's doctor is notified and a prescription for antibiotics is refilled and scheduled for delivery to the user.

Still another type of action may comprise automatically initiating or performing an operation associated with the monitoring or treatment of the user's respiratory condition. By way of example, and without limitation, this operation may include automatically scheduling an appointment with the user's healthcare provider, sending a notification to a pharmacy for re-filling a prescription, or modifying procedures associated with, or the computer operations utilized for, monitoring user's respiratory condition. In one embodiment of an example action, voice analysis procedures, such as computer programming operations utilized for obtaining or analyzing user voice-related data, are modified. In one such embodiment, a user may be prompted to provide voice samples more frequently, such as twice per day, or voice information may be collected more frequently, such as in the embodiments where voice information is collected from casual interactions with a computing device. In another such embodiment, the particular phoneme(s) or feature information, collected or analyzed by a respiratory-condition monitoring application, may be modified. In one embodiment, computer programming operations may be modified such that the user may be instructed to make a different set of sounds than the sounds they have been provided previously. Similarly, in another type of action, computer programming operations may be modified to prompt the user to provide symptom data, such as described previously.

Among others, one benefit that may be provided by embodiments of the technologies disclosed herein is the early detection of a respiratory condition, such as an infection. In accordance with these embodiments, acoustic features of user vocalizations, including respiratory sounds, may be utilized to detect even mild respiratory symptoms or manifestations of a respiratory condition and alert an individual or a healthcare provider of a condition before the individual suspects an illness (e.g., before the user feels symptomatic). Early detection of respiratory conditions may lead to a more effective intervention that reduces the duration and/or severity of the infection. Early detection of respiratory infections may also reduce the risk of transmission to other individuals, as it enables the infected individual to take precautions against transmission, such as wearing a mask or self-quarantining, sooner than they otherwise would follow. In this way, these embodiments provide an improvement over conventional approaches for respiratory condition, including respiratory infection, detection, which depend on the user reporting symptoms and, thus, make a condition being detected later (or not at all). These conventional approaches also are less accurate or imprecise due to subjectivity of the user's self-reported data.

Early detection of respiratory infections may also be beneficial in clinical trials. For example, in a clinical trial for a vaccine, a confirmation of a correlation between an individual's symptoms and the infection of interest is required. If the individual is not diagnosed early enough, the infectious agent load in the individual drops too low that it may not be possible to confirm the correlation of the individual's symptoms to the infection of interest. Without confirmation, the individual could not participate in the trial. Accordingly, the embodiments described herein may be utilized in not only making early detection for more effective treatments, but also when utilized for clinical trials, these embodiments may enable higher trial participation for developing new potential treatments or vaccines.

Another benefit that may be provided by embodiments of the technologies disclosed herein is an increased likelihood of user compliance for monitoring respiratory conditions. For instance, and as further described herein, user's voice recordings may be obtained unobtrusively, at home or away from a doctor's clinic, and, in some aspects, during the time when the individual is performing daily routines, for example, carrying out everyday conversations, where there is little burden on the individual. A less burdensome manner for monitoring respiratory conditions, including obtaining user data, may increase user compliance, which in turn may help to ensure early detection and may provide another improvement over conventional approaches to monitor respiratory condition.

Still another benefit that may be provided by embodiments of the technologies disclosed herein is improved accuracy in treating individuals with respiratory conditions. In particular, some of the embodiments of this disclosure enable tracking a potential respiratory condition, such as an infection, to determine whether the condition is worsening, improving, or not changing, which may impact the individual's treatment. For example, an individual with initially mild symptoms may not need to medicate or receive treatment right away. Some embodiments of this disclosure may be utilized to monitor the progress of the condition and alert the individual and/or a healthcare provider if the condition worsens to the point that treatment (e.g., medication) may be needed or is recommended. Additionally, embodiments of this disclosure may determine whether an individual is recovering from a respiratory condition such as an infection or not and, therefore, whether a change in treatment, such as changing medication and/or dosage, is recommended or not. In another example, embodiments of this disclosure may determine a user's respiratory condition when the user is prescribed a medication with potential respiratory-related side effects, such as certain cancer-treating medications, and determine whether a change in treatment is recommended based on whether and to what extent the user is experiencing the respiratory-related side effects. In this way, some embodiments of the technologies described herein may provide improvement on the conventional technologies by enabling more precise utilization of medicines, and in particular, medicines such as antibiotics/anti-microbial medicines, as such medicines may be prescribed or continued based on objective, quantifiable detected change(s) in an individual's respiratory condition.

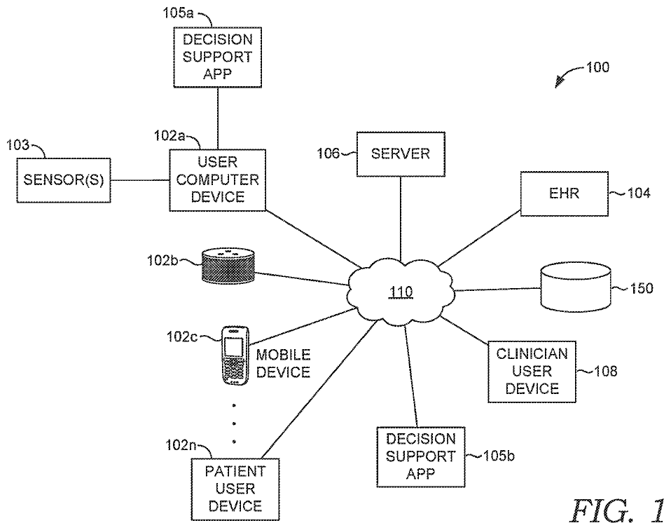

Turning now to FIG. 1, a block diagram is provided showing an example operating environment 100 in which some embodiments of the present disclosure may be employed. It should be understood that this and other arrangements described herein are set forth only as examples. Other arrangements and elements (e.g., machines, interfaces, functions, orders, and groupings of functions) may be used in addition to, or instead of, those shown in FIG. 1 as well as other figures, and some elements may be omitted altogether for the sake of clarity. Further, many of the elements described herein are functional entities that may be implemented as discrete or distributed components, or in conjunction with other components, and in any suitable combination and location. Various functions or operations described herein are being performed by one or more entities including a hardware, firmware, software, and a combination thereof. For instance, some functions may be carried out by a processor executing instructions stored in a memory.

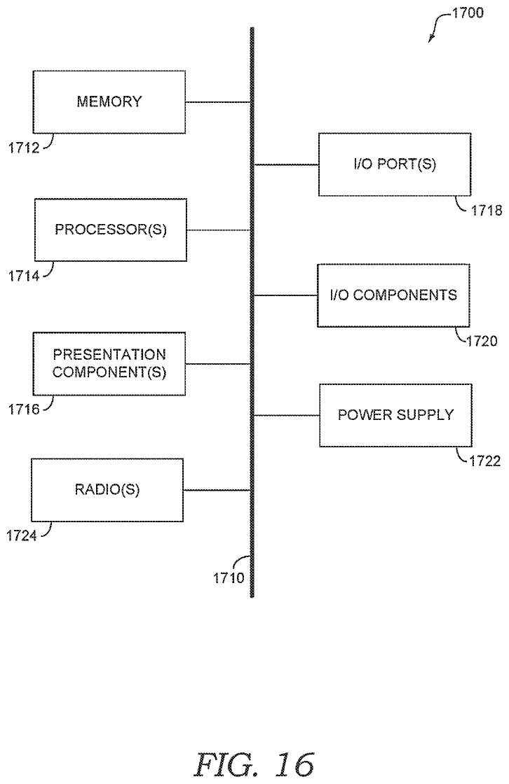

As shown in FIG. 1, example operating environment 100 includes a number of user devices, such as user computer devices (interchangeably referred as “user devices”) 102a, 102b, 102c through 102n and a clinician user device 108; one or more decision support applications, such as decision support applications 105a and 105b; an electronic health record (EHR) 104; one or more data sources, such as a data store 150; a server 106; one or more sensors, such as a sensor(s) 103; and a network 110. It should be understood that operating environment 100 shown in FIG. 1 is an example of one suitable operating environment. Each of the components shown in FIG. 1 may be implemented via any type of computing device, such as a computing device 1700 described in connection with FIG. 16, for example. These components may communicate with each other via network 110, which may include, without limitation, one or more local area networks (LANs) and/or wide area networks (WANs). In exemplary implementations, network 110 may comprise Internet and/or a cellular network, amongst any of a variety of possible public and/or private networks.

It should be understood that any number of user devices (such as 102a-n and 108), servers (such as 106), decision support applications (such as 105a-b), data sources (such as data store 150), and EHRs (such as 104) may be employed within operating environment 100 within the scope of the present disclosure. Each element may comprise a single device or a component, or multiple devices or components, cooperating in a distributed environment. For instance, server 106 may be provided via multiple devices arranged in a distributed environment that collectively provide the functionality described herein. Additionally, other components not shown herein may also be included within the distributed environment.

User devices 102a, 102b, 102c through 102n and clinician user device 108 may be client user devices on a client-side of operating environment 100, while server 106 may be on a server-side of operating environment 100. Server 106 may comprise server-side software designed to work in conjunction with client-side software on user devices 102a, 102b, 102c through 102n and 108 to implement any combination of the features and functionalities discussed in the present disclosure. This division of operating environment 100 is provided to illustrate one example of a suitable environment, and there is no requirement that any combination of server 106 and user devices 102a, 102b, 102c through 102n and 108 remain as separate entities.

User devices 102a, 102b, 102c through 102n and 108 may comprise any type of computing device capable of use by a user. For example, in one embodiment, user devices 102a, 102b, 102c through 102n and 108 may be the type of computing devices described in relation to FIG. 16 herein. By way of example, and not limitation, a user device may be embodied as a personal computer (PC), a laptop computer, a mobile or a mobile device, a smartphone, a smart speaker, a tablet computer, a smartwatch, a wearable computer, a personal digital assistant (PDA) device, a music player or an MP3 player, a global positioning system (GPS), a video player, a handheld communications device, a gaming device, an entertainment system, a vehicle computer system, an embedded system controller, a camera, a remote control, an appliance, a consumer electronic device, a workstation, or any combination of these delineated devices, or any other suitable computer device.

Some user devices, such as user devices 102a, 102b, 102c through 102n may be intended to be used by a user who is being observed via one or more sensors, such as sensor(s) 103. In some embodiments, a user device may include an integrated sensor (similar to sensor(s) 103) or operate in conjunction with external sensor (similar to 103). In exemplary embodiments, sensor(s) 103 senses acoustic information. For example, sensor(s) 103 may comprise one or more microphones (or microphone arrays) implemented with, or through, communicatively coupled to a smart device, such as a smart speaker, a smart mobile device, a smartwatch or as a separate microphone device. Other types of sensors may also be integrated into or work in conjunction with user devices, such as physiological sensors (e.g., sensors detecting heart rate, blood pressure, blood oxygen levels, temperature and related data). However, it is contemplated, that physiological information about an individual, according to embodiments of the disclosure, may also be received from the individual's historical data in EHR 104, or from human measurements or human observations. Additional types of sensors that may be implemented in operating environment 100 include sensors configured to detect user location (e.g., an indoor positioning system (IPS) or a global positioning system (GPS)); atmospheric information (e.g., a thermometer, a hygrometer or a barometer); ambient light (e.g., a photodetector); and motion (e.g., a gyroscope or an accelerometer).

In some aspects, sensor(s) 103 may be operable with or through a smartphone carried by the user (such as user device 102c) or a smart speaker positioned in one or more areas in which the individual may be located (such as user device 102b). For example, sensor(s) 103 may be a microphone integrated into a smart speaker located in an individual's home that may sense sound information, including the user's voice, occurring within a maximum distance from the smart speaker. It is contemplated that sensor(s) 103 may alternatively be integrated in other manners, such as sensors integrated into a device positioned on or near a wearer's body. In other aspects, sensor(s) 103 may be a skin-patch sensor adhered to the user's skin; an ingestible or sub-dermal sensor, or sensor components integrated into the user's living environment (including a television, a thermostat, a doorbell, a camera or other appliances).

Data may be acquired by sensor(s) 103 continuously, periodically, as needed, or as it becomes available. Further, data acquired by sensor(s) 103 may be associated with time and date information and may be represented as one or more time series of measured variables. In an embodiment, sensor(s) 103 may collect raw sensor information and may perform signal processing, form variable decision statistics, cumulative summing, trending, wavelet processing, thresholding, computational processing of decision statistics, logical processing of decision statistics, pre-processing and/or signal condition. In some embodiments, sensor(s) 103 may comprise an analog-to-digital converter (ADC) and/or processing functionality for performing digital audio sampling of analog audio information. In some embodiments, the analog-to-digital converter and/or processing functionality for performing digital audio sampling to determine digital audio information may be implemented on any of the user devices 102a-n or on server 106. Alternatively, one or more of these signal processing functions may be performed by a user device, such as user devices 102a-n or clinician user device 108, server 106, and/or decision support applications (apps) 105a or 105b.

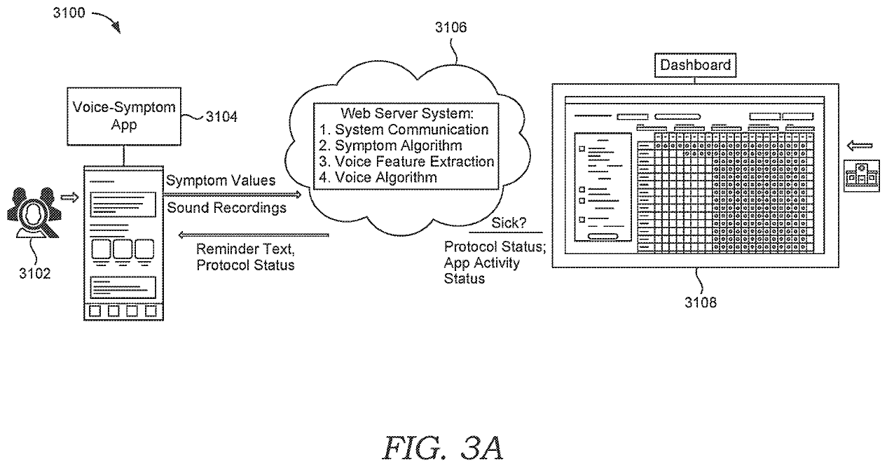

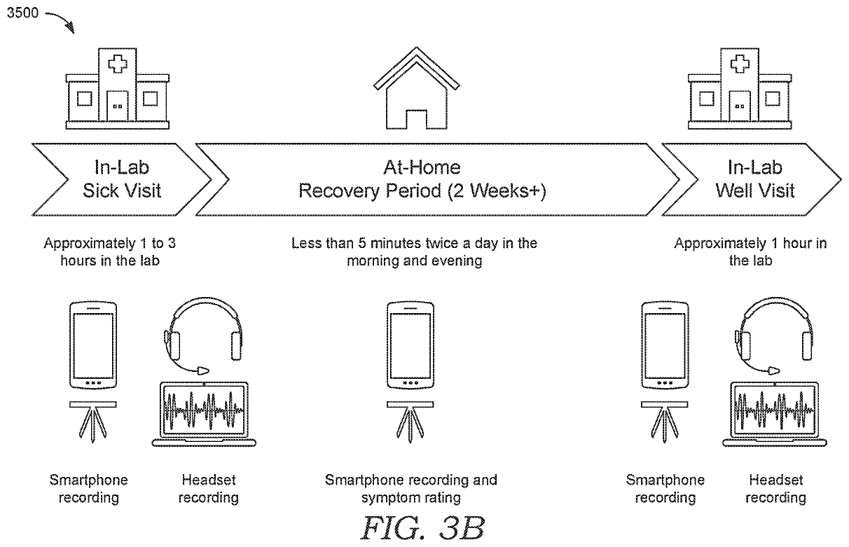

Some user devices, such as clinician user device 108, may be configured for use by a clinician who is treating or otherwise monitoring a user associated with sensor(s) 103. Clinician user device 108 may be embodied as one or more computing devices, such as user devices 102a-n or server 106 and is communicatively coupled through network 110 to EHR 104. Operating environment 100 depicts an indirect communicative coupling between clinician user device 108 and EHR 104 through network 110. However, it is contemplated that an embodiment of clinician user device 108 may be communicatively coupled to EHR 104 directly. An embodiment of clinician user device 108 may include a user interface (not shown in FIG. 1), operated by a software application or a set of applications, on clinician user device 108. In one embodiment, the application may be a Web-based application or applet. One example of this application comprises a clinician dashboard, such as an example dashboard 3108 described in connection with FIG. 3A. In accordance with embodiments described herein, a healthcare provider application (e.g., a clinician application such as a dashboard application, which may operate on clinician user device 108) may facilitate accessing and receiving information about a specific patient or a set of patients for which acoustic features and/or respiratory condition data may be determined. Some embodiments of clinician user device 108 (or a clinician application operating thereon) may further facilitate accessing and receiving information about a specific patient or a set of patients including patient history; healthcare resource data; physiological variables or data (e.g., vital signs); measurements; time series; predictions (including plotting or displaying a determined outcome and/or issuing an alert) described later; or other health-related information. The clinician user device 108 may further facilitate display of results, recommendations, or orders, for example. In an embodiment, clinician user device 108 may facilitate receiving orders for a patient based on the results of monitoring of respiratory-condition and determinations or predictions described herein. Clinician user device 108 may also be used to provide diagnostic services or evaluation of the performance of the technology described herein in conjunction with various embodiments.

Embodiments of decision support applications 105a and 105b may comprise a software application or a set of applications (which may include programs, routines, functions, or computer-performed services) residing on one or more servers, distributed in a cloud-computing environment (e.g., decision support application 105b), or residing on one or more client computing devices (e.g., decision support application 105a) such as a personal computer, a laptop, a smartphone, a tablet, a mobile computing device, or front-end terminal in communication with back-end computing systems, or any of user devices 102a-n. In an embodiment, decision support applications 105a and 105b may include a client-based and/or Web-based application (or app), or a set of applications (or apps), usable to access user services provided by an embodiment of this disclosure. In one such embodiment, each of the decision support applications 105a and 105b may facilitate processing, interpreting, accessing, storing, retrieving, and communicating information acquired from user devices 102a-n, clinician user device 108, sensor(s) 103, EHR 104, or data store 150, including predictions and evaluations determined by embodiments of this disclosure.

Utilization and retrieval of information through decision support applications 105a and 105b or utilization of associated functionality may require a user, such as a patient or a clinician, to login with credentials. Further, decision support applications 105a and 105b may store and transmit data in accordance with privacy settings defined by clinician, patient, an associated healthcare facility or system, and/or applicable local and federal rules and regulations regarding protecting health information, such as Health Insurance Portability and Accountability Act (HIPAA) rules and regulations.

In an embodiment, decision support applications 105a and 105b may communicate a notification (such as an alarm or an indication) directly to clinician user device 108 or user devices 102a-n through network 110. If these applications are not operating on these devices, they may surface the notification on any other device on which decision support applications 105a and 105b are operating. Decision support applications 105a and 105b may also send or surface maintenance indications to clinician user device 108 or user devices 102a-n. Further, an interface component may be used in decision support applications 105a and 105b to facilitate access by a user (including a clinician/caregiver or a patient) to functions or information on sensor(s) 103, such as operational settings or parameters, user identification, user data stored on sensor(s) 103, and diagnostic services or firmware updates for sensor(s) 103, for example.

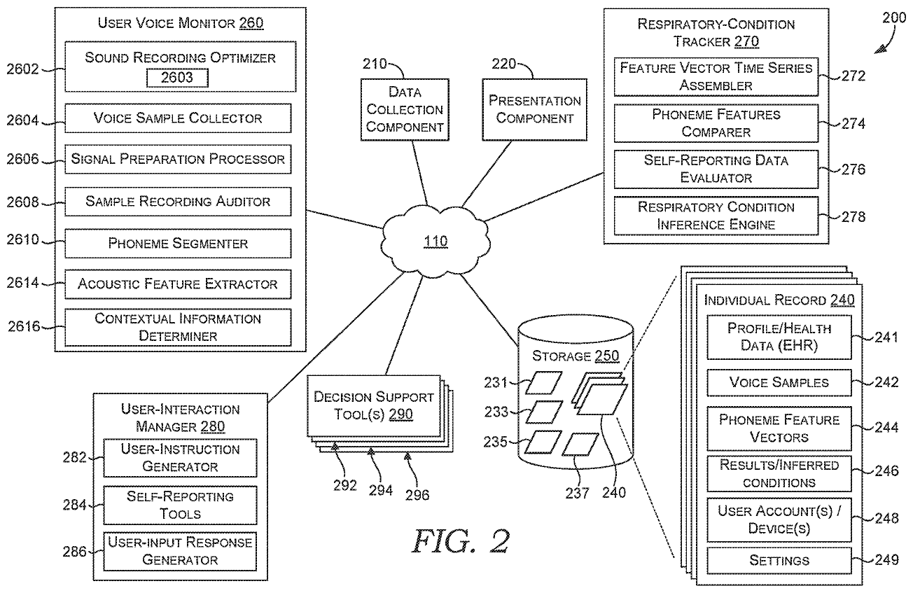

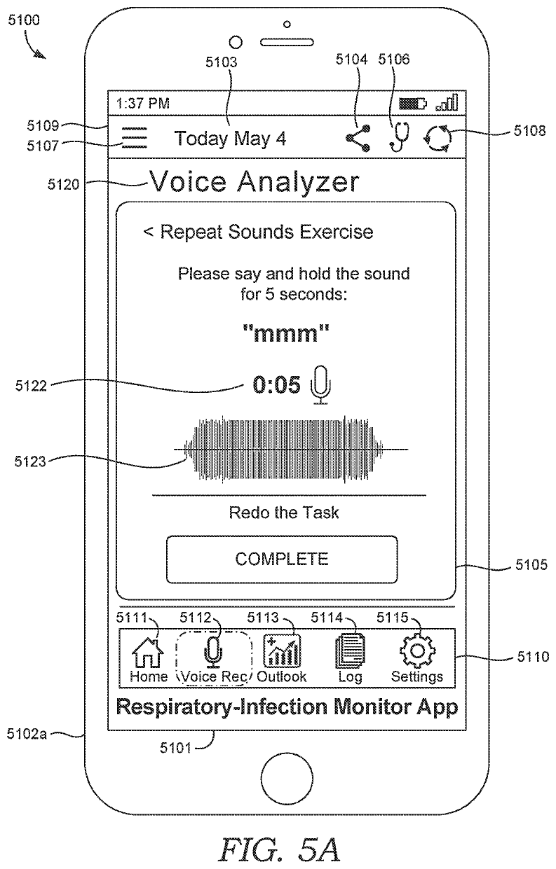

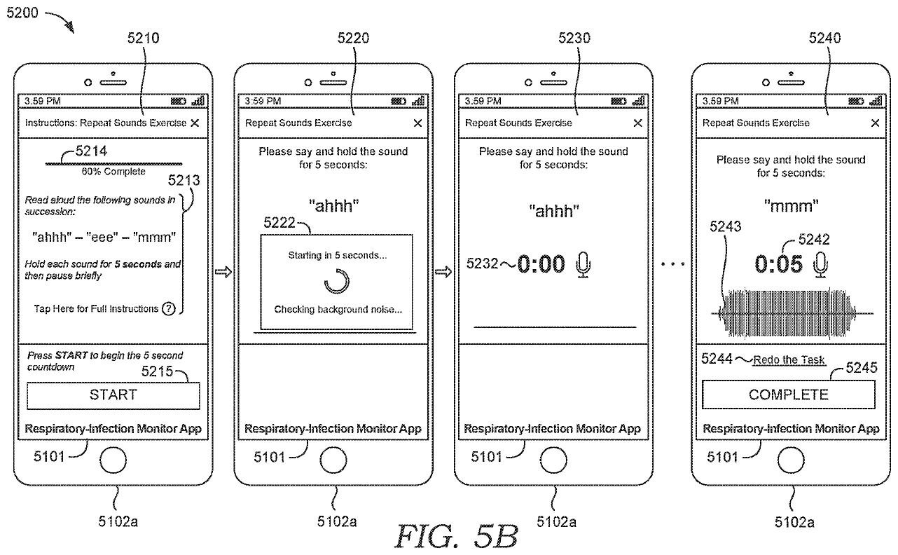

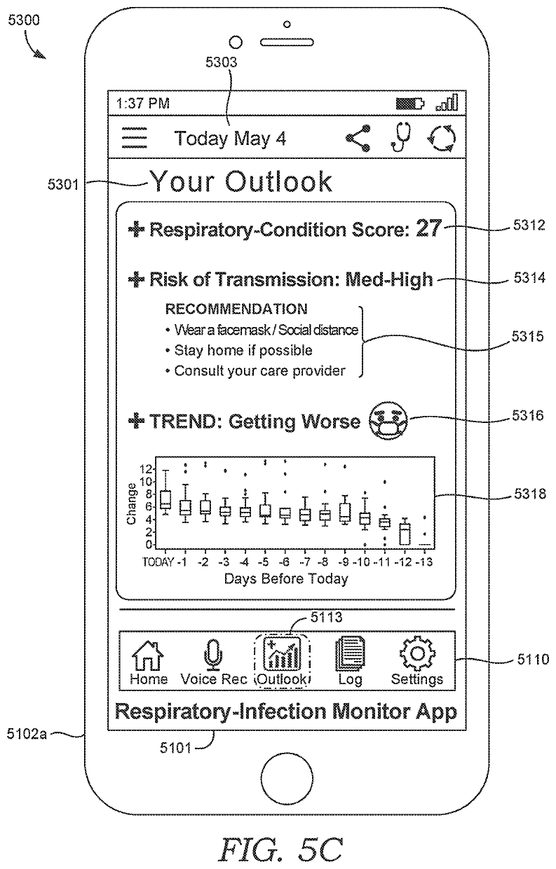

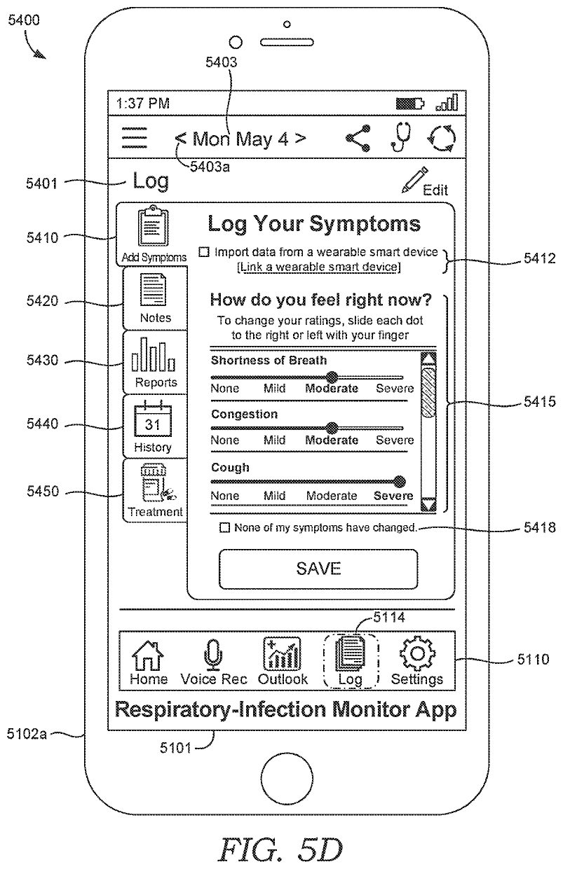

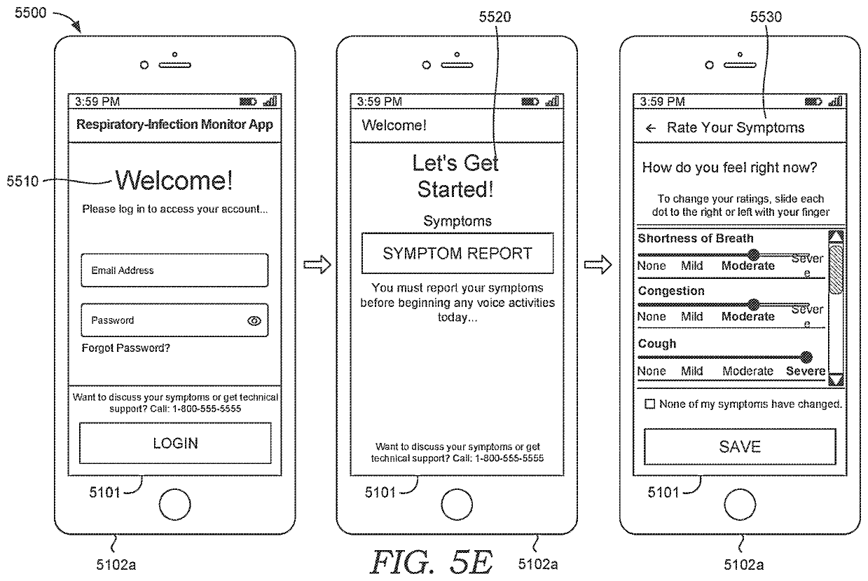

Further, embodiments of decision support applications 105a and 105b may collect sensor data directly or indirectly from sensor(s) 103. As described with respect to FIG. 2, decision support applications 105a and 105b may utilize the sensor data to extract or determine acoustic features and determine respiratory conditions and/or symptoms. In one aspect, decision support applications 105a and 105b may display or otherwise provide results of such processes to a user via a user device, such as user devices 102a-n and 108, including through various graphical, audio, or other user interfaces, such as the example graphic user interfaces (GUIs) depicted in FIGS. 5A-5E. In this way, the functionality of one or more components discussed below with respect to FIG. 2 may be performed by computer programs, routines, or services that operate in conjunction with or are part of or controlled by decision support applications 105a or 105b. In addition, or alternatively, decision support applications 105a and 105b may include decision support tools, such as a decision support tool(s) 290 of FIG. 2.

As mentioned above, operating environment 100 includes one or more EHRs 104, which may be associated with a monitored individual. EHR 104 may be directly or indirectly communicatively coupled to user devices 102a-n and 108, via network 110. In some embodiments, EHR 104 may represent health information from different sources and may be embodied as distinct records systems, such as separate EHR systems for different clinician user devices (such as 108). As a result, the clinician user devices (such as 108) may be for clinicians of different provider networks or care facilities.

Embodiments of EHR 104 may include one or more data stores of health records or health information, which may be stored on data store 150, and may further include one or more computers or servers (such as server 106) that facilitate storing and retrieving health records. In some embodiments, EHR 104 may be implemented as a cloud-based platform or may be distributed across multiple physical locations. EHR 104 may further include record systems that may store real-time or near real-time patient (or user) information, such as wearable, bedside, or in-home patient monitors, for example.

Data store 150 may represent one or more data sources and/or computer data storage systems, which are configured to make data available to any of the various components of operating environment 100 or a system 200, which is described in conjunction with FIG. 2. In one embodiment, data store 150 may provide (or make available for accessing) sensor data, which may be available to a data collection component 210 of system 200. Data store 150 may comprise a single data store or a plurality of data stores and may be locally and/or remotely located. Some embodiments of data store 150 may comprise networked storage or distributed storage including storage on servers (such as server 106) located in the cloud environment. Data store 150 may be discrete from user devices 102a-n and 108 and server 106 or may be incorporated and/or integrated with at least one of those devices.

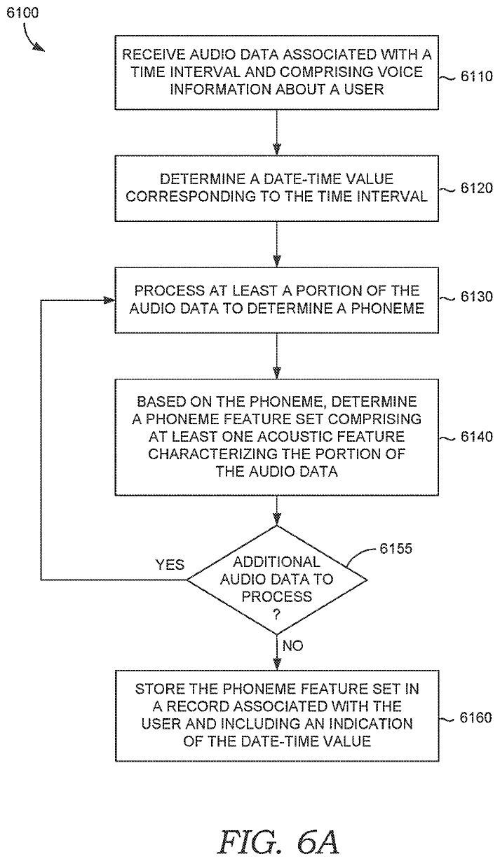

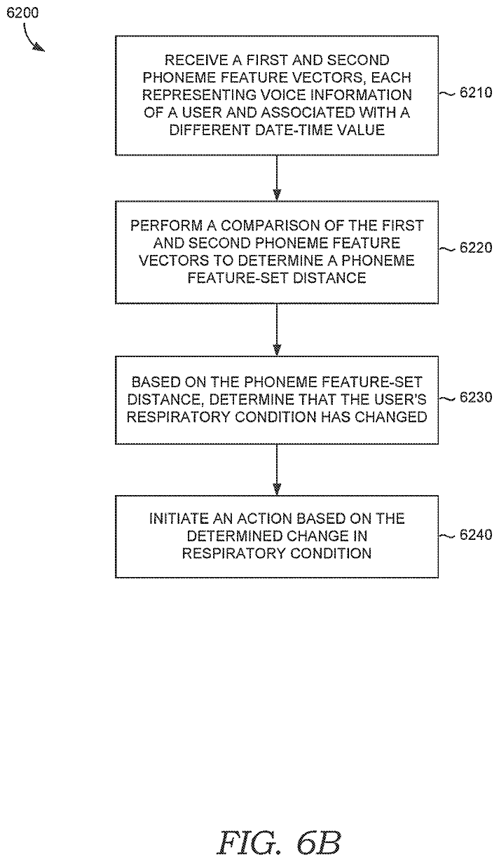

Operating environment 100 may be utilized to implement one or more components of system 200 (shown in and described in conjunction with FIG. 2) or the operations performed by these components, including components or operations for collecting voice data or contextual information; facilitating interactions with a user to collect such data; tracking a possible or known respiratory condition (e.g., a respiratory infection or non-infectious respiratory symptoms); and/or implementing a decision support tool (such as decision support tool(s) 290 of FIG. 2). Operating environment 100 may also be utilized for implementing aspects of methods 6100 and 6200, as described in conjunction with FIGS. 6A and 6B, respectively.

Referring now to FIG. 2 and with continuing reference to FIG. 1, a block diagram is provided showing aspects of an example computing system architecture suitable for implementing an embodiment of the present disclosure and designated generally as system 200. System 200 represents only one example of a suitable computing system architecture. Other arrangements and elements may be used in addition to, or instead of, those shown, and some elements may be omitted altogether for the sake of clarity. Further, similar to operating environment 100 of FIG. 1, many elements described herein are functional entities that may be implemented as discrete or distributed components or in conjunction with other components, and in any suitable combination and location.

Example system 200 includes network 110, which is described in connection with FIG. 1, and which communicatively couples components of system 200 including a data collection component 210, a presentation component 220, a user voice monitor 260, a user-interaction manager 280, a respiratory-condition tracker 270, a decision support tool(s) 290, and a storage 250. One or more of these components may be embodied as a set of compiled computer instructions or functions, program modules, computer software services, or an arrangement of processes carried out on one or more computer systems, such as computing device 1700 described in connection with FIG. 16, for example.

In one embodiment, the functions performed by components of system 200 are associated with one or more decision support applications, services, or routines (such as decision support applications 105a-b of FIG. 1). In particular, such applications, services, or routines may operate on one or more user devices (such as user device 102a and/or clinician user device 108) or servers (such as server 106), distributed across one or more user devices and servers, or implemented in the cloud environment (not shown). Moreover, in some embodiments, these components of system 200 may be distributed across a network, connecting one or more servers (such as server 106) and client devices (such as user computer devices 102a-n or clinician user device 108), in the cloud environment, or may reside on a user device, such as any of user devices 102a-n or clinician user device 108. Moreover, functions or services performed by these components may be implemented at appropriate abstraction layer(s) such as an operating system layer, an application layer, a hardware layer, or so on of the computing system(s). Alternatively, or in addition, the functionality of these components and/or the embodiments described herein may be performed, at least in part, by one or more hardware logic components. For example, and without limitation, illustrative types of hardware logic components that may be used include Field-Programmable Gate Arrays (FPGAs), Application-Specific Integrated Circuits (ASICs), Application-Specific Standard Products (ASSPs), System-on-a-Chip systems (SoCs), Complex Programmable Logic Devices (CPLDs), etc. Additionally, although functionality is described herein with regards to specific components shown in example system 200, it is contemplated that in some embodiments functionality of these components may be shared or distributed across other components.

Continuing with FIG. 2, data collection component 210 may generally be responsible for accessing or receiving (and in some cases identifying) data from one or more data sources, such as data from sensor(s) 103 and/or data store 150 of FIG. 1, to utilize in embodiments of the present disclosure. In some embodiments, data collection component 210 may be employed to facilitate accumulation of sensor data acquired for a particular user (or in some cases, a plurality of users including crowdsourced data) for other components of system 200, such as user voice monitor 260, user-interaction manager 280, and/or respiratory-condition tracker 270. This data may be received (or accessed), accumulated, reformatted, and/or combined by data collection component 210 and stored in one or more data stores such as storage 250, where it may be available to other components of system 200. For example, the user data may be stored in or associated with an individual record 240, as described herein. Additionally, or alternatively, in some embodiments, any personally identifiable data (i.e., user data that specifically identifies particular users) is not uploaded, otherwise provided from one or more data sources, not permanently stored, and/or not made available to other components of system 200. In one embodiment, user-related data is encrypted, or other security measures implemented so that user privacy is preserved. In another embodiment, a user may opt into or out of services provided by the technologies described herein and/or select which user data and/or which sources of user data are to be utilized by these technologies.

Data utilized in embodiments of the present disclosure may be received from a variety of sources and may be available in a variety of formats. For example, in some embodiments, user data received via data collection component 210 may be determined via one or more sensors (such as sensor(s) 103 of FIG. 1), which may be stored on or associated with one or more user devices (such as user device 102a), servers (such as server 106), and/or other computing devices. As used herein, a sensor may include a function, a routine, a component, or a combination thereof for sensing, detecting, or otherwise obtaining information, such as user data from data store 150, and may be embodied as hardware, software, or both. As mentioned earlier, by way of example and not limitation, data that is sensed or determined from one or more sensors may include acoustic information (including information from user speech, utterances, breathing, coughing, or other vocal sounds); location information, such as an Indoor Positioning System (IPS) or Global Positioning System (GPS) data, which may be determined from a mobile device; atmospheric information, such as temperature, humidity, and/or pollution; physiological information, such as body temperature, heart rate, blood pressure, blood oxygen levels, sleep-related information; motion information, such as accelerometer or gyroscope data; and/or ambient light information, such as photodetector information.

In some aspects, sensor information collected by data collection component 210 may include further properties or characteristics of the user device(s) (such as a device state, charging data, date/time, or other information derived from a user device such as a mobile device or smart speaker); user-activity information (for example, app usage, online activity, online search, voice data such as automatic speech recognition, or activity log) including, in some embodiments, user activity that occurs on more than one user device; user history; session logs; application data; contacts; calendar and schedule data; notification data; social-network data; news (including e.g., popular or trending items on search engines, social networks, health department notifications, which may provide information about numbers or rates of respiratory-infections in a geographical region); ecommerce activity (including data from online accounts such as, Amazon.com®, Google®, eBay®, PayPal®, etc.); user-account(s) data (which may include data from user preferences or settings associated with a personal assistant application or service); home-sensor data; appliance data; vehicle signal data; traffic data; other wearable device data; other user device data (for example, device settings, profiles, network-related information (e.g., a network name or ID, domain information, workgroup information, connection data, wireless fidelity (Wi-Fi) network data, or configuration data, data regarding a model number, firmware, an equipment, device pairings, such as where a user has a mobile phone paired with a Bluetooth headset, or other network-related information)); payment or credit card usage data (may include information from a user's PayPal® account, for example); purchase history data (such as information from a user's Amazon.com® or online drugstore account); other sensor data that may be sensed or otherwise detected by a sensor (or other detector) component(s) including data derived from a sensor component associated with the user (including location, motion, orientation, position, user-access, user-activity, network-access, user-device-charging, or other data that is capable of being provided by one or more sensor components); data derived based on other data (for example, location data that may be derived from Wi-Fi, Cellular network, or Internet Protocol (IP) address data); and nearly any other source of data that may be sensed or determined, as described herein.

In some aspects, data collection component 210 may provide data collected in the form of data streams or signals. A “signal” may be a feed or stream of data from a corresponding data source. For example, a user signal could be user data acquired from a smart speaker, a smartphone, a wearable device (e.g., a fitness tracker or a smartwatch), a home-sensor device, a GPS device (e.g., for location coordinates), a vehicle-sensor device, a user device, a calendar service, an email account, a credit card account, a subscription service, a news or notifications feed, a website, a portal, or any other data sources. In some embodiments, data collection component 210 receives or accesses data continuously, periodically, or on as needed basis.

Further, user voice monitor 260 of operating environment 200 may generally be responsible for collecting or determining user voice-related data that may be utilized for detecting or monitoring respiratory condition. The term voice-related data (interchangeably referred herein as “voice data” or “voice information”) is used broadly herein and may comprise, by way of example and without limitation, data related to user speech, utterances including vocalizations or vocal sounds, or other sounds generated by the user's mouth or nose, such as breathing, coughing, sneezing, or sniffing. Embodiments of user voice monitor 260 may facilitate obtaining audio or acoustic information (e.g., audio recordings of vocalizations or voice samples), and in some aspects, contextual information, which may be received by data collection component 210. Embodiments of user voice monitor 260 may determine relevant voice-related information, such as phoneme features, from this audio data. User voice monitor 260 may receive data continuously, periodically, or on an as needed basis and, similarly, may extract or otherwise determine the voice information utilized for monitoring respiratory conditions on a continuous, periodic, or on an as needed basis.

In the example embodiment of system 200, user voice monitor 260 may comprise a sound recording optimizer 2602, a voice sample collector 2604, a signal preparation processor 2606, a sample recording auditor 2608, a phoneme segmenter 2610, an acoustic feature extractor 2614, and a contextual information determiner 2616. In another embodiment of user voice monitor 260 (not shown) only some of these subcomponents may be included or additional sub-components may be added. As explained further herein, one or more components of user voice monitor 260, such as signal preparation processor 2606, may perform pre-processing operations on audio data, such as raw acoustic data. It is contemplated that, in some embodiments, additional pre-processing may be done in accordance with data collection component 210.

Sound recording optimizer 2602 may be generally responsible for determining a proper or optimized configuration for obtaining useable audio data. As described above, it is contemplated that embodiments of the technology described herein may be utilized in an at-home environment or by an end-user in a setting other than a controlled environment, such as a lab or a doctor's clinic office. Accordingly, some embodiments may include functionality to facilitate obtaining audio data of sufficient quality to be used for monitoring a user's respiratory condition. In particular, in one embodiment, sound recording optimizer 2602 may be utilized to provide such functionality by providing an optimized configuration for obtaining audio data voice-related information. In one exemplary embodiment, an optimized configuration may be provided by tuning sensors or modifying other acoustic parameters (e.g., microphone parameters), such as signal strength, directivity, sensitivity, frequency, and signal to noise ratio (SNR). Sound recording optimizer 2602 may determine that the settings are within a pre-determined range for proper configuration or satisfy a pre-determined threshold (e.g., the microphone sensitivity or level is sufficiently adjusted to enable the user's voice data to be obtained from audio data). In some embodiments, sound recording optimizer 2602 may determine whether recording is initiated or not. In some embodiments, sound recording optimizer 2602 may also determine whether a sampling rate satisfies a threshold sampling rate or not. In one exemplary embodiment, sound recording optimizer 2602 may determine that the audio signal is sampled at a Nyquist rate, which in some instances comprises a minimum rate of 44.1 kilohertz (kHz). Additionally, sound recording optimizer 2602 may determine that a bit depth satisfies a threshold, such as 16 bits. Further, in some embodiments, sound recording optimizer 2602 may determine whether a microphone is tuned or not.

In some embodiments, sound recording optimizer 2602 may perform an initialization mode to optimize microphone levels for a particular environment in which the microphone is located. The initialization mode may include prompting a user to play a sound or make a noise in order for sound recording optimizer 2602 to determine the appropriate levels for the particular environment. In the initialization mode, sound recording optimizer 2602 may also prompt a user to stand or position themselves where the user normally stands or would position themselves in relation to the microphone when requesting user input. Based on user feedback (i.e., voice recordings), during initialization mode, sound recording optimizer 2602 may determine ranges, thresholds, and/or other parameters to configure the audio collection and processing components to provide an optimized configuration for future recording sessions. In some embodiments, sound recording optimizer 2602 may additionally or alternatively determine signal processing functions or configurations (e.g., noise cancellation, as described below) to facilitate obtaining usable audio data.

In some embodiments, sound recording optimizer 2602 may work in conjunction with signal preparation processor 2606 for pre-processing to make the optimized adjustments (e.g., adjust or amplify levels) to achieve a suitable configuration. Alternatively, sound recording optimizer 2602 may configure a sensor to achieve levels within a pre-determined range or threshold for a particular parameter, such as signal strength.

As shown in FIG. 2, sound recording optimizer 2602 may include a background noise analyzer 2603 that may generally be responsible for identifying and, in some embodiments, removing or reducing, background noise. In some embodiments, background noise analyzer 2603 may check that a noise intensity level satisfies a maximum threshold. For instance, background noise analyzer 2603 may determine that ambient noise in the user's recording environment is less than 30 decibel (dB). Background noise analyzer 2603 may check for speech (such as coming from a television or a radio). Background noise analyzer 2603 may also check for intermittent spikes or similar acoustic artifacts, which may be the result of a child yelling, a loud clock ticking, or a notification on a mobile device, for example.

In some embodiments, background noise analyzer 2603 may perform a background noise check, after recording has been initiated. In one such embodiment, the background noise check is done on a portion of the audio data received within a pre-determined time interval, prior to detection of a first phoneme in the recording (which may be detected, as described in conjunction with phoneme segmenter 2610). For example, background noise analyzer 2603 may perform a background noise check for five seconds prior to the start of the first phoneme in the audio data.

If background noise is detected, background noise analyzer 2603 may process (or attempt to process) the audio data to reduce or eliminate the noise. Alternatively, an indication of noise, determined by background noise analyzer 2603, may be provided to signal preparation processor 2606 to perform filtering and/or subtraction process to reduce or eliminate the noise. In some embodiments, in addition to or as an alternative to automatically reducing or eliminating background noise, background noise analyzer 2603 may send an indication informing the user (or other components of system 200, such as user-interaction manager 280) that the background noise is interfering or potentially interfering with voice collection and request the user to take an action to eliminate the background noise. For example, a notification may be provided to the user (e.g., via user interaction manager 280 or presentation component 220) to move to a quieter environment.

In some instances, after the audio data is obtained, background noise analyzer 2603 may re-check that audio data for the presence of background noise. For example, after sound recording optimizer 2602 (or in some embodiments, signal preparation processor 2606) automatically adjusts settings to reduce or eliminate noise, another check may be performed. In some aspects, subsequent checks may be performed as needed, at the beginning of a recording session, after a pre-determined period of time since the previous check, and/or if an indication is received, such as from the user, indicating that an action is taken to reduce or eliminate background noise.

Within user voice monitor 260, voice sample collector 2604 may generally be responsible for obtaining user's voice-related data in the form of an audio sample or a recording. Voice sample collector 2604 may operate in conjunction with data collection component 210 and user-interaction manager 280 to obtain samples of user's speech or other voice information. The audio sample may be in the form of one or more audio files that include recordings or samples of sustained phonemes, scripted speech, and/or unscripted speech. The term audio recording, as used herein, generally refers to a digital recording (e.g., an audio sample, which may be determined by audio sampling utilizing analog-to-digital conversion (ADC)).

In some embodiments, voice sample collector 2604 may include a functionality, such as ADC conversion functionality, for capturing and processing digital audio from analog audio (which may be received from sensor(s) 103 or an analog recording). In this way, some embodiments of voice sample collector 2604 may provide or facilitate determining a digital audio sample. In some embodiments, voice sample collector 2604 may also associate date-time information with the audio sample (e.g., timestamps an audio sample with a date and/or time) corresponding to a timeframe that the audio data is obtained. In one embodiment, the audio sample may be stored in an individual record associated with the user, such as voice samples 242 in individual record 240.

As described with respect to user-interaction manager 280 and depicted in the example of FIGS. 4A-4C and 5B, voice samples 242 may be obtained in response to the user participating in speech-related tasks. For example, and without limitation, a user may be asked to speak and hold a particular sound (e.g., “mmmm”) for a time interval or for as long as the user can, repeat certain words or phrases, read a passage, or be prompted to answer questions or engage in conversation so that voice samples 242 may be obtained. Voice samples 242 representing various types of speech-related tasks may be obtained from the user in the same collection session. For example, a user may be asked to speak and hold one or more phonemes for a certain time interval and speak and hold one or more phonemes for as long as the user can, where the latter phoneme(s) may be the same or different from the phoneme(s) held for a specified time interval. In some embodiments, a user may also be asked to read a written passage, which may have a variety of phonemes.

A voice sample herein refers to voice-related information in an audio sample, and may be determined from the audio sample, as described herein. For instance, the audio sample may include other acoustic information not related to the user's voice, such as background noise. Accordingly, in some instances, the voice sample may refer to a portion of an audio sample with voice-related information. In one embodiment, the voice sample may be determined from audio collected during a user's casual or day-to-day interaction with a user computing device (e.g., user device 102a of FIG. 1). For instance, a voice sample may be collected when a user states unprompted commands to a smart speaker or talks on a phone. In some embodiments, where voice sample information is obtained from the user's casual interaction with the user device, it may be unnecessary to prompt the user to participate in speech related tasks. Similarly, in some embodiments, the user may be prompted to complete speech related tasks for obtaining voice sample information that has not already been obtained via the user's speech from casual interaction, such as when information regarding a particular phoneme has not been obtained from the casual interaction speech.

As mentioned above, the technologies described herein provide for preserving and protecting user privacy. It is contemplated that embodiments that obtain audio samples from casual interaction with the user device may delete audio data once the voice-related data for respiratory-condition monitoring is determined. Similarly, the audio data may be encrypted and/or users may “opt in” to having voice-related data (for monitoring respiratory condition) collected from the so-called casual interactions.

Signal preparation processor 2606 may be generally responsible for preparing an audio sample for extracting voice-related information, such as phoneme features for further analysis. Accordingly, signal preparation processor 2606 may perform signal processing, pre-processing, and/or conditioning on audio data obtained or determined by voice sample collector 2604. In one embodiment, signal preparation processor 2606 may receive audio data from voice sample collector 2604 or may access voice sample data from voice samples 242 in individual record 240 associated with the user. Audio data that is prepared or processed by signal preparation processor 2606 may be stored as voice samples 242 and/or provided to other subcomponents of user voice monitor 260 or other components of system 200.

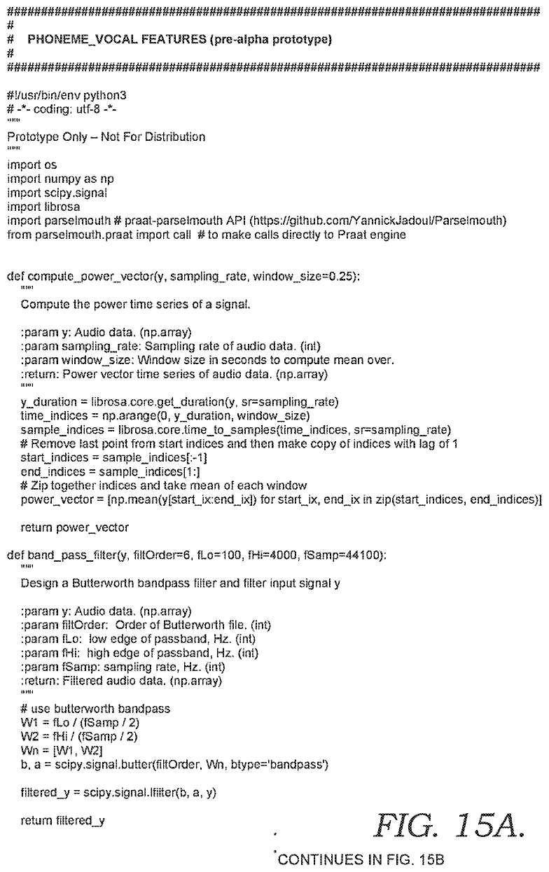

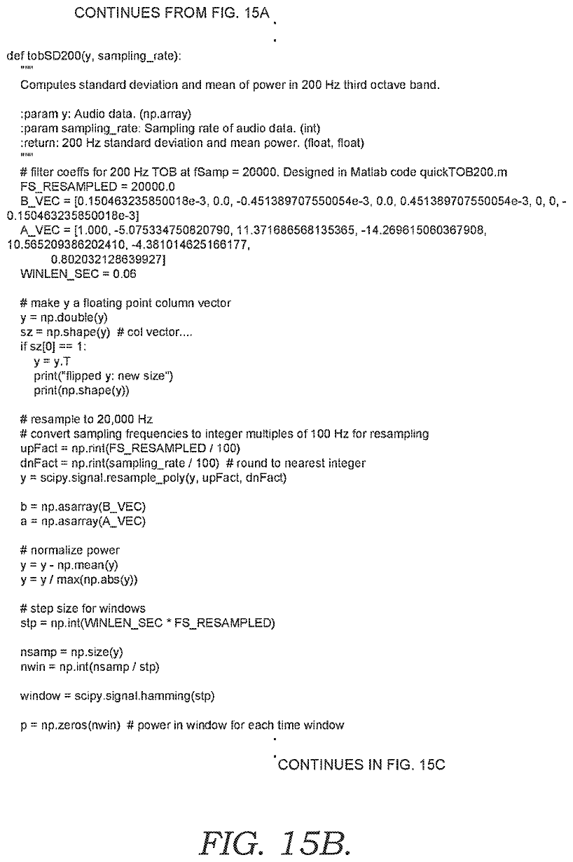

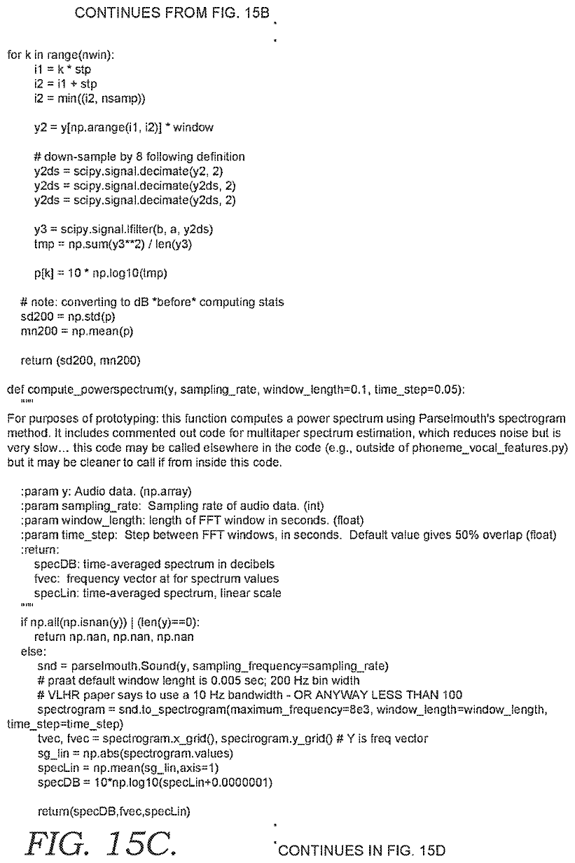

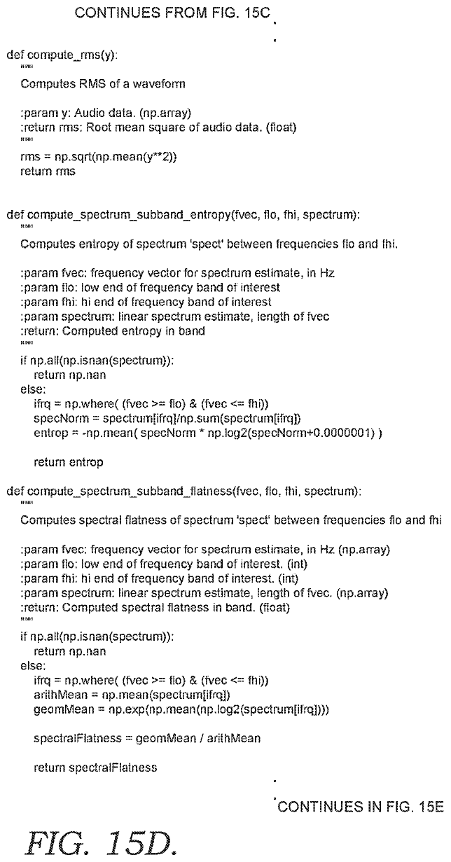

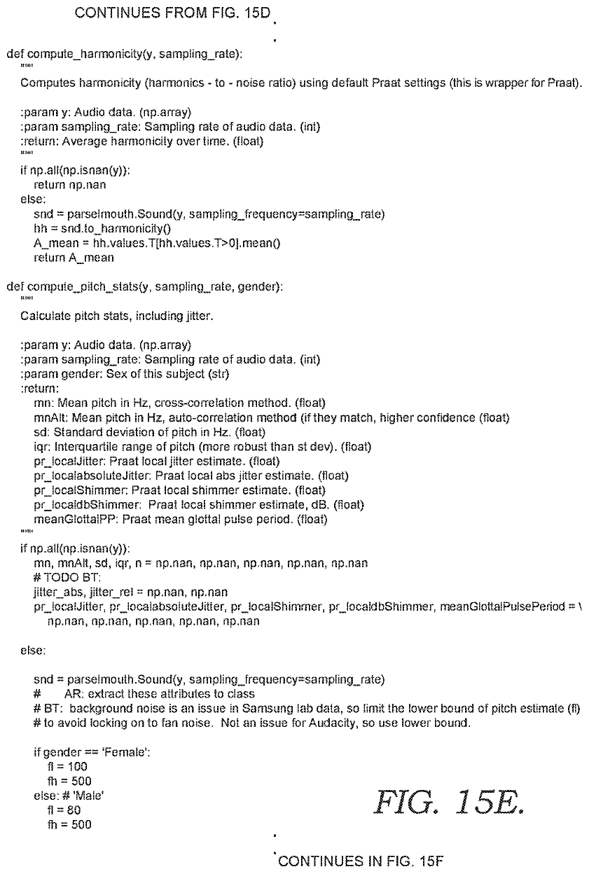

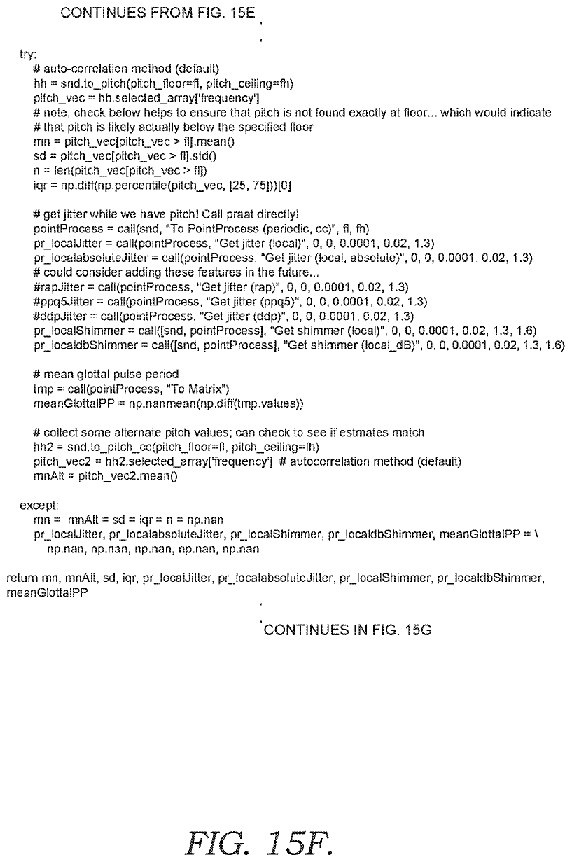

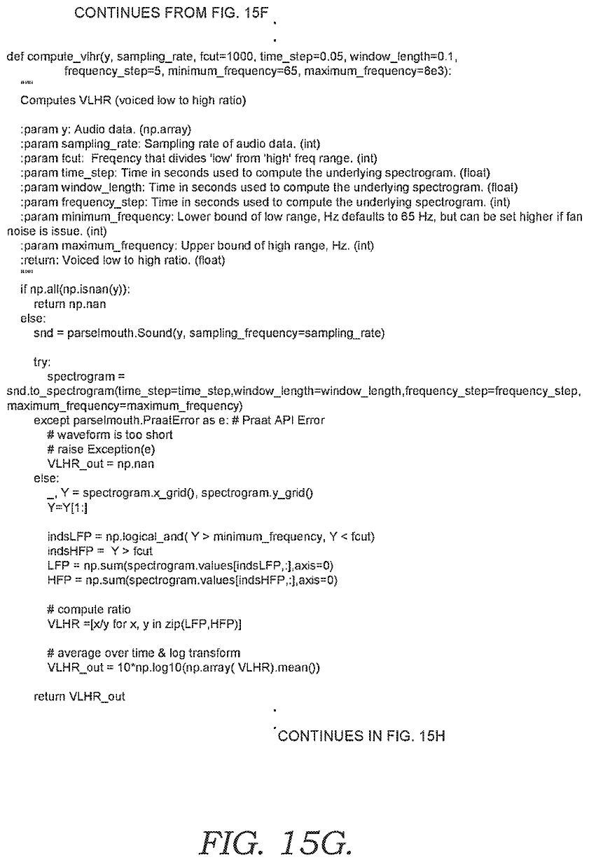

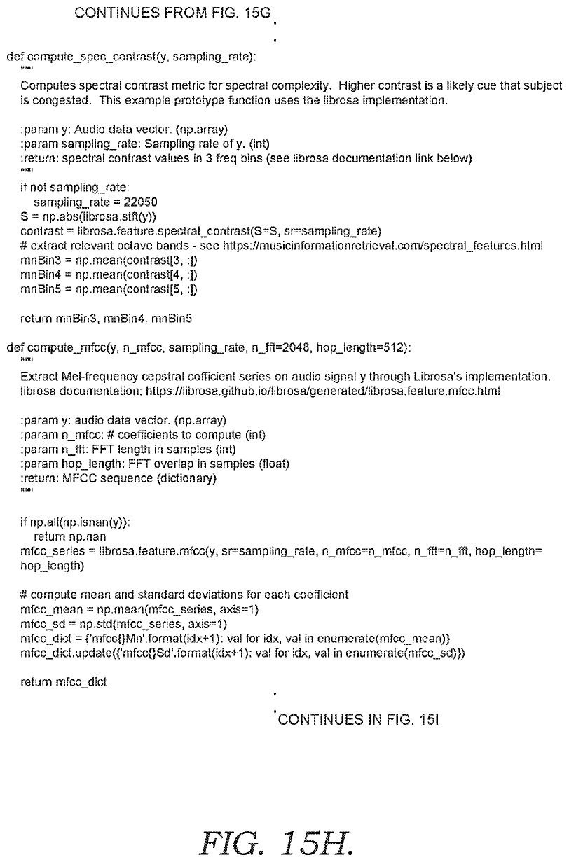

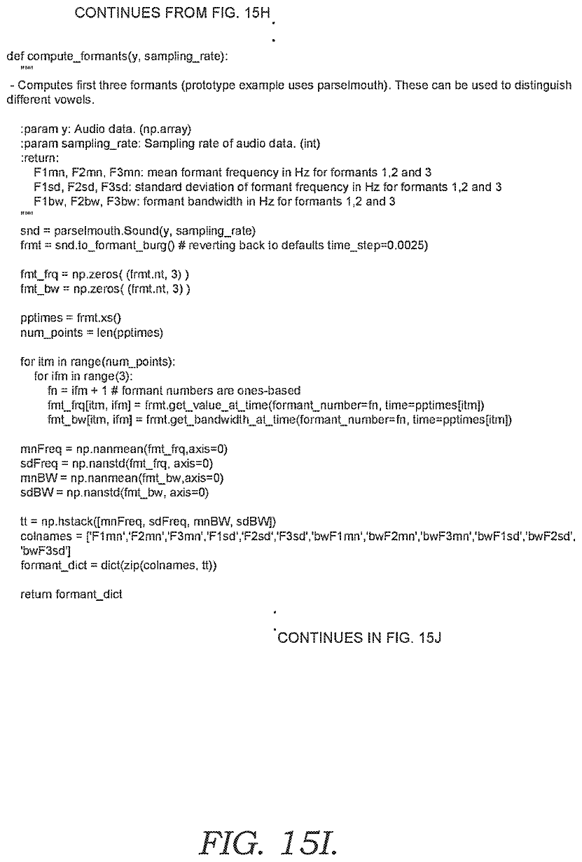

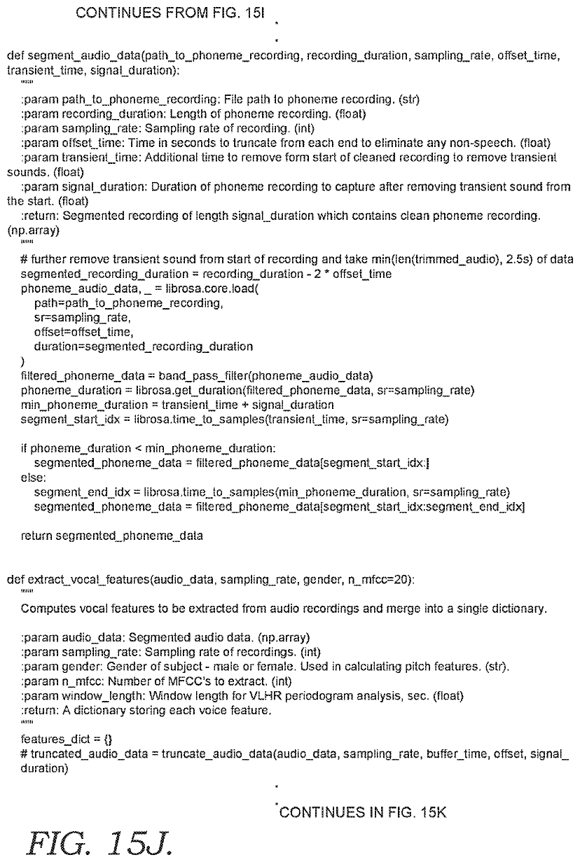

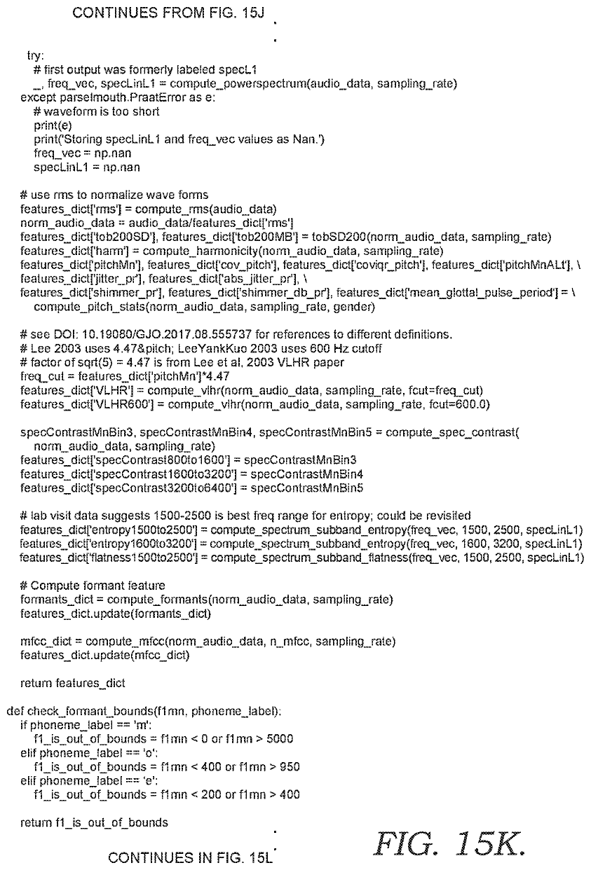

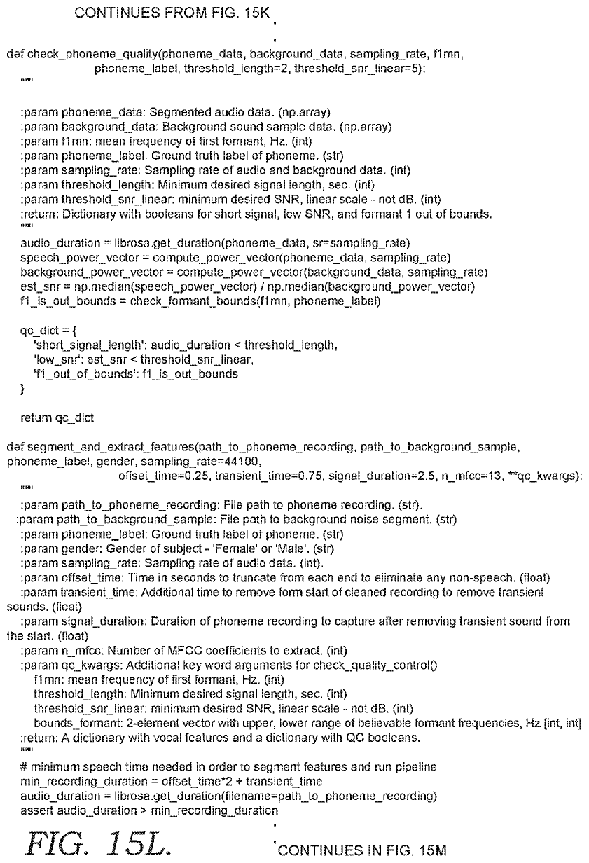

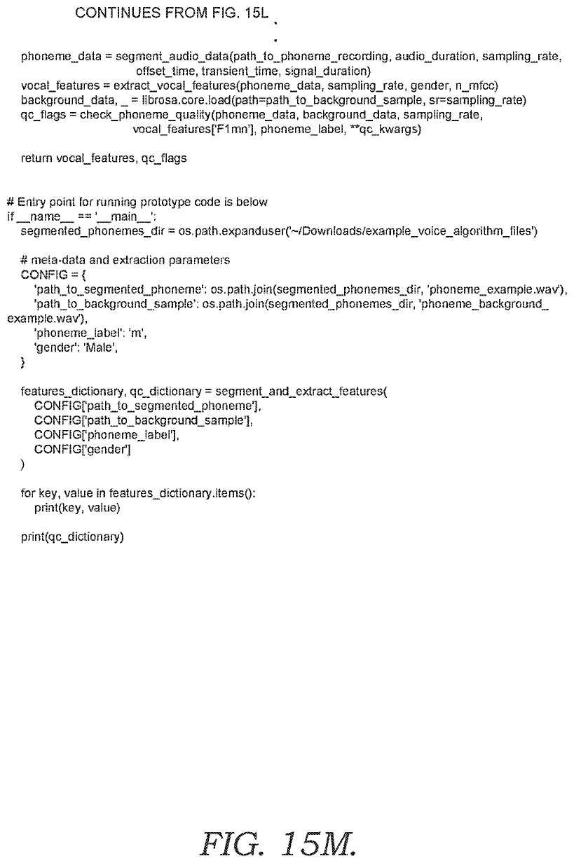

In some embodiments, the specific phoneme features or voice information utilized for monitoring user's respiratory condition may be present in some, but not all, frequency bands of audio data. Accordingly, some embodiments of signal preparation processor 2606 may perform frequency filtering, such as high-pass or band-pass filtering to remove or attenuate frequencies of the audio signal that are less useful, such as lower-frequency background noise. Signal frequency filtering may also improve computational efficiency by reducing an audio sample size and improve processing time for the samples. In one embodiment, signal preparation processor 2606 may apply a band-pass filter of 1.5 to 6.4 kilohertz (kHz). In one exemplary embodiment of a computer program routine provided in FIG. 15A-M, a Butterworth band pass filter is utilized (illustrated in FIG. 15A). In one example, signal preparation processor 26066 may apply a rolling median filter to smooth outliers and normalize features. A rolling-median filter may be applied, using a window of three samples. A z-score may be utilized to normalized the feature values.

Signal preparation processor 2606 may also perform audio normalization to achieve a target signal amplitude level(s), signal-to-noise ratio (SNR) improvement through application of band filters and/or amplifiers, or other signal conditioning or pre-processing. In some embodiments, signal preparation processor 2606 may process the audio data to remove or attenuate background noise, such as background noise determined by background noise analyzer 2603. For example, in some embodiments, signal preparation processor 2606 may perform a noise canceling operation (or otherwise subtract or attenuate the background noise(s) including noise artifacts) using background noise information determined by background noise analyzer 2603.

In user voice monitor 260, sample recording auditor 2608 may generally be responsible for determining whether a sufficient audio sample (or voice sample) is obtained or not. Accordingly, sample recording auditor 2608 may determine that the sample recording has a minimum length of time and/or includes specific voice-related information, such as phonations or other vocal sounds. In some embodiments, sample recording auditor 2608 may apply criteria to check the audio sample based on particular phonemes or phoneme features that are to be detected. In this way, some embodiments of sample recording auditor 2608 may perform phoneme detection on the audio data or operate in conjunction with phoneme segmenter 2610 or other subcomponents of user voice monitor 260. In some embodiments, sample recording auditor 2608 may determine whether an audio sample (or in some instances, a voice sample within an audio recording) satisfies a threshold length of time or not. The threshold length of time may vary based on a particular type of speech-related task that is recorded or may be based on a particular phoneme or phoneme features sought to be obtained from the voice sample, and the extent that those features have already been determined in the current session or timeframe. In one embodiment, in a session to obtain a user voice sample, if a user is prompted (e.g., by user-interaction manager 280) to record a passage reading, sample recording auditor 2608 may determine whether a subsequent voice sample recorded is at least 15 seconds in length or not. Also, in one embodiment, sample recording auditor 2608 may determine whether a particular audio sample includes a sustained phonation for a sufficient duration, such as, at least 4.5 seconds in length or not. Similarly, for embodiments that obtain audio data or voice samples (such as 242) from casual interactions with a user computing device (such as user device 102a), sample recording auditor 2608 may determine that a particular voice sample, to be utilized for further analysis, such as determining phonemes or phoneme features, satisfies a threshold duration and/or includes particular sound(s) or phoneme information. Recordings or voice samples that do not satisfy the auditing criteria (e.g., a minimum threshold duration) may be considered incomplete and may be deleted or not processed further. In some embodiments, sample recording auditor 2608 may provide an indication to the user (or user-interaction manager 280, presentation component 220, or other components of system 200) that a particular sample is incomplete or otherwise deficient, and may further indicate that the user needs to re-record the particular voice sample.

In some embodiments, sample recording auditor 2608 may select a voice sample from among multiple voice samples (which may be received from voice samples 242) that may each represent the same (or similar) voice-related information within a timeframe (i.e., within a session). In some instances, following this selection, the other non-selected samples may be deleted or discarded. For example, where there are multiple complete recordings of the desired phoneme for a given time point or interval (which may have been generated by the user repeating a particular speech-related task), sample recording auditor 2608 may select the recording obtained most recently (the last recorded one) for analysis, which may be done under the assumption that a user re-recorded scripted speech due to technical problems encountered during previous recordings. Alternatively, sample recording auditor 2608 may select a voice sample based on sound parameters, such as one with the lowest amount of noise and/or the highest volume.

Determination of a sufficient voice sample recording for further processing may also include determining there are no noise artifacts, only a minimal amount of noise artifacts exists, and/or that the recording contains at least approximately the correct sounds or indicated instructions are followed. In some embodiments, sample recording auditor 2608 may determine whether the SNR of a voice sample satisfies a maximum allowable SNR or not, such as 20 decibels (dB). For example, sample recording auditor 2608 may determine that the SNR of the recording is greater the threshold of 20 dB and may provide an indication to the user (or to another component of system 200, such as user-interaction manager 280) requesting that a new voice sample be obtained from the user.

Some embodiments of sample recording auditor 2608 may determine whether there are sample sounds corresponding to requested speech-related tasks or not, such as particular sustained phonations (e.g., /a/, /e/, /n/, /m/). In particular, where a voice sample is obtained from a user performing a speech-related task (e.g., “say and hold ‘mmm’ for five seconds”), the voice sample may be checked or audited to determine that the sample includes the sound (or phoneme) that is requested in the task. In some embodiments, this checking operation may utilize automatic speech recognition (ASR) functionality to determine a phoneme in the voice sample and compare the determined phoneme in the sample to the sound or phoneme requested (i.e., the “labeled” phoneme or sound). Where mismatch is determined or where the labeled phoneme or sound is not detected in the sample, sample recording auditor 2608 may provide an indication to the user (or to another component of system 200, such as user-interaction manager 280) so that a correct voice sample may be re-obtained. Additional details of ASR are described in connection with phoneme segmenter 2610 below.

Some embodiments of sample recording auditor 2608 may not necessarily determine the presence of a particular phoneme in an audio sample but may determine that a sustained phoneme or a combination of phonemes is captured in that sample. Sample recording auditor 2608 may also determine whether phonemes have been sustained in the voice sample for a minimum duration or not. In one embodiment, the minimum duration may be 4.5 seconds.

Sample recording auditor 2608 may further perform trimming, cutting, or filtering to remove unnecessary and/or un-useable portions of a voice sample recording. In some embodiments, sample recording auditor 2608 may work with signal preparation processor 2606 to perform such actions. For example, sample recording auditor 2608 may trim a beginning portion and an end portion (e.g., 0.25 seconds) from each recording. Usable portions of a voice sample may include voice-related data that is sufficient for further processing to determine phoneme or feature information. In some embodiments, sample recording auditor 2608 (or voice sample collector 2604 and/or other subcomponents of user voice monitor 260) may prune or trim a voice sample to keep only a portion that is determined to be usable. Similarly, sample recording auditor 2608 may facilitate determining usable portions of audio samples from among multiple samples (such as voice samples 242) that may be obtained within the same timeframe (i.e., within a recording session).

Sample recording auditor 2608 may receive audio sample data from voice samples 242 or from another subcomponent of user voice monitor 260 and, may store the voice sample data it has processed or modified in voice samples 242 or provide the processed or modified voice sample data to another subcomponent of user voice monitor 260. In some instances, such as where a recording is incomplete either after recording or removal of un-useable portions, sample recording auditor 2608 may determine whether a new recording or voice sample needs to be obtained or not and an indication provided to the user, which is described below with respect to user-interaction manger 280.

Phoneme segmenter 2610 may generally be responsible for detecting the presence of individual phonemes in a voice sample and/or determining timing information during which individual phonemes are present in the voice sample. For example, timing information may comprise a beginning time (i.e., start time), a duration, and/or an end time (i.e., stop time) for the occurrence of a phoneme in a voice sample, which may be utilized to facilitate identification and/or isolation of the phoneme for feature analysis. In some instances, the start and stop time information may be referred to as the boundaries of the phoneme. As previously mentioned, voice samples may include recordings (e.g., audio samples) of a user vocalizing sustained individual phonemes or of combinations of phonemes, such as scripted and unscripted speech. For example, a voice sample may be created when a user says a word “spring”, and this voice sample may be segmented into individual phonemes (e.g., /s/, /p/, /r/, /i/ and /ng/). In some instances, voice samples of a sustained individual phoneme may be segmented to isolate the phoneme from the rest of the sample.

In some aspects, phoneme segmenter 2610 may detect phonemes and may further isolate phonemes (e.g., either logically using timing information, which may be utilized as a pointer or a reference to the phoneme in the audio sample, or physically, such as by copying or extracting the phoneme-related data from the audio sample). Phoneme detection by phoneme segmenter 2610 may include determining that a voice sample (or portion of a voice sample) has a particular phoneme or one phoneme in a particular set of phonemes. The voice sample data may be received from voice samples 242 or from another subcomponent of user voice monitor 260. The particular phoneme(s) detected by phoneme segmenter 2610 may be based on the phonemes that are analyzed for the respiratory condition of the user. For example, in some embodiments, phoneme segmenter 2610 may detect whether the sample (or samples) includes phonemes corresponding to /n/, /m/, /e/, and/or /a/, or not. In another embodiment, phoneme segmenter 2610 may determine whether the sample (or samples) includes phonemes corresponding to /a/, /e/, /u/, /ae/, /n/, /m/, and/or /ng/, or not. In other embodiments, phoneme segmenter 2610 may detect other phonemes or sets of phonemes, which may comprise phonemes from any spoken language.

In some embodiments of phoneme segmenter 2610, automatic speech recognition (ASR) (referred to as “voice recognition”) functionality is utilized to determine a phoneme from a portion of the voice sample. The ASR functionality may further utilize one or more acoustic models or speech corpora. In an embodiment, a Hidden Markov Model (HMM) may be utilized in processing a speech signal that corresponds to the user's voice sample to determine a set of one or more likely phonemes. In another embodiment, an artificial neural network (ANN), which is sometimes referred to herein as “neural network”, other acoustic models for ASR, or techniques that use combinations of these models may be utilized. For example, a neural network may be utilized as a pre-processing step of ASR to perform dimensionality reduction or feature transformation prior to application of an HMM. Some embodiments of operations performed by phoneme segmenter 2610 for detecting or identifying phonemes from a voice sample may utilize ASR functionality or acoustic models provided via a speech recognition engine or ASR software toolkit, which may include a software package, a module, or a library for processing speech data. Examples of such speech recognition software tools include Kaldi speech recognition toolkit, available via kaldi-asr.org; CMU Sphinx, developed at Carnegie Mellon University; and Hidden Markov Model Toolkit (HTK), developed at the Cambridge University.

As described herein, in some implementations for obtaining a voice sample, the user may perform a speech-related task, which may be part of an assessment exercise such as a repeat sound exercise described in connection with FIG. 5B. Some of these speech-related tasks may request the user to say and hold a particular sound or phoneme. Additionally or alternatively, a speech-related task may request the user to say and sustain a particular sound or phoneme as long as the user can. Various tasks may be used for different phonemes. For example, in one embodiment, a user may be asked to say and hold “aaaa” (or the /a/ phoneme) as long as the user can but may be asked to say and hold other sounds or phonemes (e.g., /e/, /n/, or /m/) for a pre-determined period of time, such as five seconds. In some embodiments, multiple types of speech-related tasks may be collected for the same phoneme.

The audio sample generated by performing this task may be labeled or otherwise associated with the sound or phoneme that the user is requested to utter. For example, if the user is prompted to say and hold “mmm” for five seconds, then the recorded audio sample may be labeled or associated with the “mmm” sound (or the /m/ phoneme).

In some embodiments, phoneme segmenter 2610 may utilize ASR functionality to determine a particular sound(s) or phoneme in an audio sample, which may be obtained by performing the speech-related task or may be received from user speech obtained via casual interactions with a user device. In these embodiments, once a sound or phoneme of the audio sample is determined, the audio sample (or portion of the sample) may be labeled or associated with the sound or phoneme. In one example embodiment, if phoneme segmenter 2610 determines that the audio sample obtained from the user has the “aaa” sound occurring at a particular portion of the sample, phoneme segmenter 2610 may detect the “aaa” sound (or the /a/ phoneme) and label that portion of the audio sample accordingly (e.g., by associating the label with the audio sample or portion in a database). In another embodiment, phoneme segmenter 2610 may isolate the phoneme to determine the timing or phoneme boundaries in the audio sample.

In some embodiments, phoneme segmenter 2610 may isolate a phoneme by identifying phoneme boundaries or a start time, a duration, and/or a stop time of an interval within the voice sample that captures the phoneme. In some embodiments, phoneme segmenter 2610 first detects the presence of a particular phoneme and then isolates the particular phoneme, such as /n/, /m/, /e/, and /a/ for example. In an alternative embodiment, phoneme segmenter 2610 may detect that particular phonemes are present in the voice sample and isolate all detected phonemes. Some embodiments of phoneme segmenter 2610 may utilize phonetic segmentation or phonetic alignment tools to facilitate determining a time position of a phoneme or phoneme boundary in the audio sample. Examples of such tools are included in functionality provided by the Praat computer software package for speech analysis and phonetics developed at the University of Amsterdam, and/or software modules that operate in conjunction with Praat, such as EasyAlign developed at the University of Geneva for performing phonetic alignment.

In exemplary aspects, phoneme segmenter 2610 may perform automated segmentation by applying thresholds to detected intensity levels in the voice samples. For example, acoustic intensity throughout a recording may be computed, and a threshold for separating background noise from more energetic events in the sample (representing speech events) may be applied. In an embodiment, computation of acoustic intensity may be performed utilizing functions provided by the Praat computer software package for speech analysis and phonetics. FIG. 15A-M illustratively provides one such example using Praat, which is shown using the Parselmouth Python library. A threshold for phoneme segmentation may be determined using Otsu's method, in accordance with an embodiment. In some embodiments, this threshold may be determined for each voice sample such that different thresholds may be determined and applied to different voice samples for the same user. Once the acoustic intensity levels are computed and a threshold is determined, phoneme segmenter 2610 may apply the threshold to the computed intensity levels to detect the presence of a phoneme and may further identify a start time and a stop time corresponding to the beginning and end, respectively, of the detected phoneme. Some embodiments include using manual segmentation on at least some of the voice samples to validate automated segmentation performed by phoneme segmenter 2610.

In some embodiments, gaps within a segment detected as a phoneme may be filled using a morphological “fill” operation. A gap may be filled where the duration of the gap is less than a maximum threshold, such as 0.2 seconds. Additionally, embodiments of phoneme segmenter 2610 may trim one or more portions of the detected phoneme. For example, phoneme segmenter 2610 may trim or disregard an initial duration, such as the first 0.75 seconds, of each detected phoneme to avoid transient effects. Accordingly, the start time of detected phoneme may be changed so that the detected phoneme does not include the first 0.75 seconds. Additionally, in some embodiments, each detected phoneme may be trimmed so that the total duration of phoneme is 2 seconds or other set duration.

In some embodiments, data quality checks may be performed on the segmented phonemes. These data quality checks may be performed by phoneme segmenter 2610 or another component of user voice monitor 260, such as signal preparation processor 2606 and/or sample recording auditor 2608. In one embodiment, a signal-to-noise ratio (SNR) is estimated for each phoneme segment as the ratio of the mean intensity in the detected segment divided by the mean intensity outside the detected segment. Further, a pre-determined segment duration threshold may be applied to determine whether a detected phoneme satisfies a minimum duration or not. Another quality check may include determining a correct number of phonemes by comparing the number of detected phonemes to an expected number of phonemes, which may be based on a prompt(s) triggering a voice sample from the user. For example, in one embodiment, a correct number of phonemes may include three segmented phonemes for sustained nasal consonant recordings and four segmented phonemes for sustained vowel recordings. In an exemplary aspect, a voice sample that has been segmented may be determined as good quality if the correct number of phonemes is found (e.g., three for sustained nasal consonant recordings and four for sustained vowel recordings), the SNR is greater than 9 decibels, and each phoneme has a duration of 2 seconds or greater. In some embodiments, an additional quality check may be performed for vowel voice sample, which may include determining whether the first formant frequency falls within acceptable bounds or not. If it falls within acceptable bounds, the sample is determined to be of good quality. If not, an indication (which may be provided to user-interaction manager 280) is provided that the sample is deficient, incomplete, or that the sample should be re-obtained.

In continuation with user voice monitor 260, acoustic feature extractor 2614 may generally be responsible for extracting (or otherwise determining) features of a phoneme within a voice sample. Features of a phoneme may be extracted from a voice sample at a pre-determined frame rate. In one example, features are extracted at a rate of 10 milliseconds. The extracted features may be utilized for tracking a user's respiratory condition, such as described further with respect to respiratory-condition tracker 270. Examples of acoustic features extracted may include, by way of example and without limitation, data characterizing measures of power and power variability, pitch and pitch variability, a spectral structure, and/or formants.

Further examples of features relating to power and power variability (which may also be referred to as amplitude related features) may include a root-mean-square (RMS) of acoustic power, a shimmer, and power fluctuations in the ⅓-octave band (i.e., third octave band) for each segmented phoneme. In some embodiments, RMS of acoustic power is computed and utilized to normalize data prior to extracting any other acoustic features. Additionally, RMS may be converted to decibels for consideration as a power-related feature itself. Shimmer captures rapid variability in waveform amplitudes measured at glottal pulse intervals. Fluctuations in power within output of ⅓ octave band filter may be computed at various frequencies. In an example embodiment, an extracted feature may indicate the fluctuations in the 200 hertz (Hz) third-octave band, which may be determined by applying a passband frequency of 178-224 Hz.

Further examples of features relating to pitch and pitch variability may include coefficient of variation (COV) of pitch and jitter. To extract the coefficient of variation of pitch, a mean pitch (pitchₘₙ) and a pitch standard deviation (pitch_{sd}) may be determined across each segment, and the coefficient of variation of pitch (pitch_{cov}) may be computed as copitch_{cov}=pitch_{sd}/pitchₘₙ. In some embodiments, particularly where the voice sample is noisy, a coefficient of variation threshold may be applied to ensure that the estimated pitch values are computed for the appropriate frequency for user's voice data. For instance, it may be determined whether the coefficient of variation is below a threshold of 10% of coefficient of variation values or not (determined empirically), and segments in which the value is greater than the threshold may be treated as missing data. Jitter may capture pitch variability on shorter time scales. Jitter may be extracted in the form of local jitter or local absolute jitter. In some aspects, the pitch-related features are extracted from each segment using an auto-correlation method. One example of autocorrelation for determining pitch-related features is provided by the Praat computer software package for speech analysis and phonetics developed at the University of Amsterdam. FIGS. 15E and 15F depict aspects of an example computer programming routine for an embodiment that utilizes the Praat functionality in this manner.

Some embodiments of acoustic feature extractor 2614 (or user voice monitor 260) may perform processing operations to adjust the pitch floor prior to extracting pitch-related features by acoustic feature extractor 2614. For instance, the pitch floor may be increased to 80 Hz for male users and 100 Hz for female users to prevent false pitch detections. Raising the pitch floor may be warranted where low-frequency periodic background noise is present, in accordance with an embodiment. Determination of whether or not to adjust the pitch floor may vary based on a system collecting the voice data, an environment in which the voice data is collected, and/or application settings (e.g., settings 249).

Features relating to spectral structure may include a Harmonics-to-Noise Ratio (HNR, sometimes referred to as “harmonicity”), spectral entropy, spectral contrast, spectral flatness, voice low-to-high ratio (VLHR), mel-frequency cepstral coefficients (MFCCs), cepstral peak prominence (CPP), percentage or proportion of voiced (or unvoiced) frames, and linear predictive coefficients (LPCs). HNR or harmonicity is a ratio of power in harmonic components to power in non-harmonic components and represents a degree of acoustic periodicity. An example of determining HNR is shown in the computer programming routine of FIG. 15E, which utilizes functionality provided by the Praat computer software package for determining harmonicity. Spectral entropy indicates the entropy of a spectrum in a particular frequency band. Spectral contrast may be determined by sorting power spectrum values by intensity in a particular frequency band and computing a ratio of a highest quartile of values (peaks) to a lowest quartile of values (troughs) in the frequency band. Spectral flatness may be determined by computing the ratio of the geometric mean to the arithmetic mean of spectrum values in a given frequency band. Spectral entropy, spectral contrast, and spectral flatness each may be computed for specific frequency bands. In one embodiment, spectral entropy is determined at 1.5-2.5 kilohertz (kHz) and 1.6-3.2 kHz; spectral flatness is determined at 1.5-2.5 kHz; spectral contrast is determined at 1.6 to 3.2 kHz and 3.2-6.4 kHz.

VLHR may be determined by computing a ratio of integrated low-to-high frequency energy. In one embodiment, the separation between low and high frequencies is fixed at 600 Hz. As such, the feature may be denoted as VLHR600.

Mel-frequency cepstral coefficients (MFCCs) represent a discrete cosine transform of a scaled power spectrum and MFCCs collectively make up a mel-frequency cepstrum (MFC). MFCCs are typically sensitive to changes in the spectrum and robust to environmental noise. In exemplary aspects, mean MFCC values and standard deviation MFCC values are determined. In one embodiment, means values are determined for mel-frequency cepstral coefficients MFCC6 and MFCC8 and standard deviation values are determined for mel-frequency cepstral coefficients MFCC1, MFCC2, MFCC3, MFCC8, MFCC9, MFCC10, MFCC11, and MFCC12.

Voicing refers to the periodicity in a recorded phonation, and some aspects of the disclosure include determining a percentage, proportion, or ratio of frames of a phonation recording that are voiced. Alternatively, this feature may be determined using unvoiced frames. In some instances of determining voiced (or unvoiced) frames, a predetermined pitch threshold may be applied so that the percentage of voiced or unvoiced frames is being termed for frames that have suspected speech. In some embodiments, the percentage or proportion of voiced (or unvoiced) frames may be determined using the Praat computer software package toolkit for voice processing.

Other features extracted or determined by acoustic feature extractor 2614 may relate to one or more acoustic formants, which represent resonances of the vocal tract. In particular, for a phoneme of a voice sample, a mean formant frequency and a standard deviation of formant bandwidth may be computed for one or more formants In exemplary aspects, mean formant frequency and standard deviation of formant bandwidth are computed for formant 1 (denoted as F1); however, it is contemplated that additional or alternatives may be utilized, such as formants 2 and 3 (denoted as F2 and F3). In some aspects, formant features may operate as a data quality control by facilitating automatic checks, which may be performed by sample recording auditor 2608, to ensure that users are pronouncing sounds correctly.

It is contemplated that in some embodiments, each of the described acoustic features may be extracted or determined for different phonemes. For instance, in one embodiment, 23 of the above features (not including RMS for amplitude) are determined for seven phonemes (/a/, /e/, /i/, /u/, /ae/, /n/, /m/ and /ng/), resulting in 161 unique phoneme features. Some embodiments of the present disclosure may include identifying or selecting a set of features for further analysis. For example, one embodiment may include determining all 161 features from one or more voice samples, or reference voice data, and selecting or otherwise determining particular features considered to be relevant to monitoring user's respiratory infection condition.

Additionally, one or more of these acoustic features may be extracted from voice samples from only certain types of speech-related tasks. For example, the above described features may be determined for phonemes extracted from phonations of a pre-determined duration. One or more of these above-described features may be determined for phonations extracted from a user reading a passage. In some embodiments, other features may be extracted from certain types of speech-related tasks. For example, in example aspects, a maximum phonation time, which may be used as a measure of respiratory capacity, may be determined from sustained phonation voice samples where a user holds a sound as long as possible. As used herein, maximum phonation time refers to the duration that a user sustains a particular phonation.

Further, in some embodiments, a change in amplitude within a sustained phonation may also be determined for these types of voice samples. In some example embodiments, other acoustic features are determined from a passage voice sample. For example, from a recording or monitoring of a user reading a passage, a speaking rate an average pause length, a pause count, and/or a global SNR may be determined. The speaking rate may be determined as the number of syllables or words per second. Pause length may refer to pauses in a user's speech that are at least a predetermined minimum duration, such as 200 milliseconds. In some aspects, pauses used to determine an average pause length and/or pause count may be determined by utilizing an automated speech-to-text algorithm to generate text from user's voice sample, determine timestamps for when a user starts a word and when a user finishes a word, and, using the timestamps, determining the durations between words. The global SNR may be the signal-to-noise ratio over the recording that includes nonspoken time.

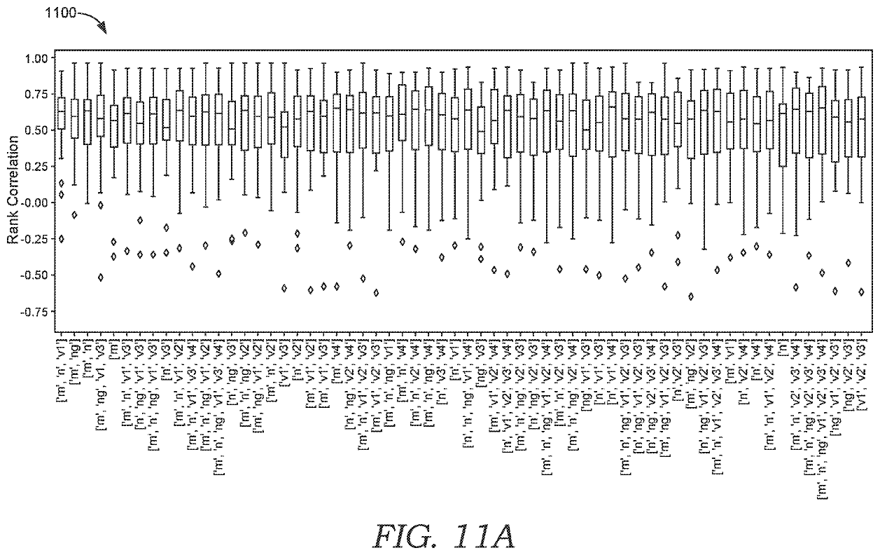

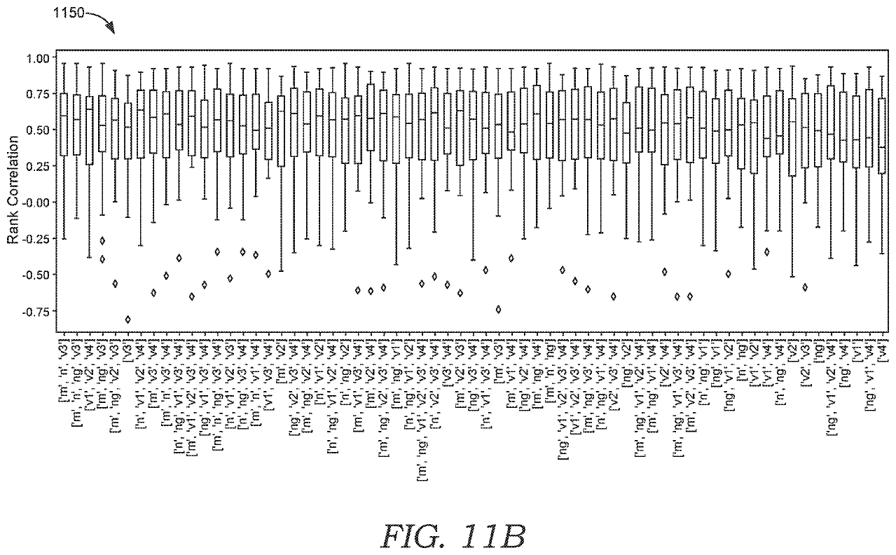

It is further contemplated that particular features or combinations of features are more suitable for monitoring certain types of respiratory infections than others. Embodiments of feature selection may include identifying possible feature combinations, calculating a distance metric between feature sets or vectors for different days, and correlating the distance metric for self-reported ratings for respiratory symptom. In one example, principal component analysis (PCA) is utilized to compute the first six principal components for possible phoneme combinations (illustrated in, e.g., FIGS. 11A and 11B for example phoneme combinations) and calculate a distance metric, such as the Euclidean distance between vectors representing the acoustic features for the combination of phonemes across each pair of days for which voice data is collected. Spearman's rank correlation may be computed between the distance metric for each day relative to a final day representing a well state and self-reported symptom ratings.

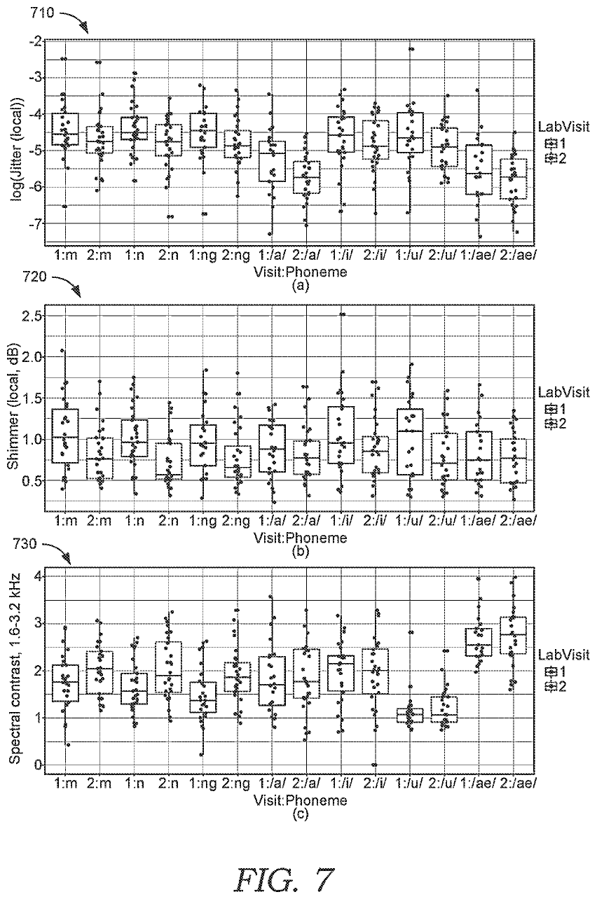

Further, in some embodiments, unsupervised feature selection is also performed by applying sparse PCA to further reduce dimensionality of the dataset. Alternatively, in some embodiments, Linear Discriminant Analysis (LCA) may be utilized to reduce dimensionality. In some embodiments, features (specifically, phoneme and feature combination) in the top quantity of principal components (determined empirically) with a non-zero weight may be selected for further analysis. Aspects of feature selection are discussed further in conjunction with FIGS. 7-14.

In exemplary aspects, a representative phoneme feature set, determined from feature selection described in connection with FIGS. 7-14, comprises 32 phoneme features including 12 features of the /n/ phoneme, 12 features of the /m/ phoneme, and 8 features of the /a/ phoneme. These example 32 features are listed in the table below.

| Phoneme | Acoustic Feature |
|---|---|
| /m/ | Harmonicity |
| | Pitch interquartile range (IQR) (LG) |
| | F1 bandwidth standard deviation |
| | Spectral Entropy: 1.5-2.5 kHz, 1.6-3.2 kHz |
| | Standard Deviation of MFCC (LG): MFCC 2, 10 |
| | Spectral Flatness 1.5-2.5 kHz |
| | Mean MFCC: MFCC 8 |
| | Shimmer (local, dB) |
| | Spectral Contrast 3.2-6.4 kHz (LG) |
| | 200 Hz TOB (third-octave band) standard deviation (LG) |
| /n/ | Harmonicity |
| | F1 bandwidth standard deviation |
| | Pitch interquartile range (IQR) (LG) |
| | Spectral Entropy: 1.5-2.5 kHz, 1.6-3.2 kHz |
| | Spectral Flatness: 1.5-2.5 kHz |
| | Standard Deviation of MFCC (LG): MFCC 1, 2, 3, 11 |
| | Mean MFCC: MFCC 8 |
| | Spectral Contrast: 1.6-3.2 kHz (LG) |
| /a/ | F1 bandwidth standard deviation |
| | Pitch interquartile range (IQR) (LG) |
| | Spectral Entropy: 1.6-3.2 kHz |
| | Jitter (local) (LG) |
| | Standard Deviation of MFCC (LG): MFCC 9, 12 |
| | Mean MFCC: MFCC 6 |
| | Spectral Contrast 3.2-6.4 kHz (LG) |

As indicated in the table above, values for one or more features may be transformed by acoustic feature extractor 2614 for normality. For instance, a log transformation (denoted as LG) may be applied to a subset of features. Other features may not include a transformation. Further, although not included in the above table, it is contemplated that other transformations, such as a square root transform (SRT) may be applied. In one embodiment, feature selection includes selecting transformations for various one of more features. In one example, different types of transformations, such as SRT, LG, or no transformations, are tested on one or more features, and the Shapiro-Wilk test may be used to select the transformation type that gave the most normally-distributed data for that particular feature.

In some embodiments, acoustic feature extractor 2614, phoneme segmenter 2610, or other subcomponents of user voice monitor 260 may determine phonemes or extract features for phoneme utilizing voice-phoneme extraction logic 233 (as shown in storage 250 in FIG. 2). Voice-phoneme extraction logic 233 may include instructions, rules, conditions, associations, machine learning models, or other criteria for identifying and extracting acoustic feature values from acoustic data corresponding to the segment phonemes. In some embodiments, voice-phoneme extraction logic 233 utilizes ASR functionality, acoustic models, or related functionality described in connection with phoneme segmenter 2610. For example, various classification models or software tools (e.g., HMM, neural network models, and other software tools described previously) may be utilized to identify a particular phoneme in an audio sample and determine corresponding acoustic features. One example embodiment of acoustic feature extractor 2614 or voice-phoneme extraction logic 233 may include or utilize functionality provided in the Praat computer software package for speech analysis and phonetics. Aspects of one such embodiment, comprising a computer program routine, are illustratively provided in FIGS. 15A-M, which are shown using the Parselmouth Python library for accessing the Praat software package.

After determining the phoneme features, acoustic feature extractor 2614 may determine a phoneme feature set, which may comprise a phoneme feature vector (or a set of phoneme feature vectors) for the phonemes determined from the user voice sample(s) corresponding to a recording session or a timeframe. For example, a user may provide voice samples twice a day (e.g., a morning session and an evening session), and each session may correspond to a phoneme feature vector or a set of vectors representing features extracted or determined from the phonemes detected from the voice sample captured during that session. The phoneme feature set may be stored in individual record 240 associated with the user, such as phoneme feature vectors 244, and may be stored or otherwise associated with date-time information corresponding to the date or time the voice samples, used to determine the phoneme features, are obtained.

In some instances, the terms “feature set” and “feature vector” may be used interchangeably herein. For example, in order to facilitate performing a comparison between two feature sets, member features of the set may be considered as a feature vector so that a distance measurement may be determined between corresponding features in each vector (i.e. a feature vector comparison), or to facilitate applying other operations to the features. In some embodiments, phoneme feature vectors 244 may be normalized. In some instances, a feature vector may be a multiple dimensional vector, where each phoneme has dimensions representing the features. In some embodiments, multidimensional vectors may be flattened, such as prior to determining a comparison between two feature vectors, as described in connection with respiratory-condition tracker 270.

In addition to determining acoustic features, some embodiments of user voice monitor 260 may include contextual information determiner 2616 to determine contextual information related to the voice samples from which features are determined. The contextual information may indicate, for example, conditions at the time of the voice sample recording. In example embodiments, contextual information determiner 2616 may determine a date and/or time of the recording (i.e., a timestamp) or duration of the recording that may be stored or otherwise associated with the phoneme feature vector(s) generated by acoustic feature extractor 2614. Information determined by contextual information determiner 2616 may be relevant to tracking a user's respiratory condition in addition to the extracted acoustic features. For example, contextual information determiner 2616 may also determine the particular time of day (e.g., morning, afternoon or evening) that the voice sample is obtained and/or user location from which environmental or atmospheric-related information (e.g., weather, humidity, and/or pollution levels) may be determined. In one embodiment, the duration of a voice sample may also be used to track the user's respiratory condition. For example, a user may be asked to say and hold the sound “aaaa” (i.e., phoneme /a/) for as long as the user can, and a duration metric measuring the duration that the user was able to hold the sound may be used to determine the user's respiratory condition.

In some embodiments, contextual information determiner 2616 may determine or receive physiological information about the user, which may be associated with the timeframe a voice sample is obtained. For example, the user may provide information about symptoms that he is or she is feeling, as shown and described in the embodiments depicted in FIGS. 4D, 5D and 5E. In some instances, contextual information determiner 2616 may operate in conjunction with user-interaction manager 280 to obtain symptom data, as described below. In some embodiments, contextual information determiner 2616 may receive physiological data, such as a body temperature or blood oxygen level on a wearable user device (e.g., a fitness tracker), from a user's profile/health data (EHR) 241 or a sensor (such as 103 of FIG. 1).

In some embodiments, contextual information determiner 2616 may determine whether the user is on a medication or not and/or if the user has taken the medication. This determination may be based on the user providing an explicit signal, such as selecting an indicator on an digital application, signifying that the user has taken a medicine or responding to a prompt from a smart device asking the user if he or she took his or her medicine, or may be provided by another sensor, such as a smart pillbox or a medicine container, or from another user, such as a user's caretaker. In some embodiments, contextual information determiner 2616 may determine that the user is on medication based on information provided by the user, a doctor or a healthcare provider, or a caregiver, by accessing the user's electronic health record (EHR) 241, emails or messaging indicating prescriptions or purchases, and/or purchase information. For example, a user or a care provider may specify a particular medicine that the user is taking or a treatment regimen via a digital application, such as an example respiratory-infection monitor app 5101 described in conjunction with FIG. 5D.

Contextual information determiner 2616 may further determine a user's geographic region (for example, by a location sensor on the user device or the user's input of location information, such as a zip code). In some embodiments, contextual information determiner 2616 may further determine the extent of a particular virus or bacteria known to cause a respiratory infection, such as influenza or COVID-19, which is present in the user's geographic region. Such information may be available from government or healthcare websites or web portals, such as those operated by the U.S. Centers for Disease Control and Prevention (CDC), the World Health Organization (WHO), state health departments, or national health agencies.

Information determined by contextual information determiner 2616 may be stored in individual record 240, and in some embodiments, the information may be stored in a relational database, such that the contextual information is associated with a particular voice sample or the particular phoneme feature vector(s) determined from the voice sample, which also may be stored in individual record 240.

As described above, user voice monitor 260 may generally be responsible for obtaining relevant acoustic information from an audio sample of the user's voice. Collection of this data may involve directing interactions with a user. Accordingly, embodiments of system 200 may further include user-interaction manager 280 to facilitate the collection of user data, including obtaining voice samples and/or user symptom information. As such, embodiments of user-interaction manager 280 may include a user-instruction generator 282, self-reporting tools 284, and a user-input response generator 286. User-interaction manager 280 may work in conjunction with user voice monitor 260 (or one or more of its subcomponents), presentation component 220 and, in some embodiments, a self-reporting data evaluator 276 as described later herein.

User-instruction generator 282 may generally be responsible for guiding a user to provide voice samples. User-instruction generator 282 may provide (e.g., facilitate displaying via a graphic user interface, such as shown in the example of FIG. 5A or speaking via an audio or voice user interface, such as shown in the example interaction of FIG. 4C) a procedure for capturing the voice data to the user. Among other things, user-instruction generator 282 may read and/or speak instructions 231 for the user (e.g., “Please say ‘aaa’ for 5 seconds.”). The instructions 231 may be pre-programmed and specific to the phonemes, voice-related data, or other user-information that is sought from the user. In some instances, instructions 231 may be determined by a clinician or a caregiver of the user. In this way, instructions 231 may be specific to the user (e.g., as part of treatment as a patient) and/or specific to a respiratory infection or a medication, in accordance with some embodiments. Alternatively, or in addition, instructions 231 may be automatically generated (e.g., synthesized or assembled). For example, instructions 231 requesting a specific phoneme may be generated based on determining that feature information about the specific phoneme is needed or helpful for determining the user's respiratory condition. Similarly, a set of pre-determined instructions 231 or operations may be provided (e.g., from a clinician, a caregiver, or programmed into a decision support application, such as 105a or 105b) and used to assemble specific or tailored instructions for the user.

The pre-programmed or generated instructions 231 may relate to performing a specific speech-related task, such as speaking a particular phoneme for a set duration, speaking and holding a particular phoneme for as long as possible, speaking particular words or combinations of words, or reading aloud a passage. In some embodiments in which reading aloud a passage is requested of the user, the text of the passage may be provided to the user so that the user may read the provided passage aloud. Additionally or alternatively, portions of the passage may be audibly output to the user so that a user may repeat the audible passages without reading text. In one embodiment, a user is requested to say aloud (either by reading written text or repeating spoken instructions) a pre-determined phonetically-balanced passage, such as the rainbow passage, and may be requested to read a certain portion of the passage, such as five lines of the of the rainbow passage. In some instances, the user may be give a pre-determined amount of time, such as two minutes, to complete reading the passage.

In some embodiments, instructions 231 may provide sample sounds for the phonemes that are instructed to be provided by the user. In some embodiments, user-instruction generator 282 may provide instructions 231 only for phonemes or sounds that are sought for the respiratory-condition analysis, which may comprise providing only a portion of the instructions 231. For example, where user voice monitor 260 has not yet obtained a voice sample that includes a particular phoneme for a given timeframe, user-instruction generator 282 may provide instructions 231 to facilitate obtaining a voice sample with that phoneme information. Additional examples showing instructions 231 that may be provided by user-instruction generator 282 (or user-interaction manager 280) are depicted and further described in connection with FIGS. 4A, 4B and 5B.

Some embodiments of user-instruction generator 282 may provide instructions 231 tailored to a particular user. As such, user-instruction generator 282 may generate instructions 231 based on the particular user's health condition, a clinician's orders, prescriptions, or recommendations for the user, the user's demographic or EHR information (e.g., if a user is determined to be a smoker, the instructions are modified), or based on previously captured voice/phoneme information from the user. For example, analysis of previous phonemes provided by the user may indicate particular phonemes showing more changes during all or part of a respiratory infection (e.g., during recovery). Additionally, or alternatively, it may be determined that the user has a respiratory condition that is more easily detected or tracked by some phoneme features over other features. In these instances, an embodiment of user-instruction generator 282 may instruct the user to capture additional samples of that phoneme(s) of interest or may generate or modify instructions 231 to remove (or not to provide) instructions for obtaining voice samples with phonemes that are less useful for the particular user. In some embodiments of user-instruction generator 282, instructions 231 may be modified based on previous determinations of the user's respiratory condition (e.g., whether or not the user is sick or is recovering).

Self-reporting tools 284 may generally be responsible for guiding a user to provide data that may be related to their respiratory condition and, other contextual information. Self-reporting tools 284 may interface with self-reporting data evaluator 276 and data collection component 210. Some embodiments of self-reporting tools 284 may operate in conjunction with user-instruction generator 282 to provide instructions 231 to guide a user to provide user-related data. For example, self-reporting tools 284 may utilize instructions 231 to prompt the user to provide information about symptoms the user is experiencing relating to a respiratory condition. In one embodiment, self-reporting tools 284 may prompt a user to rate a severity of each symptom within a set of symptoms, which may be congestion-related or non-congestion related. Additionally, or alternatively, self-reporting tools 284 may utilize instructions 231 or ask the user to provide information about the health of that user or how he is feeling generally. In one embodiment, self-reporting tools 284 may prompt the user to indicate a severity of post-nasal discharge, nasal obstruction, runny nose, thick nasal discharge with mucus, cough, sore throat, and need to blow nose. In some embodiments, self-reporting tools 284 may comprise user-interface elements to facilitate prompting the user or receiving data from the user. For example, aspects of GUIs for providing self-reporting tools 284 are depicted in FIGS. 5D and 5E. Example user-interactions showing aspects of a voice user interface (VUI) for providing self-reporting tools 284 are depicted in FIGS. 4D, 4E, and 4F.

In some embodiments, self-reporting tools 284, utilizing instructions 231, may prompt a user to provide symptom or general condition input multiple times a day, and the input requested may vary based on the time of day. In some embodiments, the input times may correspond to timeframes or sessions in which user voice sample is obtained. In one example, self-reporting tools 284 may prompt the user to rate the perceived severity of 19 symptoms in the morning and 16 symptoms in the evening. Additionally, or alternatively, self-reporting tools 284 may prompt the user to answer four sleep-related questions in the morning and one end-of-day tiredness question in the evening. The table below shows an example list of prompts for user input that may be determined by self-reporting tools 284, utilizing instructions 231 and output by self-reporting tools 284 or other subcomponent of user-interaction manager 280.

| Question | Possible values | Morning | Evening |
|---|---|---|---|
| How well do you fell this | 0 to 5 | x | |
| morning? | | | |
| Did you have difficulties | 0 to 5 | x | |
| falling asleep last night? | | | |
| Did you have a restless | 0 to 5 | x | |
| night? | | | |
| Do you feel like you have | 0 to 5 | x | |
| a lack of a good night's | | | |
| sleep? | | | |
| Did you wake up tired? | 0 to 5 | x | |
| Do you feel the need to | 0 to 5 | x | x |
| blow your nose? | | | |
| Have you been sneezing? | 0 to 5 | x | x |
| Do you have a runny | 0 to 5 | x | x |
| nose? | | | |
| Do you feel like you have | 0 to 5 | x | x |
| any nasal obstructions | | | |
| (blocked nose)? | | | |
| Have you experienced any | 0 to 5 | x | x |
| loss of smell or taste? | | | |
| Have you been coughing? | 0 to 5 | x | x |
| Have you experienced any | 0 to 5 | x | x |
| post-nasal discharge? | | | |
| Have you experienced any | 0 to 5 | x | x |
| thick nasal discharge | | | |
| (thick mucus)? | | | |
| Have you had a sore | 0 to 5 | x | x |
| throat? | | | |
| New or increased cough | 0 to 5 | x | x |
| New or increased nasal | 0 to 5 | x | x |
| congestion | | | |
| New or increased nasal | 0 to 5 | x | x |
| discharge | | | |
| New or increased | 0 to 5 | x | x |
| wheezing | | | |
| New or increased | 0 to 5 | x | x |
| shortness of breath | | | |
| Select your worst | Multi choice | x | x |
| symptoms (up to 5) | | | |
| How well did you feel | 0 to 5 | | x |
| today? | | | |

In some embodiments, self-reporting tools 284 may provide follow-up questions or provide follow-up prompts based on the user's detected phoneme features (i.e., based on a suspected respiratory condition), previously captured phoneme data, and/or other self-reported input. In one exemplary embodiment, if an analysis of phoneme features indicates that the user may be developing a respiratory infection or still recovering from a respiratory infection, self-reporting tools 284 may facilitate prompting the user to report symptoms. For example, self-reporting tools 284, which may utilize instructions 231 and/or operate in conjunction with user-interaction manager 280, may ask the user about (or display a request soliciting) the user's symptoms. In this embodiment, the user may be asked questions regarding how the user feels, such as “Do you feel congested?”. In a similar example, if the user reports that the user is congested or has a particular symptom, then self-reporting tools 284 may follow up by asking “How congested are you, on a scale of 1-10?” or prompting the user to provide this follow-up detail.

In some embodiments, self-reporting tools 284 may comprise a functionality enabling a user to communicatively couple a wearable device, a health-monitor, or a physiological sensor to facilitate automatic collection of the user's physiological data. In one such embodiment, the data may be received by contextual information determiner 2616 or other component of system 200 and may be stored in individual record 240. In some embodiments, as described previously, this information received from self-reporting tools 284 may be stored in a relational database, such that it is associated with a particular voice sample or the particular phoneme feature vector(s) determined from the voice sample obtained from a session. In some embodiments, based on the received physiological data, self-reporting tools 284 may prompt or request the user to self-report symptom information, as described above.

User-input response generator 286 may generally be responsible for providing feedback to the user, in accordance with various embodiments. In one such embodiment, user-input response generator 286 may analyze user's input of user data, such as speech or voice recordings, and may operate in conjunction with user-instruction generator 282 and/or sample recording auditor 2608 to provide feedback to the user based on the user's input. In one embodiment, user-input response generator 286 may analyze a user's response to determine whether the user provided a good voice sample or not and then provide an indication of that determination to the user. For instance, a green light, a checkmark, a smiley face, thumbs up, a bell or a chirp sound, or similar indicator may be provided to the user to indicate that the recorded sample is good. Likewise, a red light, a frowny face, a buzzer, or similar indicator may be provided to inform the user that the sample was incomplete or defective. In some embodiments, user-input response generator 286 may determine if the user failed to comply with the instructions 231 from user-instruction generator 282. Some embodiments of user-input response generator 286 may invoke a chatbot software agent to provide in-context help or assistance to the user if an issue is detected.

Embodiments of user-input response generator 286 may inform the user if a sound level or other acoustic properties of a previous voice sample is insufficient, there is too much background noise, or the sound being recorded in the sample is not long enough. For example, after the user provides an initial voice sample, user-input response generator 286 may output “I didn't hear that; let's try again. Please ‘say aaaa’ for 5 seconds.”. In one embodiment, user-input response generator 286 may indicate a level of loudness that the user should try to achieve during recording and/or provide feedback to the user on whether the voice sample is acceptable or not, which may be determined in accordance with sample recording auditor 2608.

In some embodiments, user-input response generator 286 may utilize aspects of a user interface to provide feedback to the user regarding sound level, background noise, or timing duration of obtaining a voice sample. For instance, a visual or audio countdown clock or timer may be used to signal to the user when to start or stop speaking for recording a voice sample. One embodiment of a timer is depicted as a GUI element 5122 in FIG. 5A. A similar example for providing user-input response is depicted as GUI element 5222 in FIG. 5B, which includes a timer and an indicator of background noise. Other examples (not shown) may include GUI elements for audio input level(s), background noise, color-changing the words or a ball that hops along the words that a user is reading as the words are spoken, or a similar audio or visual indicator.

User-input response generator 286 may provide the user with an indication of progress of a particular speech-related task (e.g., vocalizing a phonation) or a voice session. For instance, as described above, user-input response generator 286 may count (either displayed on a graphic user interface or through an audio user interface) the seconds when a user provides a sustained phonation or may tell the user when to start and/or stop. Some embodiments of user-input response generator 286 (or user-instruction generator 282) may provide an indication regarding the speech-related tasks to be completed or the speech-related tasks that have already been completed for a particular session, a timeframe, or a day.

As described previously, some embodiments of user-input response generator 286 may generate visual indicators for the user, such that the user may see feedback of the provided voice sample, such as, for example, indicators regarding a volume level of a sample, the sample is acceptable or not, and/or the sample is correctly captured or not.

Utilizing voice information collected and determined by user voice monitor 260 (alone or in conjunction with user-interaction manager 280) or respiratory-condition tracker 270 may determine information about a user's respiratory condition and/or a prediction about the user's future respiratory condition. In one embodiment, respiratory-condition tracker 270 may receive a phoneme feature set (e.g., one or more phoneme feature vectors) associated with a particular time or timeframe and which may be timestamped with the date and/or time information. For instance, the phoneme feature set may be received from user voice monitor 260 or from individual record 240 associated with the user, such as phoneme feature vectors 244. The time information associated with a phoneme feature set may correspond to a date and/or time that the voice sample(s) (or voice-related data) used to determine the phoneme feature set is obtained from the user, as described herein. Respiratory-condition tracker 270 may also receive contextual information related to the audio recordings or voice samples from which the phoneme features are determined, which also may be received from individual record 240 and/or user voice monitor 260 (or specifically, contextual information determiner 2616). Embodiments of respiratory-condition tracker 270 may utilize one or more classifiers to generate a score or determination of a user's likely present respiratory condition based on phoneme feature sets (vectors) for multiple times and, in some embodiments, contextual information. Additionally, or alternatively, respiratory-condition tracker 270 may utilize a predictor model to forecast the user's likely future respiratory condition. Embodiments of respiratory-condition tracker 270 may include a feature vector time series assembler 272, a phoneme features comparer 274, self-reporting data evaluator 276, and a respiratory condition inference engine 278.

Feature vector time series assembler 272 may be employed for assembling a time series of successive phoneme feature vectors (or feature sets) for a user. The time series may be assembled in chronological or reverse-chronological order according to the time information (or timestamps) associated with the feature vectors. In some embodiments, the time series may include all of the phoneme feature vectors generated for collected voice samples for the user or individual, phoneme feature vectors generated for samples collected within a time interval in which the individual is sick (i.e., has a respiratory infection), or phoneme feature vectors associated with times within a set or pre-determined time interval, such as the past 3-5 weeks, past two weeks, or past week, for example. In other embodiments, the time series includes only two feature vectors. In one such embodiment, a first phoneme feature vector of the time series may be associated with a recent time period or instance according to a corresponding timestamp and, thus, represent information about a user's current respiratory condition, while the second feature vector may be associated with an earlier time period or instance. In some embodiments, the earlier time period corresponds to a time interval when the user's respiratory condition is different (i.e., a time when the user was sick or healthy) from the recent time period or instance.

Further, phoneme features comparer 274 may generally be responsible for determining differences in phoneme feature vectors 244 (or differences in the values of features in different feature sets) for the user. Phoneme features comparer 274 may determine differences by comparing two or more phoneme feature vectors. For instance, a comparison may be performed between phoneme feature vectors 244 associated with any two different time instances or periods, or between feature vector(s) associated with a recent time period or instance and feature vector(s) associated with an earlier time period or instance. Each compared phoneme feature set (or vector) may be associated with different time periods or instances, such that the comparison by phoneme features comparer 274 may provide information regarding changes in the features (representing changes in the user's respiratory condition) across different time periods or instances. In some embodiments, it is contemplated that two or more feature vectors to be compared may have the same duration or that each vector has corresponding features (i.e., same dimensions) for a comparison. In some instances, only a portion of the feature vector (or a subset of features) may be compared. In one embodiment, a plurality of feature vectors, which may include three or more vectors, each associated with a different time period or instance, may be utilized by phoneme features comparer 274 to perform an analysis characterizing feature changes over a time frame spanning different time periods or instances. For example, the analysis may comprise determining a rate of change, regression or curve fitting, cluster analysis, discriminant analysis, or other analysis. As described previously, although the terms “feature set” and “feature vector” may be used interchangeably herein to facilitate performing a comparison between feature sets, individual features of a feature set may be considered as a feature vector.

In some embodiments, a comparison may be performed between the feature vector(s) of a recent time period or instance (e.g., feature vector(s) determined from the most recently obtained voice sample(s)) and an average or composite of feature vectors corresponding to multiple earlier time periods or instances (e.g., a boxcar moving average based on multiple prior feature vectors or voice samples). In some instances, the average may consider up to a maximum number of feature vectors associated with prior time periods or instances for the user (e.g., the average from feature vectors corresponding to 10 prior sessions of obtaining voice samples) or feature vectors from a pre-determined, earlier time interval, such as the past week or two weeks. Phoneme features comparer 274 may alternatively, or additionally, compare user's feature vector(s) for a recent time interval to a phoneme-features baseline, which, as further described herein, may be based on the user or other users such as a population at large or other users similar to the monitored user (e.g., a cohort having a similar respiratory condition or other similarity to the monitored user). Further, in some instances, the comparison may utilize statistical information about the baseline (or about the feature sets, in embodiments not utilizing the baseline), such as statistical variance or standard deviation of the feature set(s) corresponding to the baseline (or corresponding to the feature set(s)). Employing an average, and in particular a rolling or moving average, may be considered, in some embodiments, to operate as a smoothing function on the prior feature vectors (i.e., feature vectors corresponding to voice samples obtained from earlier time periods or instances). In this way, variations in voice-related data not accounting for respiratory infection that may occur among the earlier samples may be minimized (e.g., whether the voice sample is obtained in the morning when the user first woke up or not versus the end of a long day versus a time after the user had been cheering or singing loudly). It is also contemplated that some embodiments of phoneme features comparer 274 may compare an average of recent feature vectors to an average of earlier feature vectors or to feature vector(s) associated with a single, earlier time period or instance. Similarly, a statistical variance may be determined among the feature values (or portion of feature values) of recent features and compared against the variance of earlier feature values (or their portion).

Some embodiments of phoneme features comparer 274 may utilize phoneme-features comparison logic 235 to determine a comparison of phoneme feature vectors. Phoneme-features comparison logic 235 may comprise computer instructions (e.g., functions, routines, programs, libraries, or the like) and may include, without limitation, one or more rules, conditions, processes, models or other logic for performing a comparison of features or feature vectors, or for facilitating a comparison or processing a comparison for interpretation. In some embodiments, phoneme-features comparison logic 235 is utilized by phoneme features comparer 274 to compute a distance metric or difference measurement of phoneme feature vectors. In exemplary aspects, the distance measurement may be regarded as quantifying change in the acoustic feature space of voice information over a passage of time for a user. In this way, changes in user's respiratory condition may be observed and quantified based on the quantifiable changes detected in the acoustic feature space (e.g., phoneme features) between two or more times in which voice information for the user is obtained. In one embodiment, phoneme features comparer 274 may determine a Euclidian measurement or L2 distance for two feature vectors (or averages of feature vectors) to determine a distance measurement. In some instances, phoneme-features comparison logic 235 may include logic for performing flattening in the case of multi-dimensional vectors, normalization, or other processing operations, prior to or as part of a comparison operation. In some embodiments, phoneme-features comparison logic 235 may include logic for performing other distance metrics (e.g., Manhattan distance). For example, the Mahalanobis distance may be utilized to determine distance between a recent feature vector and a set of feature vectors associated with earlier time periods or instances. In some embodiments, a Levenshtein distance may be determined, such as for implementations comparing the user reading aloud a passage. For example, according to an embodiment, a speech-to-text algorithm may be utilized to generate text from the user's recitation of the passage. A time series of one or more entries may be determined comprising the syllables or words of the passage and a corresponding timestamp of when the user read those words. The time series (or timestamp) information may be used to generate a feature vector (or otherwise may be used as features) for the comparison (e.g., using the a Levenshtein distance algorithm) to a baseline feature vector, determined in a similar manner.

In some embodiments, a phoneme feature difference (or distance metric) may be determined for multiple pairs of times for an individual. For example, a distance may be computed between phoneme feature vector(s) from the most recent day to phoneme feature vector(s) from a day previous to the most recent one, and/or a distance may be computed between phoneme feature vector(s) from the most recent day to phoneme feature vector(s) from samples collected a week ago or to phoneme feature vector representing a baseline. Further, in some embodiments, different types of distance measurements for different phoneme feature vectors or features may be computed.

In some embodiments, a phoneme feature difference (or distance metric) may indicate a difference of a particular acoustic feature over time period or instance. For example, phoneme features comparer 274 may compute a distance metric for harmonicity of phoneme /n/, and another distance metric may be computed for shimmer of phoneme /m/. Additionally, or alternatively, distance metrics (or indication of change) may be determined for combinations of acoustic features over time period or instance.

In some embodiments, phoneme-features comparison logic 235 (or phoneme features comparer 274) includes computer instructions to generate or utilize a feature baseline for the user. A baseline may represent a healthy state, an illness state (e.g., influenza state or respiratory-infection state), a recovery state, or any other state of the user. Examples of other states may include the state of a user at a time instance or time interval (e.g., 30 days ago); the state of the user associated with an event (e.g., prior to a surgery or injury); the state of a user according to a condition (e.g., the state of the user from a time when the user is taking a medication, or during the time when the user lived in a polluted city); or a state associated with other criteria. For example, the baseline for a healthy state may be determined utilizing one or a plurality of feature sets corresponding to one or a plurality of time intervals (e.g., days) when the user was healthy.

A baseline determined based on a plurality of feature sets, each corresponding to a different time interval, may be referred to herein as a multi-reference or multiday baseline. In some instances, a multi-reference baseline comprises a plurality or group of feature sets, each corresponding to different time intervals. Alternatively, a baseline that is multi-reference may comprise a single representative feature set that is based on multiple feature sets from multiple time intervals (e.g., comprising an average or composite of feature set values from different time periods or instances, such as described previously). In some embodiments, a baseline may include statistical or supplemental data or metadata regarding the features. For instance, a baseline may comprise a feature set (which may be representative of multiple time intervals) and statistical variance, or a standard deviation of feature values, where multiple feature sets are used (e.g., a multi-reference baseline). Supplemental data may comprise contextual information, which may be associated with the time interval(s) of feature set(s) used for determining the baseline. Metadata may comprise information about the feature set(s) used to determine the baseline, such as information about the respiratory condition of the user at the time interval (e.g., the user is healthy, sick, recovering, etc.), or other information about the baseline. In some embodiments, a set of baselines may be determined to perform different comparisons, based on various criteria, as described herein.

Comparison of the feature vector(s), generated from a collected voice sample, to a baseline for a particular state may indicate how a user's condition or state compares to a known condition or state. In exemplary embodiments, the baseline is determined for the particular user such that comparison against the baseline will indicate whether the user's condition or state has changed or not. Alternatively, or additionally, the baseline may be determined for an at-large population or from a cohort of similar users. In some embodiments, different types of baselines are used for different feature sets. For examples, some features may be compared to a user-specific baseline while other features may be compared to a standard baseline determined from data from a population of individuals. In some embodiments, a user may specify (e.g., via settings 249) a particular voice sample, date, or time interval for use in determining a baseline. For example, the user may specify a date or a range of days via GUI, such as by selecting days on a calendar, corresponding to a known state or condition of the user, and may further provide information about the known state or condition (e.g., “please select at least one earlier date that you were healthy”). Similarly, during a recording session to obtain a voice sample, the user may indicate that the voice sample should be used to determine a baseline and may provide a corresponding indication of the user's condition or state. For instance, a GUI checkbox may be presented during the recording session for using the sample as a baseline for a healthy (or sick or recovering) state.

In some embodiments, phoneme-features comparison logic 235 may include computer instructions for generating and utilizing a multiday or multi-reference baseline. The multiday baseline may be rolling or fixed, for example. In particular, by performing a comparison of recent feature vector against this baseline, phoneme features comparer 274 may determine information indicating that the user's respiratory condition has changed, and whether the user is sick or well. Details regarding the determination of the user's respiratory condition, based on a comparison performed by phoneme features comparer 274, are described in connection with respiratory condition inference engine 278. Similarly, phoneme-features comparison logic 235 may comprise instructions for performing a plurality of comparisons utilizing a recent phoneme feature vector and a set of earlier vectors (or a multi-reference baseline), and instructions for comparing the difference measurements against each other, so that it may be determined (e.g., by respiratory condition inference engine 278) that a user's respiratory condition has changed and also that the user is sick (or healthy) or that the user's condition is getting better or worse. Additional details of performing multiple comparisons including comparisons of the distance measurements are described in connection with respiratory condition inference engine 278.

In some embodiments, the baseline may be dynamically defined automatically as more information about the user is obtained. For example, as normal variability in a user's voice information changes over time, the user's baseline may also change to reflect the user's current normal variability. Some embodiments may utilize an adaptive baseline that may be determined from a recent feature set or a plurality of recent feature sets (corresponding to a plurality of time intervals (e.g., days)) and is updated as new feature sets fitting the baseline criteria (e.g., healthy, sick, recovering) are determined. For example, a plurality of feature sets utilized for the adaptive baseline may follow a first in first out (FIFO) data flow, so that feature sets from older times are no longer considered as new feature sets for the baseline are determined (e.g., from more recent days). In this way, small variations or slow changes and adaptations that may occur in a user's voice may be excluded, due to the adaptive baseline. In some embodiments that utilize an adaptive baseline, parameters for the baseline (e.g., the number of feature sets to be included or a time window for recent feature sets to be included) may be configured in application settings (e.g., settings 249). In some instances of embodiments where feature sets from multiple time intervals (e.g., days) are utilized for a baseline, more recently determined feature sets may be weighted to carry more significance so that the baseline is up-to-date. Alternatively, or additionally, older (i.e., “stale”) feature sets, which correspond to earlier time periods or instances, may be weighted to decay over time or contribute less to the baseline.

In some embodiments, the particular features within a user's baseline may be tailored for that particular user. In this way, different users may have a different combination of phoneme features within their respective baselines and, accordingly, different phoneme features may be determined and utilized in monitoring the respiratory condition of each user. For example, in a first user's healthy voice sample, a particular acoustic feature (either generally or for a particular phoneme) may naturally fluctuate such that the feature may not be useful for detecting a change in the user's respiratory condition, whereas that feature may be useful and included in a baseline for another user.

In some embodiments, a baseline for a user may be correlated to contextual information, such as weather, time of the day, and/or season (i.e., time of the year). For example, a baseline for a user may be created from samples recorded during periods of high humidity. This baseline may be compared to phoneme feature vectors created from samples recorded during a period of high humidity. Conversely, a different baseline may be compared to a phoneme feature vector that is created from samples obtained during a period of relatively low humidity. In this way, there may be multiple baselines determined for a given user and utilized in different contexts.

Further, in some embodiments, a baseline may not be determined for a specific user but, rather, a specific cohort, such as individuals sharing a set of common characteristics. In an exemplary embodiment, a baseline may be respiratory-condition specific in that it may be determined utilizing data from individuals known to have the same respiratory condition (e.g., influenza, rhinovirus, COVID-19, asthma, chronic obstructive pulmonary disease (COPD), etc.). In some embodiments where a baseline may be dynamically defined as more information about a user is obtained, an initial baseline may be provided that is based on phoneme feature data from a population at large or cohort similar to the user. Over time, as more phoneme feature sets for the user are determined, the baseline may be updated using the user's phoneme feature sets, thereby personalizing the baseline for that user.

Some embodiments of respiratory-condition tracker 270 may include self-reporting data evaluator 276, which may collect self-reporting information from a user that may be correlated or considered for user diagnostics (e.g., determining the user's present respiratory condition) and/or forecasting a future condition. Self-reporting data evaluator 276 may collect this information from self-reporting tools 284 and/or contextual information determiner 2616. The information may be user-provided data or user-derived data (e.g., from sensors indicating temperature, breathing rate, blood oxygen, etc.) about how the user is feeling or the user's present condition(s). In one embodiment, this information includes the user self-reporting perceived severity of various symptoms related to a respiratory condition. For instance, the information may include a user's severity scores for post-nasal discharge, nasal obstruction, runny nose, thick nasal discharge with mucus, cough, sore throat, and need to blow nose.

Self-reporting data evaluator 276 may utilize the input data to determine a symptom score indicating a severity of a respiratory condition or symptom. For example, self-reporting data evaluator 276 may output a composite symptom score (CSS) that may be computed by combining scores for multiple symptoms. The individual symptom scores may be summed or averaged to obtain a composite symptom score. For example, in one embodiment, a composite symptom score may be determined by summing symptom scores (ranging from 0-5) for seven respiratory condition-related symptoms, resulting in a composite symptom score ranging between 0 and 35. A higher symptom score may indicate more severe symptoms. In one embodiment, the symptoms may include post-nasal discharge, nasal obstruction, runny nose, thick nasal discharge with mucus, cough, sore throat, and need to blow nose. In some embodiments, separate symptom scores may be generated for all symptoms, such as congestion-related symptoms, and non-congestion related symptoms.

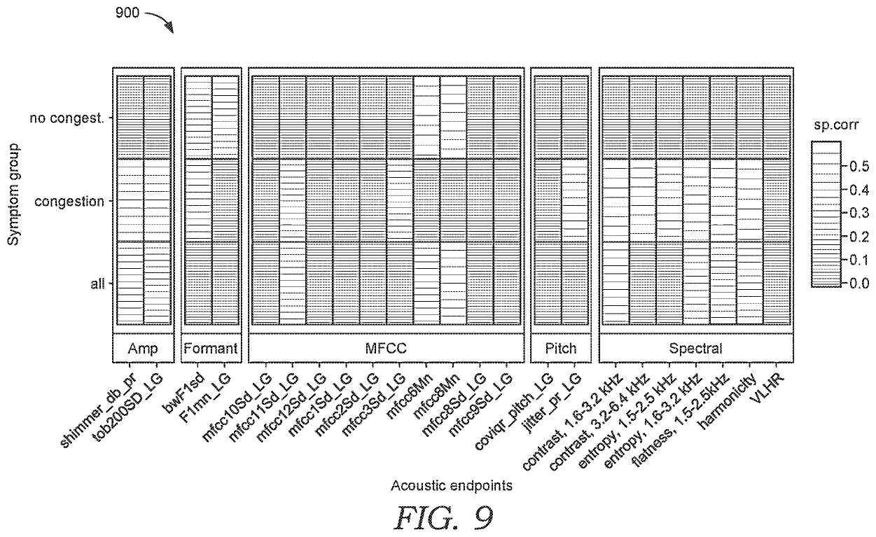

In some embodiments, self-reporting data evaluator 276 may associate a determined symptom score with phoneme feature(s) determined from a voice sample corresponding to a same time window as the user input that generated the score. In other embodiments, self-reporting data evaluator 276 may correlate a symptom score to a phoneme feature vector or a distance metric determined by comparing phoneme feature vectors. Symptom scores, such as a composite symptom score for all symptoms, including congestion-related symptoms or non-congestion-related symptoms, may be correlated to phoneme features by fitting an exponential decay model and correlating an acoustic feature value with a decay rate. The decay model may be utilized to estimate the magnitude and rate of change of symptoms. In one embodiment, score ˜ae^{-b (day-1)}+ϵ is utilized for the exponential decay model, where a represents the magnitude of change and b represents the decay rate. The exponential decay model may be implemented using non-linear mixed effect models with subject as a random effect from package nlme (version 3.1.144) of the R system (the R-project for Statistical Computing, which is accessible through the Comprehensive R Archive Network (CRAN)). Examples of correlations between phoneme feature vectors and symptom scores and between the phoneme feature vectors and or derived distance metrics are depicted in FIGS. 9 and 11A-B, respectively. The symptom score(s) generated by self-reporting data evaluator 276 and, in some embodiments, associations and/or correlations with phoneme feature vectors or distance measures may be stored in the user's individual record 240.

In some embodiments, self-reporting is initiated based on a detected change (e.g., user's condition is getting worse) or is initiated when a user is already sick. Initiation of self-reporting may also be based on user settings preferences, such as settings 249 in individual record 240. In some embodiments, self-reporting is initiated based on respiratory conditions detected from a user's collected voice samples. For example, self-reporting data evaluator 276 may determine to prompt a user to obtain self-reported symptom information based on a detection of the user's condition from voice analysis, which may be determined based on the comparison of feature vectors performed by phoneme features comparer 274.

Further, respiratory condition inference engine 278 may generally be responsible to determine or infer a user's current respiratory condition and/or predicting the user's future respiratory condition. This determination may be based on a user's acoustic features including changes detected in the feature values. As such, respiratory condition inference engine 278 may receive information about a user's phoneme features and/or the detected changes in features, which may be determined as a distance metric. Some embodiments of respiratory condition inference engine 278 may further utilize contextual information, which may be determined by contextual information determiner 2616, and/or user's self-reported data or an analysis of the self-reported data, such as a composite symptom score determined by self-reporting data evaluator 276. In one embodiment, the maximum phonation time, or the duration that a user sustains one or more particular phonemes, such as /a/, another cardinal vowel phonation, or other phonation may be used by respiratory condition inference engine 278 as an indicator of the user's respiratory condition. For example, a short maximum phonation time may indicate shortness of breath and/or decreased lung capacity, which may be associated with a worsening respiratory condition. Further, respiratory condition inference engine 278 may compare the acoustic features to one or more baselines to determine the user's respiratory condition. For example, a user's maximum phonation time may be compared to a user's baseline maximum phonation time to determine if the user's respiratory capacity is increasing or decreasing, where a decreasing maximum phonation time may indicate a worsening respiratory condition. Similarly, a decrease in the percentage of voiced frames in phonemes extracted from a voice sample of pre-determined duration may indicate a worsening respiratory condition. For a passage-reading voice sample, by way of examine and without limitation the following features may indicate a worsening respiratory condition: a decrease in speaking rate, an increase in average pause length, an increase in pause count, and/or a decrease in global SNR. Determining any of these changes may be done by comparing, such as described herein, a recent sample to a baseline, such as a user-specific baseline.

Respiratory condition inference engine 278 may utilize this input information to generate one or more respiratory-condition scores or classifications representing the user's current respiratory condition and/or future condition (i.e., a prediction). The output from respiratory condition inference engine 278 may be stored in results/inferred conditions 246 of a user's individual record 240, and may be presented to the user, as described in connection with an example GUI 5300 of FIG. 5C.

In some embodiments, respiratory condition inference engine 278 may determine a respiratory-condition score, which corresponds to the quantified changes detected in user's respiratory condition. Alternatively, or in addition, the respiratory-condition score or an inference of a user's respiratory-infection condition may be based on detected values of one or more specific phoneme features (i.e., a single reading, rather than a change), or based on a combination of one or more specific feature values, detected changes in feature values, and different rates of changes. In one embodiment, a respiratory-condition score may indicate a likelihood or probability that user has (or does not have) a respiratory condition (e.g., either generally for any condition or for a particular respiratory infection). For example, the respiratory-condition score may indicate that the user has a 60% likelihood of having a respiratory infection. In some aspects, the respiratory-condition score may comprise a composite score or a set of scores (e.g., a set of probabilities of the user having a set of respiratory conditions). For example, respiratory condition inference engine 278 may generate a vector of specific respiratory conditions with corresponding likelihoods that the user has each of the conditions, such as, allergies, 0.2; rhinovirus, 0.3; COVID-19, 0.04; and so on. Alternatively, or in addition, the respiratory-condition score may indicate a difference of the user's current condition from a known healthy condition or may be based on a comparison of the user's current condition to a baseline or healthy condition of the user, such as described herein.

In many instances, respiratory condition inference engine 278 may determine (or the respiratory-condition score may indicate) a change or difference from the user's healthy state (or a probability of respiratory infection), when the user does not feel symptomatic. This capability is an advantage and improvement over conventional technologies that rely on subjective data. On the other hand, the embodiments of the technologies provided herein may detect the onset of a respiratory infection before a user feels symptomatic, rather than relying on subjective data. These embodiments may be particularly useful for combatting respiratory-based pandemics, such as SARS-CoV-2 (COVID-19), by providing an earlier warning of respiratory infection than conventional approaches. For example, the respiratory-condition score (or a determination about a user's respiratory condition by respiratory condition inference engine 278) indicating a possible infection may inform a user to self-quarantine, social distance, wear a facemask, or take other precautions sooner than the user might otherwise.

In some embodiments, the respiratory-condition score, which may indicate or correspond to a probability of the user having a respiratory infection, may be represented as a value relative to a user's healthy state. For example, a respiratory-condition score of 90 out of 100 (with 100 representing a healthy state) may indicate that detected change(s) of the user's respiratory condition are 90% of the user's normal or healthy state (i.e., a 10% change). In this example, the user may feel healthy with a respiratory-condition score of 90, but the score may indicate that the user is developing (or still recovering from) a respiratory infection. Similarly, a respiratory-condition score of 20 may indicate that a user is probably sick (i.e., the user likely has a respiratory infection), while a respiratory-condition score of 40 may also indicate the user is probably sick but less likely to be as sick (or may not be as sick) as indicated by a respiratory-condition score of 20. For example, where a respiratory-condition score corresponds to a probability, then the respiratory-condition score of 20 may indicate that the user has a higher probability of having an infection than the respiratory-condition score of 40. But where the respiratory-condition score reflects a difference between the user's current state and a healthy baseline, then the respiratory-condition score of 40 may correspond to a smaller detected change from the baseline than the respiratory-condition score of 20 and, thus, may indicate the user may not be as sick. In some instances, a user's respiratory-condition score may be indicated using a color or a symbol, rather than or in addition to a number. For example, green may indicate that the user is healthy, while yellow, orange, and red may represent increasing differences from the user's healthy state, which may indicate increasing likelihoods that the user has a respiratory infection. Similarly, emoticons (e.g., smiley vs. frowny or sick faces) may be utilized to represent respiratory-condition scores.

It should be understood that embodiments herein may be used to characterize a state of respiratory infection for a user based on phoneme feature information (including changes in phoneme features) and, in some embodiments, based further on contextual information (such as measured physiological data) and/or self-reported symptom scores from the user. Accordingly, in some instances, severe respiratory infection and a mild respiratory infection both may manifest the same phoneme features (or changes in features). Thus, in these instances, different respiratory-condition scores may not be useful for indicating that a user is “more sick” or “less sick,” but instead may indicate just that the user has (or does not have) a respiratory infection (i.e., a binary indication) or indicate a probability that the user is sick, or may represent a difference from the user's current state versus a healthy state, which may indicate a sign of a respiratory infection.

Furthermore, monitoring changes in respiratory-condition scores when correlated to a user's treatment for a respiratory infection (which may be received as contextual information), such as taking a prescription medication, may indicate efficacy of the treatment. For example, a user who is diagnosed with a respiratory infection is prescribed an antibiotic by their clinician and instructed to use a respiratory infection monitor app on their smartphone, such as a respiratory-infection monitor app 5101 described in connection with FIG. 5A. An initial respiratory-condition score (or a first set of respiratory-condition scores) may be determined from user voice samples collected as described herein. After some time interval, such as a week, a second respiratory-condition score may indicate a change in the user's respiratory condition. A change indicating the user's condition is improving (which may be determined as described below) may imply that the antibiotic is working. A change indicating that the user's condition is not improving or is staying the same may imply that the antibiotic is not working, in which case the user's clinician may want to prescribe a different treatment. In this way, embodiments of the technologies described herein may determine an objective, such as quantifiable information about changes to the user's respiratory conditions, antibiotics prescribed for treatment of respiratory infections may be utilized more carefully and deliberately, thereby prolonging their efficacy and minimizing antimicrobial resistance.

In some embodiments, respiratory condition inference engine 278 may utilize user-condition inference logic 237 to determine a respiratory-condition score or to make inferences and/or predictions regarding a user's respiratory condition. User-condition inference logic 237 may include rules, conditions, associations, machine learning models, or other criteria for inferring and/or predicting a likely respiratory condition from voice-related data. User-condition inference logic 237 may take different forms depending on the mechanism(s) used and intended output. In one embodiment, user-condition inference logic 237 may include one or more classifier models to determine or infer a user's current (or recent) respiratory condition and/or one or more predictor models to forecast a user's likely future respiratory condition. Examples of classifier models may include, without limitation, decision tree(s) or random forests, Naive Bayes, neural network(s), pattern recognition models, other machine-learning models, other statistical classifiers, or combinations (e.g., ensemble). In some embodiments, user-condition inference logic 237 may include logic for performing clustering or unsupervised classification techniques. Examples of prediction models may include, without limitation, regression techniques (e.g., linear or logistic regression, least squares, generalized linear model (GLM), multivariate adaptive regression splines (MARS), or other regression processes), neural network(s), decision tree(s) or random forest, or other predictive models or combinations (e.g., ensemble) of models.

As described above, some embodiments of respiratory-condition inference engine 278 may determine a probability of the user having or developing a respiratory infection. In some instances, the probability may be based on the user's acoustic features, including changes detected in the features and the output of a classifier or prediction model, or rules or conditions being satisfied. For example, according to an embodiment, user-condition inference logic 237 may include rules for determining a probability of a respiratory infection based on changes to phoneme feature values satisfying a particular threshold (e.g., a condition-change threshold, as described herein) or based on a degree of detected change(s) occurring to one or multiple phoneme feature values. In one embodiment, user-condition inference logic 237 may include rules for interpreting a detected change or difference between a user's current respiratory condition and a baseline to determine a likelihood that the user has a respiratory infection. In a further embodiment, multiple recent evaluations of a user's respiratory condition (i.e., multiple comparisons from recent times to earlier times) may contribute to a probability. By way of example, and without limitation, if the user shows a change in respiratory condition two days in a row, then a higher probability of respiratory infection may be provided than a user showing the change after only a single day. In one embodiment, the detected changes and/or rates of change may be compared to a set of one or more patterns of known phoneme-feature changes for particular respiratory infections or a set of thresholds applied to feature changes and corresponding to known respiratory infections, and a likelihood of infection determined based on the comparison. Further, in some embodiments, user-condition inference logic 237 may utilize contextual information, such as physiological information or information about regional outbreaks of respiratory-infectious diseases, to determine a probability of the user having the respiratory infection.

User-condition inference logic 237 may comprise computer instructions and rules or conditions for performing a comparison of a determined change of the acoustic feature information (e.g., a change in feature set values, feature vector distance measurements and other data), or a determined rate of change of the acoustic feature information against one or more thresholds, which may be referred to herein as condition-change thresholds. For example, a distance measurement of two feature vectors, corresponding to recent and earlier time intervals, respectively, may be compared to a condition-change threshold. The condition-change threshold may be utilized as a detector (e.g., as an outlier detector), such that based on the comparison, if the threshold is satisfied (e.g., exceeded), then the change in the user's respiratory condition is considered as detected. The condition-change threshold may be determined so that a meaningful change in the user's condition may be detected, but minor variations, which are insignificant but that nevertheless changes, are not detected as (or determined to be) changes to the user's respiratory condition. For instance, some embodiments that utilize a multiday baseline may employ a condition-change threshold determined to be two standard deviations of the multiday baseline feature values, as further described herein.

In some embodiments, a condition-change threshold is specific to a state of the user's condition (e.g., infected or not infected), and if a magnitude of change between feature vectors satisfies a condition-change threshold, it may be determined that the user's condition has changed. The threshold(s) may also be used to determine a trend in the respiratory condition generally as well as to determine the likely presence of a respiratory condition. In one embodiment, if a comparison (which may be performed by phoneme features comparer 274) satisfies (e.g., exceeds) a condition-change threshold, it may be determined that the user's respiratory condition is changing by a certain magnitude (as specified by the condition-change threshold), and thus the user's condition is improving or worsening (i.e., a trend). In this way, minor changes that do not satisfy the condition-change threshold, in this embodiment, may not be considered or may indicate that the user's condition is effectively unchanged.

In some embodiments, a condition-change threshold may be weighted, applied to only a portion of the phoneme features, and/or may comprise a set of thresholds for characterizing changes in each phoneme feature of a feature vector (or phoneme feature set), or for a subset of the features. For example, a small change in a first phoneme feature may be significant, while a small change in a second phoneme feature may not be as significant or may even be commonly occurring. Thus, it may be helpful to know that the first feature value has changed, even if a little, and also helpful to know that the second feature value has changed to a greater degree. Accordingly, a smaller first condition-change threshold (or a weighted threshold) may be used for this first phoneme feature so that even small changes may satisfy this first condition-change threshold, and a higher (second) condition-change threshold (or a threshold with a different weighting) may be used for the second phoneme feature. Such a weighted or varied condition-change threshold application may be utilized to detect or monitor certain respiratory infections where a particular phoneme feature is determined to be more sensitive (i.e., changes of this phoneme feature are more indicative of a change to the user's respiratory condition).

In some embodiments, the condition-change threshold is based on a standard deviation of a baseline that is used for the comparison against recent acoustic feature values for the user. For example, a baseline, such as a multiday baseline, may be determined (e.g., by phoneme-features comparison logic 235) to include feature information for a plurality of time intervals from when the user was healthy (or sick), for example. A standard deviation may be determined based on the feature values of the features from different time intervals (e.g., days) used in the baseline. The condition-change threshold may be determined based on the standard deviation (e.g., a threshold of two standard deviations is utilized). For example, a user may be determined to have a respiratory infection or other condition if a comparison of a recent phoneme feature set versus a healthy baseline (or similar detected change in the user's phoneme feature values over time period or instance) satisfies two standard deviations from the baseline. In this way, the comparison is more robust. By way of example, and without limitation, minor variations in a user's acoustic features that might occur from day-to-day when the user is healthy are factored into the condition-change threshold(s). In some instances, multiple thresholds may be utilized, based on standard deviations, in order to determine or quantify a degree of the difference between the user's current respiratory condition and the baseline. For example, in one embodiment, a user may be determined to have a low probability of a respiratory infection if the comparison to a healthy baseline (or similar detected change in the user's phoneme feature values over time) satisfies two standard deviations from the baseline, and that the user may be determined to have a high probability of a respiratory infection if the comparison satisfies three standard deviations from the baseline.

In some embodiments, the condition-change threshold determined according to user-condition inference logic 237 may be modified (e.g., by the user, a clinician, or a caregiver of the user) or may be pre-determined (e.g., by a clinician, a caregiver or an application developer). The condition-change threshold may also be based on reference population data or determined for the particular user. For instance, the condition-change threshold may be set based on user's specific health information (e.g., health diagnosis, medications, or health record data) and/or personal information (e.g., age, user behavior or activity such as singing or smoking). In addition, or alternatively, a user (or a caregiver) may set or adjust the condition change threshold as a setting, such as in settings 249 of individual record 240. In some aspects, the condition-change threshold may be based on a particular respiratory infection that is being monitored or detected. For example, user-condition inference logic 237 may include logic for utilizing a different threshold (or a set of thresholds) for monitoring different possible respiratory infections or conditions. Accordingly, a particular threshold may be utilized when the user's condition is known (e.g., following a diagnosis) or suspected, which may be determined, in some instances, from contextual information or self-reported symptom information. In some embodiments, more than one condition-change threshold may be applied.

In some embodiments, user-condition inference logic 237 may comprise computer instructions for performing outlier (or anomaly) detection and may take the form of an outlier detector (or utilize an outlier-detection model) to detect a likely incidence of respiratory infection to the user. For example, in one embodiment, the user-condition inference logic 237 may include a set of rules to determine and utilize a standard deviation of a baseline feature set (e.g., a multiday baseline) as a threshold for outlier detection, as further described herein. In other embodiments, user-condition inference logic 237 may take the form of one or more machine-learning models utilizing an outlier detection algorithm. For instance, user-condition inference logic 237 may include one or more probabilistic models, linear regression models, or proximity-based models. In some aspects, such models may be trained on the user's data so that the models detect user-specific variability. In other embodiments, models may be trained to utilize reference information for respiratory-condition specific cohort. For example, a model for detecting a particular respiratory condition, such as influenza, asthma, and chronic obstructive pulmonary disease (COPD), are trained with data for individuals known to have such a condition. In this way, user-condition inference logic 237 may be specific to a type of respiratory condition being monitored, determined, or forecasted.

In some embodiments, the output of respiratory condition inference engine 278, utilizing user-condition inference logic 237, is a prediction or forecast. The prediction may be determined based on changes, rates of changes, and/or patterns of changes detected in phoneme features or respiratory-condition scores, and may utilize trend analysis, regression, or other prediction model described herein. In some embodiments, the prediction may include a corresponding prediction probability and/or a future time interval for the prediction (e.g., the user has a 70% likelihood of developing a respiratory infection by next week). One embodiment predicts when a user is likely to be healthy again based on a detected rate of change in the user's phoneme features showing a trend of improvement of the user's respiratory condition (see, e.g., FIG. 4E for an example depicting this embodiment). In some instances, a prediction may be provided in the form of a trend or outlook for the user (e.g., the user is recovering or worsening) or may be provided as a probability/likelihood that the user will get sick or recover. Some embodiments may compare patterns of changes to a user's phoneme features or respiratory-condition scores to determine patterns from a reference population of people (e.g., a population at large or a population similar to the user, such as a cohort having a similar respiratory condition), in order to determine a likely future forecast for the user's respiratory condition. In some embodiments, respiratory condition inference engine 278 or user-condition inference logic 237 may include functionality for assembling one or more patterns of user phoneme feature vectors. The patterns may be correlated with self-reporting input or with symptom scores or determinations generated from self-reporting input, such as composite symptom scores. The user phoneme feature patterns may then be analyzed to predict a future respiratory condition for the particular user. Alternatively, user patterns from other users, either a reference population representing the population at large, a population of individuals having a particular respiratory condition (e.g., a cohort having influenza, asthma, rhinovirus, chronic obstructive pulmonary disease (COPD), COVID-19, etc.) or a population of individuals similar to the user, may be utilized for forecasting a future respiratory condition of the particular user. Example illustrations showing predictions of respiratory conditions are provided in FIGS. 4E (element 447) and 5C (element 5316).

User-condition inference logic 237 may consider patterns or rates of changes in phoneme feature vectors, in some embodiments, and/or may consider geo-localized information, such as infection outbreaks in the area in which the user is present. For example, a certain pattern (or rate(s)) of change of all or certain phoneme features may be indicative of particular respiratory infections, such as those that manifest a progression of respiratory conditions or symptoms (e.g., congestion for several days typically followed by sore throat, typically followed by laryngitis).

In some embodiments, user-condition inference logic 237 may include computer instructions for determining and/or comparing multiple change(s) or rate(s) of change(s) of the phoneme feature information. For example, a first comparison (or a set of comparisons) between a recent phoneme feature vector and a first earlier phoneme feature vector may indicate that a user's respiratory condition has changed. In an embodiment, whether that change indicates the user's condition is improving or worsening may be determined by performing additional comparisons. For example, a second comparison of the recent phoneme feature vector to a healthy baseline feature vector or a second earlier phoneme feature vector from a time period or instance when the user is known to be healthy may be determined. Further, a third comparison between the first earlier phoneme feature vector and baseline or second earlier phoneme feature vector may be determined. The change(s) detected between the second comparison and third comparison may be compared (in a fourth comparison) to determine whether the user's respiratory condition is improving (e.g., where the difference between the recent phoneme feature vector vs. the healthy baseline is less than the difference between the first earlier phoneme feature vector and the healthy baseline) or worsening (e.g., where the difference between the recent phoneme feature vector vs. the healthy baseline is greater than the difference between the first earlier phoneme feature vector and the healthy baseline). Further, additional comparisons to a threshold indicating a degree of change may be utilized to determine a degree to which user's respiratory condition has worsened or improved, how close to recovery is the user (e.g., where phoneme feature values are returning to or near those of the healthy baseline), or when the user may expect to be at a recovery state (e.g., based on a rate or change(s) in the user's condition in a trend showing improvement).

In some embodiments, user-condition inference logic 237 may include one or more decision trees (or random forest or other model) for incorporating a user's self-reporting and/or contextual data, which may include physiological data, such as user sleep information (if available), information about recent user activity, or user location information, in some instances. For example, if a user's voice-related data indicates the voice is hoarse and it is determined, from contextual information, that the user's location was at an arena venue the previous night and had a calendar entry titled “playoff tournament” for the previous night, user-condition inference logic 237 may determine that it is more likely that observed changes in the user's voice data are a result of the user attending a sporting event rather than a respiratory infection.

In some embodiments, user-condition inference logic 237 may include computer instructions for determining a likely risk of the user transmitting a detected respiratory-related infectious agent. For example, a transmission risk may be determined based on rules or conditions applied to a respiratory condition or likely future condition determined by respiratory condition inference engine 278, or a clinician's diagnosis of the user having respiratory infection. The transmission risk may be binary (e.g., the user likely is/is not contagious), categorical (e.g., a low, medium, or high risk of transmission), or may be determined as a probability or transmission risk score, which may indicate the likelihood of transmissibility. In some instances, the transmission risk may be based on a particular respiratory infection the user has or likely has (e.g., influenza, rhinovirus, COVID-19, certain types of pneumonia, etc.). As such, a rule may specify that a user having a particular condition (e.g., COVID-19) is contagious for a set duration of time, which may be fixed or vary based on the user's condition. For example, the rule may specify that the user is contagious for 24 hours after a determination by respiratory condition inference engine 278 that the user is likely no longer experiencing respiratory infection. Moreover, a transmission risk may be static for the entire duration of the user experiencing (or likely experiencing) respiratory infection or may vary based on the user's state or progression of respiratory infection. For instance, a transmission risk may vary based on a detected change, trend, pattern, rate of change, or analysis of detected changes of the user's respiratory condition (or voice-related data) over a recent time interval (e.g., over the past week or from a time when the user is first determined by respiratory condition inference engine 278 to possibly have respiratory infection). The transmission risk may be provided to the user or utilized (e.g., by respiratory condition inference engine 278, another component of system 200, or a clinician) to determine recommendations for the user, such as avoiding close contact with others or wearing a facemask. One example of a transmission risk determined in accordance with an embodiment of user-condition inference logic 237 by respiratory condition inference engine 278 is depicted in element 5314 of FIG. 5C.

In some embodiments, user-condition inference logic 237 may include rules, conditions, or instructions for determining and/or providing a recommendation corresponding to a respiratory condition, forecast, transmission risk, or other determination by respiratory condition inference engine 278. The recommendation may be provided to an end user such as a patient, a caregiver, or a clinician associated with the user (e.g., decision support recommendation). For example, the recommendation determined for the user or caregiver may comprise one or more recommended practices to minimize transmission, manage a respiratory infection, or minimize a likelihood of the infection to worsen. In some embodiments, user-condition inference logic 237 may comprise computer instructions for accessing a database of health information, which may be associated with a determined respiratory infection or other determination by respiratory condition inference engine 278 and providing at least a portion of the information to a user, a caregiver, or a clinician. Additionally, or alternatively, the recommendations may be determined utilizing (or selected or assembled from) information in a health information database.

In some embodiments, recommendations may be tailored to the user based on the user's current and/or historical information (e.g., historical voice-related data, previously determined respiratory conditions, trends or changes in the user's respiratory condition, or the like), and/or contextual information, such as symptoms, physiological data, or geographical location. For example, in one embodiment, the information about the user may be utilized as selection or filtering criteria to identify relevant information in a database of health information for use in determining a recommendation tailored to the user.

A recommendation may be provided to user, caregiver, or clinician, and/or stored in individual record 240 associated with the user, such as in results/inferred conditions 246. In some embodiments that access the health information database, the database may be stored on storage 250 and/or on a remote server or in the cloud environment. An example of a recommendation determined in accordance with an embodiment of user-condition inference logic 237 by respiratory condition inference engine 278 is depicted in element 5315 of FIG. 5C.

As shown in FIG. 2, example system 200 also includes a decision support tool(s) 290, which may comprise various computing applications or services for consuming output determinations of components of system 200, such as the user respiratory conditions or predictions determined by respiratory-condition tracker 270 (or one of its subcomponents, such as respiratory condition inference engine 278) or from storage (e.g., from results/inferred conditions 246 in a user's individual record 240). Decision support tool(s) 290 may utilize this information to enable therapeutic and/or preventative actions, in accordance with some embodiments. In this way, decision support tool(s) 290 may be utilized by a monitored user and/or a caregiver of the monitored user. This decision support tool(s) 290 may take the form of a standalone application on a client device, a web application, a distributed application or service, and/or a service on an existing computing application. In some embodiments, one or more decision support tool(s) 290 are part of respiratory-infection monitoring or tracking application, such as respiratory-infection monitor app 5101 described in connection with FIG. 5A.

One exemplary decision support tool includes a sick monitor 292. Sick monitor 292 may comprise an app operating on the user's smartphone (or smart speaker or other user device). The sick monitor 292 app may monitor a user's speech and inform the user and/or the user's care provider whether or not the user is getting sick or recovering from a respiratory infection, such as rhinovirus or influenza. In some embodiments, sick monitor 292 may request permission to listen to a user to collect voice-related data or, in some aspects, other data. Sick monitor 292 may generate a notification or an alert to the user indicating whether or not the user is getting sick, is likely sick, or recovering. In some embodiments, sick monitor 292 may initiate and/or schedule a treatment recommendation based on the respiratory condition determination and/or prediction. The notification or alert may include a recommended action for an intervening action, such as treatment, based on the respiratory condition determination and/or prediction. A treatment recommendation may comprise, by way of example and without limitation, recommended actions for the user to take (e.g., wear a facemask), an over-the-counter medicine, consultation with a clinician, and/or testing that is recommended to confirm the presence of a respiratory infection and/or to treat the respiratory infection and/or the resulting symptoms. For example, sick monitor 292 may recommend that the user schedule a visit with a healthcare provider and/or get tested for confirmation of a respiratory condition. In some embodiments, sick monitor 292 may initiate or facilitate scheduling of the doctor's appointment and/or testing appointment. Alternatively, or additionally, sick monitor 292 may recommend or order treatment, such as over-the-counter medicine.

Embodiments of sick monitor 292 may recommend that the user inform other individuals within the user's home to take precautions, such as maintaining a minimum distance, to prevent the infection from spreading. In some embodiments, sick monitor 292 may recommend this notification and, upon the user affirmatively authorizing this notification, sick monitor 292 may initiate notifications to user devices associated with other users in the infected user's home. Sick monitor 292 may identify the relevant user devices from information stored in the user's individual record 240, such as from user account(s)/device(s) 248. In some embodiments, sick monitor 292 may correlate other sensed data (e.g., physiological data such as heart rate, temperature, sleep, and the like), other contextual data, such as information about respiratory infection outbreaks in the user's region, or data input from the user (such as symptom information provided via self-reporting tools 284) with the determination and/or prediction of a respiratory condition to make a recommendation.

In one embodiment, sick monitor 292 may be part of, or operate in conjunction with, an infection contact tracing application. In this way, the information about early detection of possible respiratory infection for a first user may be communicated automatically to other individuals that the first user contacted. Additionally, or alternatively, the information may be used to initiate respiratory-infection monitoring of those other individuals. For example, the other individuals may be notified of a possible contact with an infected person and prompted to download and use sick monitor 292 or a respiratory-infection monitoring application, such as respiratory-infection monitoring app 5101 described in connection with FIG. 5A. In this way, other individuals may be notified and begin monitoring even before the first user feels sick (i.e., before the first user is symptomatic).

Another example decision support tool(s) 290 is a prescription monitor 294, as shown in FIG. 2. Prescription monitor 294 may utilize determinations and/or predictions about user's respiratory condition, such as whether the user has respiratory infection or not, to determine whether a prescription should be refilled or not. Prescription monitor 294 may determine, from user's individual record 240, for example, whether the user has a current prescription for the detected or forecasted respiratory condition or not. Prescription monitor 294 may also determine the prescription directions for a frequency of taking the medication, a last fill date of the medication, and/or how many refills are available. Prescription monitor 294 may determine whether a refill of the prescription is needed or not based on a determination that the user has a present respiratory infection or a prediction that the user will have one or will show symptoms in the near future.

Some embodiments of prescription monitor 294 may also determine whether the user is taking a medicine, either by sensed data or user's input via self-reporting tools 284, or not. Information indicating whether or not the user is taking the prescribed medicine is used by prescription monitor 294 to determine if or when a current prescription may fall short. Prescription monitor 294 may issue an alert or notification indicating to the user that a prescription be refilled. In one embodiment, prescription monitor 294 issues a notification recommending refill of a prescription, after the user takes affirmative steps to request a refill. Prescription monitor 294 may initiate ordering the refill through a pharmacy, whose information may be stored in the user's individual record 240 or input by the user at the time of the refill. Aspects of an example prescription monitoring service, such as prescription monitor 294, are depicted in FIG. 4F.

Another example decision support tool(s) 290 is a medication efficacy tracker 296, as shown in FIG. 2. Medication efficacy tracker 296 may utilize determinations and/or predictions about a user's respiratory condition, such as whether the user's condition is improving or worsening, to determine whether the effectiveness of a medication being taken by the user is effective or not. As such, medication efficacy tracker 296 may determine, from user's individual record 240, whether the user has a current prescription or not. Medication efficacy tracker 296 may determine whether the user is actually taking the medicine, either by sensed data or the user's input via self-reporting tools 284, or not. Medication efficacy tracker 296 may also determine the prescription directions and may determine whether the user is taking the medication in accordance with the prescribed directions or not.

In some embodiments, medication efficacy tracker 296 may correlate the inferences or forecasts about a respiratory condition based on utilizing voice-related data to determine whether the user is taking medication or not and to further determine whether the medication is effective or not. For example, if the user is taking medicine as prescribed and the respiratory condition is worsening or not improving, it may be determined that the prescription medication is not effective in this instance for the particular user. As such, medication efficacy tracker 296 may recommend that the user consult a clinician to change the prescription or may automatically communicate an electronic notification to the user's doctor or a clinician so that the clinician may consider modifying the prescribed treatment.

In some embodiments, medication efficacy tracker 296 additionally, or alternatively, operates on or in conjunction with a device of a clinician of the monitored user, such as clinician user device 108 of FIG. 1. For example, a clinician may prescribe a sick patient with a medication, such as an antibiotic, for a respiratory infection and may, in conjunction, prescribe the patient a medication efficacy tracking application (such as 296) to monitor the patient's voice-related data in accordance with embodiments of this disclosure. Upon determining that the user is worsening or not improving, medication efficacy tracker 296 may notify the clinician of the inferences or forecasts of the patient's respiratory condition. In some instances, medication efficacy tracker 296 may further make recommendations to change the prescribed treatment for the patient.

In another embodiment, medication efficacy tracker 296 may be utilized as a part of a study or trial for medication and may analyze determinations and/or forecasts of respiratory conditions for multiple participants to determine whether or not the studied medication is effective for the group of participants. Additionally or alternatively, in some embodiments, medication efficacy tracker 296 may be utilized as part of a study or trial in conjunction with a sensor (e.g., sensor(s) 103) and/or self-reporting tools 284 to determine whether there are side effects of the medication, such as respiratory-related side-effects (such as, for example, cough, congestion, runny nose) or non-respiratory-related side effects (such as, for example, fever, nausea, inflammation, swelling, itching).

Some embodiments of decision support tools 290 described above include aspects for treating a user's respiratory condition. Treatment may be targeted to reduce the severity of the respiratory condition. Treating the respiratory condition may include determining a new treatment protocol, which may include a new therapeutic agent(s), a dosage of a new agent or a new dosage of an existing agent being taken by the user or a dosage of a new agent, and/or a manner of administering a new agent or a new manner of administration of an existing agent taken by the user. A recommendation for the new treatment protocol may be provided to the user or caregiver for the user. In some embodiments, a prescription may be sent to the user, the user's caregiver, or a user's pharmacy. In some instances, treatment may include refilling an existing prescription without making changes. Further embodiments may include administering the recommended therapeutic agent(s) to the user in accordance with the recommendation treatment protocol and/or tracking the application or use of the recommended therapeutic agent(s). In this way, embodiments of the disclosure may better enable controlling, monitoring, and/or managing the use or application of therapeutic agents for treating a respiratory condition, which would not only be beneficial on a user's condition but could help healthcare providers and drug manufacturers, as well as others within the supply chain, better comply with regulations and recommendations set by the Food and Drug Administration and other governing bodies.

In example aspects, treatment includes one or more therapeutic agents from the following:
PLpro inhibitors, Apilomod, EIDD-2801, Ribavirin, Valganciclovir, β-Thymidine, Aspartame, Oxprenolol, Doxycycline, Acetophenazine, Iopromide, Riboflavin, Reproterol, 2,2′-Cyclocytidine, Chloramphenicol, Chlorphenesin carbamate, Levodropropizine, Cefamandole, Floxuridine, Tigecycline, Pemetrexed, L(+)-Ascorbic acid, Glutathione, Hesperetin, Ademetionine, Masoprocol, Isotretinoin, Dantrolene, Sulfasalazine Anti-bacterial, Silybin, Nicardipine, Sildenafil, Platycodin, Chrysin, Neohesperidin, Baicalin, Sugetriol-3,9-diacetate, (—)-Epigallocatechin gallate, Phaitanthrin D, 2-(3,4-Dihydroxyphenyl)-2-[[2-(3,4-dihydroxyphenyl)-3,4-dihydro-5,7-dihydroxy-2H-1-benzopyran-3-yl]oxy]-3,4-dihydro-2H-1-benzopyran-3,4,5,7-tetrol, 2,2-di(3-indolyl)-3-indolone, (S)-(1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl-2-amino-3-phenylpropanoate, Piceatannol, Rosmarinic acid, and/or Magnolol;
3CLpro inhibitors, Lymecycline, Chlorhexidine, Alfuzosin, Cilastatin, Famotidine, Almitrine, Progabide, Nepafenac, Carvedilol, Amprenavir, Tigecycline, Montelukast, Carminic acid, Mimosine, Flavin, Lutein, Cefpiramide, Phenethicillin, Candoxatril, Nicardipine, Estradiol valerate, Pioglitazone, Conivaptan, Telmisartan, Doxycycline, Oxytetracycline, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl5-((R)-1,2-dithiolan-3-yl) pentanoate, Betulonal, Chrysin-7-O-β-glucuronide, Andrographiside, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl 2-nitrobenzoate, 2β-Hydroxy-3,4-seco-friedelolactone-27-oic acid (S)-(1S,2R,4aS,5R,8aS)-1-Formamido-1,4a-dimethyl-6-methylene-5-((E)-2-(2-oxo-2,5-dihydrofuran-3-yl)ethenyl) decahydronaphthalen-2-yl-2-amino-3-phenylpropanoate, Isodecortinol, Cerevisterol, Hesperidin, Neohesperidin, Andrograpanin, 2-((1R,5R,6R,8 aS)-6-Hydroxy-5-(hydroxymethyl)-5,8a-dimethyl-2-methylenedecahydronaphthalen-1-yl)ethyl benzoate, Cosmosiin, Cleistocaltone A, 2,2-Di(3-indolyl)-3-indolone, Biorobin, Gnidicin, Phyllaemblinol, Theaflavin Rosmarinic acid, Kouitchenside I, Oleanolic acid, Stigmast-5-en-3-ol, Deacetylcentapicrin, and/or Berchemol;
RdRp inhibitors, Valganciclovir, Chlorhexidine, Ceftibuten, Fenoterol, Fludarabine, Itraconazole, Cefuroxime, Atovaquone, Chenodeoxycholic acid, Cromolyn, Pancuronium bromide, Cortisone, Tibolone, Novobiocin, Silybin, Idarubicin Bromocriptine, Diphenoxylate, Benzylpenicilloyl G, Dabigatran etexilate, Betulonal, Gnidicin, 2β,30β-Dihydroxy-3,4-seco-friedelolactone-27-lactone, 14-Deoxy-11,12-didehydroandrographolide, Gniditrin, Theaflavin (R)-((1R,5aS,6R,9aS)-1,5a-Dimethyl-7-methylene-3-oxo-6-((E)-2-(2-oxo-2,5-dihydrofuran-3-yl)ethenyl)decahydro-1H-benzo[c]azepin-1-yl)methyl2-amino-3-phenylpropanoate, 2β-Hydroxy-3,4-seco-friedelolactone-27-oic acid, 2-(3,4-Dihydroxyphenyl)-2-[[2-(3,4-dihydroxyphenyl)-3,4-dihydro-5,7-dihydroxy-2H-1-benzopyran-3-yl]oxy]-3,4-dihydro-2H-1-benzopyran-3,4,5,7-tetrol, Phyllaemblicin B, 14-hydroxycyperotundone, Andrographiside, 2-((1R,5R,6R,8aS)-6-Hydroxy-5-(hydroxymethyl)-5,8a-dimethyl-2-methylenedecahydro naphthalen-1-yl)ethyl benzoate, Andrographolide, Sugetriol-3,9-diacetate, Baicalin, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl 5-((R)-1,2-dithiolan-3-yl)pentanoate, 1,7-Dihydroxy-3-methoxyxanthone, 1,2,6-Trimethoxy-8-1(6-O-β-D-xylopyranosyl-β-D-glucopyranosyl)oxyl-9H-xanthen-9-one, and/or 1,8-Dihydroxy-6-methoxy-2-[(6-O-β-D-xylopyranosyl-β-D-glucopyranosyl)oxy]-9H-xanthen-9-one, 8-(β-D-Glucopyranosyloxy)-1,3,5-trihydroxy-9H-xanthen-9-one.

In example aspects, treatment includes one or more therapeutic agents for treating a viral infection, such as SARS-CoV-2, which causes COVID-19. As such, the therapeutic agents may include one or more SARS-CoV-2 inhibitors. In some embodiments, treatment includes a combination of one or more SARS-CoV-2 inhibitors with one or more of the therapeutic agents listed above.

In some embodiments, treatment includes one or more therapeutic agents selected from any of the previously identified agents as well as the following:
Diosmin, Hesperidin, MK-3207, Venetoclax, Dihydroergocristine, Bolazine, R428, Ditercalinium, Etoposide, Teniposide, UK-432097, Irinotecan, Lumacaftor, Velpatasvir, Eluxadoline, Ledipasvir, Lopinavir /Ritonavir+Ribavirin, Alferon, and prednisone;
dexamethasone, azithromycin and remdesivir as well as boceprevir, umifenovir and favipiravir;
α-ketoamides compounds 11r, 13a and 13b, as described in Zhang, L.; Lin, D.; Sun, X.; Rox, K.; Hilgenfeld, R.; X-ray Structure of Main Protease of the Novel Coronavirus SARS-CoV-2 Enables Design of α-Ketoamide Inhibitors; bioRxiv preprint doi: https://doi.org/10.1101/2020.02.17.952879;
RIG 1 pathway activators, such as those described in U.S. Pat. No. 9,884,876;
protease inhibitors, such as those described in Dai W, Zhang B, Jiang X-M, et al. Structure-based design of antiviral drug candidates targeting the SARS-CoV-2 main protease. Science. 2020; 368(6497):1331-1335, including compound designated as DC402234; and/or
antivirals such as remdesivir, galidesivir, favilavir/avifavir, molnupiravir (MK-4482/EIDD 2801), AT-527, AT-301, BLD-2660, favipiravir, camostat, SLV213 emtrictabine/tenofivir, clevudine, dalcetrapib, boceprevir, ABX464, (3S)-3-({N-[(4-methoxy-1H-indol-2-yl)carbonyl]-L-leucyl}amino)-2-oxo-4-[(3S)-2-oxopyrrolidin-3-yl]butyl dihydrogen phosphate; and/or a pharmaceutically acceptable salt, solvate or hydrate thereof (PF-07304814), (1R,2S,5S)—N-{(1S)-1-Cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide or a solvate or hydrate thereof (PF-07321332), and/or S-217622, glucocorticoids such as dexamethasone and hydrocortisone, convalescent plasma, a recombinant human plasma such as gelsolin (Rhu-p65N), monoclonal antibodies such as regdanvimab (Regkirova), ravulizumab (Ultomiris), VIR-7831/VIR-7832, BRII-196/BRII-198, COVI-AMG/COVI DROPS (STI-2020), bamlanivimab (LY-CoV555), mavrilimab, leronlimab (PRO140), AZD7442, lenzilumab, infliximab, adalimumab, JS 016, STI-1499 (COVIGUARD), lanadelumab (Takhzyro), canakinumab (Ilaris), gimsilumab and otilimab, antibody cocktails such as casirivimab/imdevimab (REGN-Cov2), recombinant fusion protein such as MK-7110 (CD24Fc/SACCOVID), anticoagulants such as heparin and apixaban, IL-6 receptor agonists such as tocilizumab (Actemra) and/or sarilumab (Kevzara), PlKfyve inhibitors such as apilimod dimesylate, RIPK1 inhibitors such as DNL758, DC402234, VIP receptor agonists such as PB1046, SGLT2 inhibitors such as dapaglifozin, TYK inhibitors such as abivertinib, kinase inhibitors such as ATR-002, bemcentinib, acalabrutinib, losmapimod, baricitinib and/or tofacitinib, H2 blockers such as famotidine, anthelmintics such as niclosamide, furin inhibitors such as diminazene.

For instance, in one embodiment treatment is selected from a group consisting of (3S)-3-({N-[(— R4-methoxy-1H-indol-2-yl)carbonyl]-L-leucyl}amino)-2-oxo-4-[(3S)-2-oxopyrrolidin-3-yl]butyl dihydrogen phosphate, and a pharmaceutically acceptable salt, solvate or hydrate thereof (PF-07304814). In another embodiment, treatment includes (1R,2S,5S)—N-{(1S)-1-Cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide or a solvate or hydrate thereof (PF-07321332).

In continuation with FIG. 2 and system 200, the presentation component 220 of system 200 may generally be responsible for providing detected respiratory condition information, user instructions and/or feedback for obtaining user voice data and/or self-reported data, and related information. Presentation component 220 may comprise one or more applications or services on a user device, across multiple user devices, or in the cloud environment. For example, in one embodiment, presentation component 220 may manage the provision of information, such as notifications and alerts, to a user across multiple user devices associated with that user. Based on presentation logic, context, and/or other user data, presentation component 220 may determine through which user device(s) content is provided, as well as the context of the provision, such as how (e.g., format and content, which may be dependent on a user device or context) it is provided, when it is provided or other such aspects of the provision of the information.

In some embodiments, presentation component 220 may generate user interface features associated with or used to facilitate presenting aspects of other components of system 200, such as user voice monitor 260, user-interaction manager 280, respiratory-condition tracker 270, and decision support tool(s) 290, to the user (who may be the individual being monitored or a clinician of the monitored individual). Such features may include graphical or audio interface elements (such as icons or indicators, graphics buttons, sliders, menus, sound, audio prompts, alerts, alarms, vibrations, pop-up windows, notification bar or status bar items, in-app notifications, or other similar features for interfacing with a user), queries, and prompts. Some embodiments of presentation component 220 may employ speech synthesis, text-to-speech, or similar functionality for generating and presenting speech to the user, such as embodiments operating on a smart speaker. Examples of graphic user interfaces (GUIs) and representations of example audio user interface elements that may be generated and provided to a user (i.e., a monitored individual or clinician) by presentation component 220 are described in connection with FIGS. 5A-5E. Embodiments utilizing audio user interface functionality are depicted in the examples of FIGS. 4C-4F. Some embodiments of an audio user interface provided by presentation component 220 comprise a voice user interface (VUI), such as the VUI on smart speakers. Examples of graphic user interfaces (GUIs) and representations of example audio user interface elements that may be generated and provided to a user (i.e., a monitored individual or clinician) by presentation component 220 are also shown and described in connection with a wearable device, such as a smartwatch 402a in FIG. 4B.

Storage 250 of example system 200 may generally store information including data, computer instructions (e.g., software program instructions, routines, or services), logic, profiles, and/or models used in embodiments described herein. In an embodiment, storage 250 may comprise a data store (or a computer data memory), such as data store 150 of FIG. 1. Further, although depicted as a single data store component, storage 250 may be embodied as one or more data stores or in the cloud environment.

As shown in the example system 200, storage 250 includes voice-phoneme extraction logic 233, phoneme-features comparison logic 235, and user-condition inference logic 237, all of which are described previously. Further, storage 250 may include one or more individual records (such as individual record 240, as shown in FIG. 2). Individual record 240 may include information associated with a particular monitored individual/user, such as profile/health data (EHR) 241, voice samples 242, phoneme feature vectors 244, results/inferred conditions 246, user account(s)/device(s) 248, and settings 249. The information stored in individual record 240 may be available to data collection component 210, user voice monitor 260, user-interaction manager 280, respiratory-condition tracker 270, decision support tool(s) 290, or other components of the example system 200, as described herein.

Profile/health data (EHR) 241 may provide information relating to a monitored individual's health. Embodiments of profile/health data (EHR) 241 may include a portion or all of the individual's EHR or only some health data that is related to respiratory conditions. For instance, profile/health data (EHR) 241 may indicate past or currently diagnosed conditions, such as influenza, rhinovirus, COVID-19, chronic obstructive pulmonary disease (COPD), asthma or conditions impacting the respiratory system; medications associated with treating the respiratory conditions or with potential symptoms of the respiratory conditions; weight; or age. Profile/health data (EHR) 241 may include the user's self-reported information, such as self-reported symptoms as described in conjunction with self-reporting tools 284.

Voice samples 242 may include raw and/or processed voice-related data, such as data received from sensor(s) 103 (shown in FIG. 1). This sensor data may include data used for respiratory infection tracking, such as the collected voice recordings or samples. In some instances, the voice samples 242 may be stored temporarily until feature vector analysis is performed on the collected samples and/or until a pre-determined period of time has passed.

Further, phoneme feature vectors 244 may include the determined phoneme features and/or phoneme feature vectors for a particular user. Phoneme feature vectors 244 may be correlated to other information in the individual record 240, such as contextual information or self-reported information or composite symptom scores (which may be part of profile/health data (EHR) 241). Additionally, phoneme feature vectors 244 may include information for establishing a phoneme-feature baseline for the particular user as described in conjunction with phoneme-features comparison logic 235.

Results/inferred conditions 246 may comprise user forecasts and inferred respiratory conditions of the user. Results/inferred conditions 246 may be an output by respiratory condition inference engine 278 and, as such, may comprise scores and/or likelihood of the monitored user's respiratory condition presently or in a future time interval. The results/inferred conditions 246 may be utilized by decision support tool(s) 290 as previously described.

User account(s)/device(s) 248 may generally include information about user computing devices accessed, used, or otherwise associated with a user. Examples of such user devices may include user devices 102a-n of FIG. 1 and, as such, may include smart speakers, mobile phones, tablets, smartwatches, or other devices that have integrated voice recording capabilities or that may be communicatively connected to such devices.

In one embodiment, user account(s)/device(s) 248 may include information related to accounts associated with a user, for example, online or cloud-based accounts (e.g., online health record portals, a network/health provider, network websites, decision support applications, social media, email, phone, e-commerce websites, or the like). For example, user account(s)/device(s) 248 may include a monitored individual's account for a decision support application, such as decision support tool(s) 290; an account for a care provider site (which may be utilized to enable electronic scheduling of appointments, for example); and online e-commerce accounts, such as Amazon.com® or a drugstore (which may be utilized to enable online ordering of treatments, for example).

Additionally, user account(s)/device(s) 248 may also include a user's calendar, appointments, application data, other user accounts, or the like. Some embodiments of user account(s)/device(s) 248 may store information across one or more databases, knowledge graphs, or data structures. As described previously, the information stored in the user account(s)/device(s) 248 may be determined from data collection component 210.

Further, settings 249 may generally include user settings or preferences associated with one or more steps for monitoring user voice data, including collecting voice data, collecting self-reported information, or inferring and/or predicting a user's respiratory condition, or one or more decision support applications, such as decision support tool(s) 290. For example, in one embodiment, settings 249 may include configuration settings for collecting voice-related data, such as settings for collecting voice information as the user speaks casually. Settings 249 may include configurations or preferences for contextual information, including settings for obtaining physiological data (e.g., information linking a wearable sensor device). Settings 249 may further include privacy settings, as described herein. Some embodiments of settings 249 may specify specific phonemes or phoneme features to detect or monitor respiratory condition and may further specify detection or inference thresholds (e.g., a condition-change threshold). Settings 249 may also include configurations for users to set a baseline state of their respiratory condition, as described herein. By way of example, and not limitation, other settings may include user notification tolerance thresholds, which may define when and how a user would like to be notified of a user's respiratory condition determination or prediction. In some aspects, settings 249 may include user preferences for applications, such as notifications, preferred caregivers, preferred pharmacy or other stores, and over-the-counter medications. Settings 249 may include an indication of treatment for a user, such as prescribed medication. In one embodiment, calibration, initialization and settings of the sensor(s) (such as sensor 103 described in FIG. 1) may also be stored in settings 249.

Turning now to FIG. 3A, a diagrammatic representation is depicted of an example process 3100 incorporating at least some of the components of system 200. Example process 3100 shows one or more users 3102 providing data via a voice-symptom application 3104, which may operate on a user device, such as a smart mobile device and/or a smart speaker. The data provided via voice-symptom application 3104 may include sound recordings (e.g., voice samples 242 of FIG. 2) from which phonemes may be extracted, as described with respect to user voice monitor 260 in FIG. 2. Additionally, the data received include symptom rating values, which may be manually input by a user, as described in conjunction with user-interaction manager 280.

Based on receiving the recorded voice samples and symptom values, a computer system, which may reside on a server (e.g., server 106 of FIG. 1) and be accessed over a network (e.g., network 110 of FIG. 1), may perform operations 3106 including communicating with the user, performing a symptom algorithm, extracting voice features, and applying a voice algorithm. Communicating with the user may include providing prompts and feedback to collect useable data as described in conjunction with user-interaction manager 280. The symptom algorithm may include generating a composite symptom score (CSS) based on a user's self-reported symptom values, as described in conjunction with self-reporting data evaluator 276. Voice feature extraction may include extracted acoustic feature values for the detected phonemes in the voice samples, as described in conjunction with user voice monitor 260 and, more specifically, acoustic feature extractor 2614. A voice algorithm may be applied to the extracted acoustic features, which may include comparing feature vectors for an individual from different days (i.e., computing a distance metric), as described in conjunction with phoneme features comparer 274.

Based on at least some operations 3106, reminders and notifications may be electronically sent to one or more users 3102 via a user device, such as user device 102a in FIG. 1. Reminders may remind a user to know that a voice sample or additional information, such as self-reported symptom ratings, may be needed. Notifications may provide a user with feedback when providing voice samples, such as indicating whether a longer duration, louder volume, or less background noise is needed or not, as described with respect to user-interaction manager 280. Notifications may also indicate whether and to the extent to which the user has followed the prescribed protocols for providing voice samples and, in some instances, symptom information. For example, a notification may indicate that a user has completed 50% of the voice exercises to provide voice samples.

Additionally, based on at least some of operations 3106, collected information and/or resulting analysis thereof may be sent to one or more user devices associated with a clinician, such as clinician user device 108 in FIG. 1. A clinician dashboard 3108 may be generated by a computer software application, such as decision support app 105a or 105b, operating on or with clinician user device 108 (in FIG. 1). Clinician dashboard 3108 may comprise a graphic user interface (GUI) that enables accessing and receiving information about a specific patient or a set of patients being monitored (i.e., monitored users 3102) and, in some embodiments, communicate directly or indirectly with the patients. Clinician dashboard 3108 may include a view that presents information for multiple users (such as a chart where each row contains information about a different user). Additionally, or alternatively, clinician dashboard 3108 may present information for a single user being monitored.

In one embodiment, clinician dashboard 3108 may be utilized by clinicians to monitor the data collection of users 3102 via voice-symptom application 3104. For example, clinician dashboard 3108 may indicate whether a user has been providing useable voice samples and, in some embodiments, symptom severity ratings or not. Clinician dashboard 3108 may notify a clinician if a user is not adhering to a prescribed protocol for providing voice samples and/or other information. In some embodiments, clinician dashboard 3108 may include functionality to enable a clinician to communicate (e.g., send an electronic message) to a user with a reminder to follow the protocol for collecting data or to follow a revised protocol.

In some embodiments, operations 3106 may include determining a user's respiratory condition (e.g., determining whether the user is sick or not) from the collected voice samples, which may be performed by an embodiment of respiratory-condition tracker 270 generally and, more specifically, respiratory condition inference engine 278, as described in conjunction with FIG. 2. In these embodiments, notifications may be sent to users 3102 indicating a determined respiratory condition. In some embodiments, the notifications to users 3102 may include a recommendation for action, as described in conjunction with decision support tool(s) 290. Further, where the user's voice-related information is utilized to determine the user's respiratory condition, some embodiments of clinician dashboard 3108 may be utilized by a clinician to track user's respiratory condition. Some embodiments of clinician dashboard 3108 may indicate a status of the user's respiratory condition (e.g., a respiratory-condition score, whether or not the user has a respiratory infection), and/or a trend in the user's condition (e.g., whether or not the user's condition is worsening, improving, or staying the same). Alerts or notifications may be provided to a clinician to indicate whether a user's condition is particularly bad (such as when a respiratory-condition score is below a threshold score), whether a new infection is detected for a user, and/or whether a user's condition has changed.

In some embodiments, clinician dashboard 3108 may be utilized to specifically monitor users who have been prescribed a medication for a respiratory infection and/or have been diagnosed by the clinician with a respiratory condition so that the clinician may monitor the condition and the efficacy of prescribed treatment, including side effects of such treatment, as discussed with respect to decision support tool(s) 290 and medication efficacy tracker 296. As such, embodiments of clinician dashboard 3108 may identify a prescribed medication or treatment and whether or not the user is taking the prescribed medication or treatment.

Further, in some embodiments, clinician dashboard 3108 may include functionality to enable a clinician to set a recommended or required voice-sample collection protocol (e.g., how often a user shall provide voice samples), a user's prescribed treatment or medications, and additional recommendations for a user (such as whether or not to drink fluids, get rest, avoid exercise, self-quarantine, for example). Clinician dashboard 3108 may also be used by a clinician to set or adjust monitoring settings (e.g., set thresholds for generating alerts to the clinician and, in some embodiments, to the user). Clinician dashboard 3108 may, in some embodiments, also include functionality to enable a clinician to determine if voice-symptom application 3104 is operating properly and to perform diagnostics on voice-symptom application 3104.

FIG. 3B illustratively depicts a diagrammatic representation of an example process 3500 for collecting data for monitoring respiratory condition. In this example process 3500, monitored individuals may perform several collection checkpoints at which voice samples and symptom ratings are provided. The collection checkpoints may include one in-lab “sick” visit during which time the individual is already experiencing symptoms of a respiratory infection or, in some embodiments, has a respiratory infection diagnosis, and one in-lab “well” visit in which the individual has recovered from the respiratory infection. Additionally, the individual may have a twice-daily (or daily or periodic) collection checkpoints at home between the two in-lab visits. The at-home checkpoints may occur over a period of at least two weeks and may be longer if the individual's recovery time is longer than two weeks. During each collection checkpoint, the individual may provide voice samples and rate symptoms.

The in-lab visits may be a visit with a clinician, such as at a clinician's office or in a lab conducting a study. During the in-lab visits, the monitored individual's voice samples may be recorded simultaneously through a smartphone and a computer coupled to a headset. However, it is contemplated that embodiments of process 3500 may utilize only one of these methods for collecting voice samples during in-lab visits. The individuals may record voice samples and provide symptom ratings, utilizing a smartphone, smartwatch and/or smart speaker for the in-home collections.

For the voice samples in both in-lab visits and in-home visits, individuals may be prompted to record sustained phonations of both nasal consonants and cardinal vowels for 5-10 seconds each. In one embodiment, four vowel sounds, and three nasal constants are recorded. The four vowels using the International Phonetic Alphabet (IPA) may be /a/, /i/, /u/, and /ae/, where individual may be prompted to pronounce sounds using the more vernacular cues “o”, “E”, “OO”, and “a”. The three nasal consonants may be /n/, /m/ and /ng/. In addition, individuals may be asked to record scripted speech and unscripted speech. Voice recording systems may use non-lossy compression and have a bit depth of 16. In some embodiments, voice data may be sampled at 44.1 kilohertz (kHz). In another embodiment, voice data may be sampled at 48 kHz.

During the in-home recovery period, individuals may be asked to provide voice samples and report symptoms every morning and every evening. For the symptom ratings during the at-home period, individuals may be asked to rate their perceived symptom severity (0-5) for 19 symptoms in the morning and 16 symptoms in the evening related to respiratory tract illness. In one embodiment, four sleep questions are included only in the morning list, and an end-of-the-day tiredness question is asked only in the evenings. An example list of symptom questions may be provided in conjunction with self-reporting tools 284. A composite symptom score (CSS) may be determined by summing the scores of at least some of the symptoms. In one embodiment, the CSS is a sum of 7 symptoms (post-nasal discharge, nasal obstruction, runny nose, thick nasal discharge with mucus, cough, sore throat, and need to blow nose).

FIGS. 4A-4F each illustratively depict example scenarios of an individual (i.e., a user 410) utilizing embodiments of the present disclosure. User 410 may interact with one or more user interfaces (e.g., a graphical user interface and/or a voice user interface), as described with respect to presentation component 220 in FIG. 2, of a computer-software application (e.g., decision-support application 105a in FIG. 1) running on a user device (e.g., any of the user computer devices 102a-n). Each scenario is represented by a sequence of scenes (boxes) that are intended to be ordered chronologically (from left to right). Different scenes (boxes) may not necessarily be different discrete interactions but may be portions of one interaction between user 410 and a user interface component.

FIGS. 4A, 4B, and 4C depict data, such as user's voice information being collected from user 410 through interactions with an app or program running on one or more user devices, such as an embodiment of voice-symptom application 3104 in FIG. 3A and/or respiratory-infection monitor app 5101 in FIGS. 5A-5E, as discussed below. Embodiments depicted in FIGS. 4A-4C may be performed by one or more components of system 200, such as user-interaction manager 280, data collection component 210, and presentation component 220.

Turning to FIG. 4A, for example, in a scene 401, user 410 using a smartphone 402c (which may be an embodiment of user device 102c in FIG. 1) is provided instructions 405 for providing a sustained phonation. Instructions 405 state: “Let's begin your voice-condition assessment. Please say and hold the sound ‘mmm’ for 5 seconds, starting now.” These instructions 405 may be provided by an embodiment of user-instruction generator 282 of FIG. 2. The instructions 405 may be displayed as text via a graphical user interface on a display screen of smartphone 402c. Additionally, or alternatively, the instructions 405 may also be provided as audible instructions to utilize a voice user interface on smartphone 402c. In scene 402, user 410 is shown providing voice sample 407 by verbally stating “mmmmmmmm . . . ” on smartphone 402c, such that a microphone (not shown) in the smartphone 402c may pick up and record voice sample 407.

FIG. 4B similarly depicts, in a scene 411, instructions 415 being provided to user 410. Instructions 415 may be generated by an embodiment of user-instruction generator 282 and are provided via a smartwatch 402a, which may be an example embodiment of user device 102a in FIG. 1. As such, instructions 415 may be displayed as text via a graphical user interface on smartwatch 402a. Additionally, or alternatively, the instructions 415 may be provided as audible instructions via a voice user interface. In scene 412, user 410 responds to instructions 415 by speaking to smartwatch 402a that generates voice sample 417 (“aaaaaaaa . . . ”).

FIG. 4C depicts user 410 being guided to provide a voice sample by a series of instructions (which may also be referred to as prompts) from a smart speaker 402b, which may be an embodiment of user device 102b in FIG. 1. The instructions may be output from smart speaker 402b via a voice user interface, and response from user 410 may be audible responses picked up by a microphone (not shown) on smart speaker 402b or another device communicatively coupled to smart speaker 402b.

Additionally, in accordance with some embodiments of this disclosure, FIG. 4C depicts a voice recording session being initiated by an application or program running on or in conjunction with smart speaker 402b. For example, in scene 421, smart speaker 402b states aloud an intention 424 to initiate a voice recording session. Intention 424 states: “Let's begin your voice-condition assessment. Is now a good time?”, to which user 410 provides an audible response 425: “Yes.”.

In scene 422, smart speaker 402b provides audible instructions 426 for user 410 to follow to provide a voice sample, and the user 410 provides audible response 427 that includes a general acknowledgement (“OK”) and the instructed sound (“aaaaa . . . ”). Once it is determined that a user provided a response, it may be determined that the next set of instructions should be given for another voice sample. Determining the response of user 410 and the appropriate feedback to provide user 410 or next steps may be performed by an embodiment of user-input response generator 286. In scene 423, instructions 428 for the next voice sample is emitted from smart speaker 402b, to which user 410 responds with an audible voice sample 429 “mmmmm” This back-and-forth of instructions between smart speaker 402b and user 410 may continue until all of the needed voice samples are collected.

As described herein, a user's respiratory condition may be monitored or tracked utilizing collected voice information from the user. As such, FIGS. 4D, 4E, and 4F depict scenarios in which a user is notified about various aspects of the tracking of the user's respiratory condition. The audio data utilized for the inferences and predictions in FIGS. 4D-4F may be collected over various devices and over different days, such as shown in FIGS. 4A-4C. In some embodiments, the determinations of the inferences and predictions underlying the scenarios in FIGS. 4D-4F may be made by respiratory condition inference engine 278 of FIG. 2, and notifications of such determinations and requests for further information may be provided by embodiments of user-interaction manager 280 and/or decision support tool(s) 290, such as sick monitor 292.

FIG. 4D depicts user 410 being notified of a respiratory condition determination. In scene 431, smart speaker 402b provides an audible message 433 indicating that, based on recent voice data, it is determined that user 410 may be getting sick. This determination that a user may be sick may be made in accordance with embodiments of respiratory-condition tracker 270. Audible message 433 further requests confirmation of symptoms consistent with a respiratory condition (e.g., “Are you feeling congested, tired or . . . ?”), which may be done in accordance with embodiments of self-reporting tools 284 and/or user-input response generator 286. User 410 may provide an audible response 435 “A little.”. In scene 432 in FIG. 4D, a follow-up message 437 is provided by smart speaker 402b in response to user 410's response 435 of feeling congested. The follow-up message 437 requests symptom feedback from the user by asking user 410 to rate the user's congestion. This scenario in FIG. 4D may continue as the user provides a response, rating the user's congestion and/or any other symptoms.

FIG. 4E depicts further interactions between user 410 and smart speaker 402b as the user 410's respiratory condition may be continued to be monitored via user 410's voice data. In an audible message 443 shown in scene 441, smart speaker 402b reminds user 410 that a previously detected respiratory condition (i.e., a cold) is being tracked and notifies user 410 of an updated respiratory condition determination made on more recent data. Specifically, message 443 states: “ . . . Your coughing frequency seems to be decreasing and my analysis of your voice shows improvement. Are you feeling better?”. User 410 then provides audible response 445 indicating that user 410 is feeling better. In scene 442, smart speaker 402b provides an audio message 447 notifying user 410 of a prediction of the user 410's respiratory condition in the future. Specifically, message 447 notifies user 410 that it is predicted that user 410 will be feeling normal with regard to their respiratory condition within three days. Message 447 also provides a recommendation to continue to rest and follow the doctor's orders. The determination that user 410's voice is improving and the determination that a user may be recovered within three days in FIG. 4E may be made by embodiments of respiratory condition inference engine 278, as described in conjunction with FIG. 2.

FIG. 4F depicts a scenario in which the respiratory condition of user 410 is continuing to be monitored (e.g., as indicated by a message 455 in scene 451 stating: “You are still in sickness monitoring mode . . . ”). In scene 451, smart speaker 402b outputs audible message 455 indicating that smart speaker 402b is still in sickness monitoring mode and that user 410 does not appear to be getting better based on analysis of voice samples collected over the last several days. In message 455, smart speaker 402b also asks whether user 410 is taking his antibiotic medication or not. The determination that user 410 is prescribed a medication may be made by an embodiment of prescription monitor 294. User 410 provides response 457 (“Yes.”), indicating that the user 410 is taking the medication. In scene 452, smart speaker 402b communicates over a network to one or more other computing systems or devices, as shown by cloud 458, based on user 410's response 457 confirming that user 410 is taking medication. In one embodiment, smart speaker 402b may be communicating, directly or indirectly, with a care provider of user 410 to refill the user 410's prescription since the user 410 is still sick. Consequently, in scene 453, smart speaker 402b outputs an audible message 459 telling user 410 that the user's care provider has been contacted and a refill of the antibiotic prescription has been ordered.

FIGS. 5A-5E depict various example screenshots from a computing device showing aspects of example graphical user interfaces (GUIs) for a computer software application (or app). In particular, the example embodiments of GUIs depicted in the screenshots of FIGS. 5A-5E (such as a GUI 5100 of FIG. 5A) are for a computer software application 5101, which is referred to as “respiratory-infection monitor app” in these examples. Although the example app depicted in FIGS. 5A-5E is described as monitoring respiratory infections, it is also contemplated that this disclosure similarly applies to an application for monitoring respiratory condition and changes in respiratory condition generally.

Example respiratory-infection monitor app 5101 may include an implementation of user voice monitor 260, user-interaction manager 280, and/or other components or subcomponents, as described in connection with FIG. 2. Additionally, or alternatively, some aspects of respiratory-infection monitor app 5101 may include an implementation of decision support app 105a or 105b and/or may include an implementation of one or more decision support tool(s) 290, as described in connection with FIGS. 1 and 2, respectively. Example respiratory-infection monitor app 5101 may be operating on (and a GUI may be displayed on) a user computing device (or user device) 5102a, which may be embodied as any of user devices 102a-102n, as described in connection with FIG. 1. Some of the GUI elements (such as a hamburger menu icon 5107 of FIG. 5A) of the example GUIs depicted in the screenshots of FIGS. 5A-5E may be selectable by the user, such as by touching or clicking on a GUI element. Some embodiments of user computing device 5102a may comprise a touchscreen or a display operating in conjunction with a stylus or a mouse, for example, to facilitate user interaction with the GUI.

In some aspects, it is contemplated that a prescribed or recommended standard of care for a patient diagnosed with a respiratory condition (e.g., influenza, rhinovirus, COVID-19, asthma or the like) may comprise utilizing an embodiment of the respiratory-infection monitor app 5101, which (as described herein) may operate on the user/patient's own computing device, such as a mobile device, or other user devices 102a-102n, or may be provided to the user/patient via the user/patient's healthcare provider or pharmacy. In particular, conventional solutions to monitor and track respiratory conditions may suffer from being subjective (i.e., from self-tracking symptoms) and either incapable or not practical for early detection, among other deficiencies. But embodiments of the technologies described herein may provide objective, non-invasive, and more accurate means of monitoring, detecting, and tracking respiratory condition data for a user. As a result, these embodiments thereby enable reliable use of technologies for patients who are prescribed certain medicines for respiratory conditions. In this way, a doctor or a healthcare provider may issue an order that may include the user taking medicine and using the computer decision support app (e.g., respiratory-infection monitor app 5101), among other things, track and determine a more precise efficacy of the prescribed treatment. Similarly, doctor or healthcare provider may issue an order that includes (or a standard of care might specify) the patient using the computer decision support app to monitor or track user's respiratory condition prior to taking medication, so that the medicine may be prescribed based on consideration of an analysis, recommendation, or output provided the computer decision support app. For example, the doctor may prescribe a particular antibiotic where the computer decision support app may determine that the user likely has a respiratory condition and does not appear to be recovering. Moreover, the use of the computer decision support app (e.g., respiratory-infection monitor app 5101) as part of the standard of care for a patient who is administered or prescribed a particular medicine supports the effective treatment of the patient by enabling the healthcare provider to better understand the efficacy, including side effects, of the prescribed medicine, modify a dosage or change a particular prescribed medicine, or instruct the user/patient to cease using it since it is no longer needed due to the patient's improving condition.

With reference to FIG. 5A, example GUI 5100 is depicted showing aspects of example respiratory-infection monitor app 5101, which may be used for monitoring a user's respiratory condition and providing decision support. For instance, among other purposes, an embodiment of respiratory-infection monitor app 5101 may be used to facilitate acquiring respiratory-condition data and/or determine, view, track, supplementing, or report information regarding a respiratory condition for a user. The example respiratory-infection monitor app 5101 depicted in GUI 5100 may include a header region 5109, located near the top of GUI 5100, which includes hamburger menu icon 5107, a descriptor 5103, a share icon 5104, a stethoscope icon 5106, and a cycle icon 5108. Selecting hamburger menu icon 5107 may provide the user with access to a menu of other services, features, or functionalities of respiratory-infection monitor app 5101 and may further include access to help, app version information, and secure user-account sign-in/sign-off functionality. Descriptor 5103 may indicate the current date in this example GUI 5100. This date is a date-time that will be associated with any voice-related data acquired by the user if the user is to begin a voice data collection process on this day, as described in connection with a voice analyzer 5120 and FIG. 5B. In some instances, descriptor 5103 may indicate a past date, such as where a user is accessing historical data, a mode or function of respiratory-infection monitor app 5101, a notification for the user, or may be blank.

Share icon 5104 may be selected for sharing, via an electronic communication, various data, analyses or diagnosis, reports, user-provided annotations, or observations (e.g., notes). For example, share icon 5104 may facilitate enabling the user to email, upload, or transmit a report of recent phoneme feature data, respiratory condition changes, inferences or predictions, or other data to a caregiver of the user. In some embodiments, share icon 5104 may facilitate sharing aspects of the various data captured, determined, displayed, or accessed via respiratory-infection monitor app 5101 on social media or with other similar users. In one embodiment, share icon 5104 may facilitate sharing a user's respiratory condition data and, in some instances, related data (e.g., location, historical data, or other information) with a government agency or health department to facilitate monitoring outbreaks of respiratory infection. This shared information may be de-identified to preserve user privacy and encrypted prior to communication.

Selection of stethoscope icon 5106 may provide the user with various communication or connection options to the user's healthcare provider. For example, selecting stethoscope icon 5106 may initiate functionality to facilitate scheduling a tele-appointment (or requesting an in-person appointment), sharing or uploading data to a medical record (e.g., profile/health data (EHR) 241 of FIG. 2) of the user for access by the user's healthcare provider, or accessing a healthcare provider's online portal for additional services. In some embodiments, selecting stethoscope icon 5106 may initiate functionality for the user to communicate specific data, such as the data that the user is currently viewing, to the user's healthcare provider, or may ping the user's healthcare provider to request that the healthcare provider look at the user's data. Finally, selecting cycle icon 5108 may cause a refresh or update to the views and/or data displayed via respiratory-infection monitor app 5101 so that the view is current with regards to the available data. In some embodiments, selecting cycle icon 5108 may refresh data pulled from a sensor (or from a computer application associated with data collection from a sensor, such as sensor(s) 103 in FIG. 1) and/or from a cloud data store (e.g., an online data account) associated with the user.

Example GUI 5100 may also include an icon menu 5110 comprising various user-selectable icons 5111, 5112, 5113, 5114, and 5115, which correspond to various additional functionalities provided by this example embodiment of respiratory-infection monitor app 5101. In particular, selecting these icons may navigate the user to various services or tools provided via the respiratory-infection monitor app 5101. By way of example and without limitation, selecting home icon 5111 may navigate the user to a home screen, which may include a one of the example GUIs described in connection with FIGS. 5A-5E; a welcome screen (such as a GUI 5510 in FIG. 5E), which may include one or more commonly utilized services or tools provided by respiratory-infection monitor app 5101; account information for the user; or any other view (not shown).

In some embodiments, selection of “voice rec” icon 5112, which is shown as being selected in example GUI 5100, may navigate the user to a voice data acquisition mode such as voice analyzer 5120 that comprises application functionality to facilitate acquiring voice samples from the user. Embodiments of voice analyzer 5120 may be performed by one or more components of system 200 including user voice monitor 260 (or one or more of its subcomponents), as described in FIG. 2 and, in some instances, by user-interaction manager 280 (or one or more of its subcomponents), also as described in FIG. 2. For example, functionality of voice analyzer 5120 for acquiring user voice sample data may be carried out as described in connection with voice sample collector 2604.

In some embodiments, voice analyzer 5120 may provide instructions to guide the user through a voice data collection process, such as shown in FIG. 5A on GUI element 5105 and described further in connection with FIG. 5B. In particular, GUI element 5105 depicts aspects of a Repeat Sounds Exercise that prompts a user to repeat a sound for a set duration of time. Here, for example, the user is requested to say the “mmm” sound for 5 seconds. In some embodiments, instructions provided by voice analyzer 5120 may be determined or generated in accordance with user-interaction manager 280 or one or more of the subcomponents, such as user-instruction generator 282.

Descriptor 5103 indicates the current date, which will be associated with the collected voice sample. A timer (a GUI element 5122) may be provided to facilitate instructing the user when to begin or end recording the voice sample. A visual voice sample recording indicator (a GUI element 5123) also may be displayed to provide feedback to user regarding the voice sample recording. In an embodiment, the operations for GUI elements 5122 and 5123 are performed by user-input response generator 286 described in connection with FIG. 2. Other visual indicators (not shown) may include, without limitation, background noise level, mic level, volume, progress indicators, or other indicators described in connection with user-input response generator 286.

In some embodiments (not shown), voice analyzer 5120 may display progress of the user with regards to acquiring voice-related data within a time interval (e.g., for the day or half-day). For example, where voice-related data is acquired through casual interaction or by reading a passage, voice analyzer 5120 may depict an indication of the user's progress such as a percentage towards completion, a dial or a sliding progress bar, or an indication of phonemes that have successfully been obtained or not yet obtained from the user's speech. Additional GUIs and details for an example voice data collection process performed by voice analyzer 5120 are described in connection with FIG. 5B.

Referring again to FIG. 5A in continuation with GUI 5100 and icon menu 5110, selecting outlook icon 5113 may navigate the user to a GUI and functionality for providing the user with tools and information about the user's respiratory condition. This may include, for example, information about the user's current respiratory condition(s), trend(s), forecast(s), or recommendation(s). Additional details of the functionality associated with outlook icon 5113 are described in connection with FIG. 5C. Selecting log icon 5114 (FIG. 5A) may navigate the user to a log tool that comprises functionality to facilitate respiratory condition tracking or monitoring, such as described in connection with FIGS. 5D and 5E. In an embodiment, functionality associated with log tool or log icon 5114 may include a GUI and tools or services for receiving and viewing physiological data for the user, symptoms data, or other contextual information. For example, one embodiment of a log tool comprises a self-reporting tool for logging user symptoms, such as described in connection with FIGS. 5D and 5E.

In some embodiments, selecting settings icon 5115 may navigate the user to a user-setting configuration mode that may enable specifying various user preferences, settings, or configurations of respiratory-infection monitor app 5101, aspects of voice-related data (e.g., sensitivity thresholds, phoneme-feature comparison settings, configurations regarding phoneme features, or other settings regarding the acquisition or analysis of voice-related data), user account(s), information about the user's care provider(s), caregiver(s), insurance, diagnosis or conditions, user care/treatment, or other settings. In some embodiments, at least a portion of settings may be configured by the user's healthcare provider or a clinician. Some settings accessible via settings icon 5115 may include settings discussed in connection with settings 249 of FIG. 2.

Turning now to FIG. 5B, a sequence 5200 is provided of example GUIs 5210, 5220, 5230, and 5240, showing aspects of an example process for acquiring voice-related data in which a user is guided to provide voice samples of various vocalizations. The process depicted in the GUIs of sequence 5200 may be provided by respiratory-infection monitor app 5101 operating on user computing device 5102a, which may display GUIs 5210, 5220, 5230, and 5240. In an embodiment, the functionality depicted in GUIs 5210, 5220, 5230, and 5240 is provided by a voice data acquisition mode of respiratory-infection monitor app 5101, such as voice analyzer 5120 described in FIG. 5A, and may be accessed or initiated by selecting voice rec icon 5112 of GUI 5100 (FIG. 5A). The instructions depicted in GUIs 5210, 5220, 5230, and 5240 for guiding the user (e.g., instructions 5213) may be determined or generated in accordance with user-interaction manager 280 or one or more of the subcomponents, such as user-instruction generator 282.

As shown in GUI 5210, instructions 5213 are shown guiding the user to vocalize a succession of sounds as part of a repeat sounds exercise. The repeat sounds exercise may comprise one or more vocalization tasks to be performed by the user. In this example, the user may begin the exercise (or a task within the exercise) by selecting a start button 5215. GUI 5210 also depicts a progress indicator 5214, which is a sliding bar indicating the user's progress (e.g., 60% complete) towards providing voice sample data for this session or time interval.

GUIs 5220, 5230, and 5240 continue to depict aspects of guiding a user to vocalize a succession of sounds as part of the repeat sounds exercise. As shown in sequence 5200, example GUIs 5220, 5230, and 5240 include various visual indicators to facilitate guiding the user or providing feedback to the user. For instance, GUI 5220 includes GUI element 5222, which shows a countdown timer and indicator of background noise checking. The countdown timer of GUI element 5222 indicates the time until a user should begin the vocalization. GUI 5230 includes GUI element 5232, which shows another example of a timer, which, in this instance, indicates a duration of time that the user has sustained vocalizing the “ahhh” sound. Similarly, GUI 5240 includes GUI element 5242 that shows an example of a timer, which, in this instance, indicates that the user has vocalized the “mmm” sound for 5 seconds. GUI 5240 also includes a GUI element 5243 providing feedback to the user regarding the voice sample recording for the “mmm” sound. As described previously, functionality associated with visual indicators such as progress indicator 5214, the countdown timer and background noise indicator of GUI element 5222, the timers of GUI elements 5232 and 5242, or voice sample recording indicator of GUI element 5243 may be provided by user-input response generator 286. Additional examples of visual indicators and user feedback operations that may be provided are described in connection with user-input response generator 286.

In continuation with sequence 5200, GUI 5240 may represent a final stage of the repeat sounds exercise for acquiring voice sample data or may represent the end of one stage among multiple stages of a process for acquiring voice sample data. For instance, there may be additional vocalization tasks or exercises to be performed subsequently. Upon providing a voice sample, the user may end the exercise (or a task within the exercise) by selecting a complete button 5245. Alternatively, if the user desires to redo the task and provide another voice sample, the user may select a GUI element 5244 to start the task over again. In some embodiments, a user may be provided an indication or instruction to redo the task, such as where the voice sample is determined to be deficient, as described in connection with sample recording auditor 2608 and user-input response generator 286.

The example process shown in sequence 5200 for collecting voice-related data involves prompting a user with instructions as part of a repeat sounds exercise. However, other embodiments of respiratory-infection monitor app 5101 may acquire voice-related data from casual interaction, as described herein. Further, in some embodiments voice-related data may be collected from a combination of casual interactions and from a repeating sounds exercise, such as the example in FIG. 5B. For instance, where casual interaction has not yielded enough or the specific type of usable voice-related data for a given time interval (e.g., for that day or half-day), then a user may be notified (e.g., via respiratory-infection monitor app 5101) to provide the additional voice-related data via a repeat sounds exercise or similar interaction. In some embodiments, the user may configure options for how their voice-related data may be acquired, such as via settings icon 5115 or as described in connection with settings 249 of FIG. 2.

Turning now to FIG. 5C, another aspect of respiratory-infection monitor app 5101 is depicted including a GUI 5300. GUI 5300 includes various user-interface (UI) elements for displaying a user's respiratory condition outlook (e.g., outlook 5301), and the functionality depicted in GUI 5300 may be accessed or initiated by selecting outlook icon 5113 of GUI 5100 (FIG. 5A). Example GUI 5300 further includes a descriptor 5303 indicating a current date that the user is accessing the outlook functionality of respiratory-infection monitor app 5101 (e.g., Today, May the 4th) and user's outlook 5301, indicating that the user is in the outlook mode of operation (or is accessing the outlook functionality) of respiratory-infection monitor app 5101. As shown in FIG. 5C, icon menu 5110 indicates that the outlook icon 5113 is selected, which may present the user with GUI 5300, depicting the user's outlook 5301. Outlook 5301 may include respiratory condition determinations and/or forecasts and related information for the user. For example, outlook 5301 may include a respiratory-condition score 5312, a transmission risk 5314 which may include related recommendations 5315, and a trend information, such as trend descriptor 5316 and a GUI element 5318.

As described herein, respiratory-condition score 5312 may quantify or characterize a user's respiratory condition, which may represent the user's current respiratory condition, a change in the user's respiratory condition, or the user's likely future respiratory condition. As further described herein, the respiratory-condition score 5312 may be based on the user's voice-related data, such as voice-related data acquired through the example process shown in FIG. 5B or described in connection with user voice monitor 260 in FIG. 2. In some instances, the respiratory-condition score 5312 further may be based on contextual information such as user observations (e.g., self-reported symptom scores), health or physiological data (e.g., data provided by a wearable sensor or the user's health record), weather, location, community infection information (e.g., current infection rate in the user's geographic location), or other contexts. Additional details of determining respiratory-condition score 5312 are provided in connection with respiratory condition inference engine 278 of FIG. 2 and method 6200 of FIG. 6B.

Transmission risk 5314 in GUI 5300 may indicate a risk of the user transmitting a detected respiratory-related infectious agent. Transmission risk 5314 may be determined as described in connection with respiratory condition inference engine 278 and user-condition inference logic 237 of FIG. 2. The transmission risk may be a quantitative or categorical indicator, such as “med-high” indicating a medium-to-high risk in the example GUI 5300. Along with transmission risk 5314, outlook 5301 may provide recommendations 5315, which may include recommended practices to reduce the risk of transmission, such as wearing a face mask, social distancing, self-quarantining (staying home), or consulting a healthcare provider.

These recommendations 5315 may comprise pre-determined recommendations and, in some embodiments, may be determined based on the particular detected respiratory condition and/or the transmission risk 5314 according to a set of rules. In some embodiments, recommendations 5315 may be tailored for the user based on the user's historical information, such as historical voice-related information, and/or contextual information, such as geographical location. Additional details for determining recommendations 5315 are described in connection with respiratory condition inference engine 278 and user-condition inference logic 237 of FIG. 2.

Outlook 5301 may provide trend information, such as trend descriptor 5316 and, in some embodiments, GUI element 5318 that provides a visualization of the trend or change in the user's respiratory condition over time. Trend descriptor 5316 may indicate previously or currently detected changes to a user's respiratory condition. Here, the trend descriptor 5316 states that a user's respiratory condition is getting worse. Further, GUI element 5318 may include a graph or chart of the user's data, or other visual indication showing changes to user respiratory condition, such as changes to phoneme features detected from voice samples over the past 14 days. In other embodiments, outlook 5301 additionally or alternatively provides a forecast of a likely trend in the user's respiratory condition in the future. For example, GUI element 5318 may, in some embodiments, indicate future dates and predict future changes in the user's respiratory condition as described with respect to respiratory condition inference engine 278. In one embodiment, outlook 5301 provides a forecast indicating when the user is likely to be recovered from a respiratory infection (e.g., “You should feel normal within 3 days.”). Another example forecast that may be provided by outlook 5301 comprises an early-warning forecast, such as upon the first detection of a likely respiratory infection, a forecast indicating that the user might expect to be sick at a future time interval (e.g., “You appear to be developing a respiratory infection and may feel sick by the end of the week.).

In some instances, respiratory-infection monitor app 5101 may generate or provide an electronic notification to the user (or caregiver or clinician) regarding the forecast or regarding other information provided by outlook 5301. Information provided by outlook 5301, which may include trend or forecast information utilized for generating trend descriptor 5316 and/or GUI element 5318, may be determined by an example embodiment of respiratory-condition tracker 270 or one or more of its subcomponents, such as respiratory condition inference engine 278 in FIG. 2. Additional details of determining respiratory condition information, transmission risk 5314, recommendations 5315, forecasts, or trend information 5316 are described in connection with respiratory-condition tracker 270 in FIG. 2.

Turning now to FIG. 5D, another aspect of respiratory-infection monitor app 5101 is depicted including a GUI 5400. GUI 5400 includes UI elements for displaying or receiving respiratory-condition related information (such as respiratory symptoms) and corresponds to the log functionality indicated by log icon 5114. In particular, GUI 5400 depicts an example of a log tool 5401 for logging, viewing, and, in some aspects, annotating current or historical user data. Log tool 5401 may be accessed by selecting the log icon 5114 from icon menu 5110. In some embodiments, log tool 5401 (or a self-reporting tool 5415, described below) may be presented to the user (or the user may receive a notification to access log tool 5401) upon a determination that the user is or may have a respiratory infection. Example GUI 5400 further includes a descriptor 5403 indicating that the information displayed by log tool 5401 is for the date Monday, May 4. In some embodiments of log tool 5401, a user may navigate to a previous date to access historical data, for example by selecting a date arrow 5403a or by selecting history tab 5440 and then selecting a particular calendar date from a calendar view (not shown).

As shown in this example GUI 5400 of respiratory-infection monitor app 5101, log tool 5401 includes five selectable tabs: add symptoms 5410, notes 5420, reports 5430, history 5440, and treatment 5450. These tabs may correspond to additional functionality provided by log tool 5401. For example, as shown in GUI 5400, the tab for add symptoms 5410 is selected, and thus, various UI components are presented for a user to self-report symptoms that may be related to their respiratory condition. In particular, the functionality corresponding to add symptoms 5410 comprises a self-reporting tool 5415 that includes a list of symptoms and user-selectable sliders for receiving user input regarding the severity that the user is experiencing each symptom. For example, the self-reporting tool 5415 shown in GUI 5400 depicts that a user is experiencing moderate levels of shortness of breath and congestion and a severe cough. In some embodiments, a user may input this symptom data each day or multiple times a day (e.g., such as every morning and every evening) utilizing self-reporting tool 5415. In some instances, the symptom data may be entered at or near a time interval for collecting voice-related data from the user.

In some embodiments, add symptoms 5410 (or log tool 5401) also may include a selectable option 5412 for the user to input data from another computing device, such as a wearable smart device or similar sensor. For example, a user may select to input data from a fitness tracker so that it may be received by log tool 5401. In some embodiments, the data may be received directly and/or automatically from the smart device or from a database (e.g., an online account) associated with the device. In some instances, a user may need to link or associate the device with their respiratory-infection monitor app 5101 (or with a user account associated with the respiratory-infection monitor app 5101) in order to input the data. In some embodiments, a user may configure various parameters for inputting data from another device in application settings (e.g., by selecting setting icon 5115, as described in FIG. 5A). For example, a user may specify which data is to be inputted (e.g., a user's sleep data acquired by a smartwatch), when the data is to be inputted, or may configure permission settings, account linking, or other settings.

By way of example and without limitation, inputting such data to utilize selectable option 5412 may be utilized in conjunction with or without self-reporting tool 5415. For example, data imported from a linked smart device may provide initial severity ratings for symptoms based on information a user input into the linked smart device, but a user may utilize self-reporting tool 5415 to adjust those initial ratings. Additionally, add symptoms 5410 may include another selectable option 5418 to indicate that symptoms have not changed since the last time the user logged symptoms, such as the previous day. Functionality and UI elements associated with add symptoms 5410 in GUI 5400 may be generated by utilizing an embodiment of user-interaction manager 280 or one or more subcomponents, such as self-reporting tools 284 described in conjunction with FIG. 2.

In continuation with GUI 5400 shown in FIG. 5D, the tab for notes 5420 may navigate the user to functionality for respiratory-infection monitor app 5101 (or, more specifically, log functionality associated with log tool 5401) for receiving or displaying observational data from a user or a caregiver for that particular date (here, May 4). Examples of observational data may include notes 5420 documenting or relating to the user's respiratory condition, such as symptoms. In some embodiments, notes 5420 include a UI for receiving text (or audio or video recordings) from the user. In some aspects, UI functionality for notes 5420 may comprise a GUI element showing a human body configured to receive input from the user indicating areas of the user's body affected by a potential or known respiratory condition, symptoms or side effects. In some embodiments, a user may enter contextual information, such as the user's geographical location, weather, and any physical activity that the user engaged in during the day, for example.

The tab for reports 5430 may navigate the user to a GUI for viewing and generating various reports of the respiratory-condition related data detected by the embodiments described herein. For example, reports 5430 may include a historical or trend information regarding a user's respiratory condition or a prediction of the user's respiratory condition. In another example, reports 5430 may include a report of respiratory-condition information for a larger population. For instance, reports 5430 may show a number of other users of respiratory-infection monitor app 5101 for whom the same or a similar respiratory condition was detected. In some embodiments, functionality provided by reports 5430 may comprise operations for formatting or preparing the respiratory-condition related data to be communicated to or shared with (e.g., via share icon 5104 or stethoscope icon 5106, of FIG. 5A) a caregiver or clinician.

The tab for history 5440 may navigate the user to a GUI for viewing the user's historical data relating to respiratory condition monitoring. For example, selecting history 5440 may display a GUI with a calendar view. The calendar view may facilitate accessing or displaying the detected and interpreted respiratory-condition related data for the user at different dates. For example, by selecting a particular previous date of within a displayed calendar, the user may be presented with a summary of the data for that date. In some embodiments of a calendar view GUI displayed upon selecting the tab for history 5440, indicators or information may be displayed on dates of the calendar, indicating detected or forecasted respiratory-condition information associated with that date.

Selection of the tab indicating a treatment 5450 on GUI 5400 may navigate the user to a GUI within respiratory-infection monitor app 5101 with functionality for the user to specify details such as whether the user took any treatment and/or had any side effects on that date. For example, the user may specify that the user took a prescribed antibiotic or breathing treatment on a particular date. It is also contemplated that, in some embodiments, smart pillboxes or smart containers, which may include so-called internet-of-things (IoT) functionality, may automatically detect that a user has accessed medicine stored within a container and may communicate an indication to respiratory-infection monitor app 5101 indicating that the user took treatment on that date In some embodiments, the tab for treatment 5450 may comprise a UI, enabling the user (or a caregiver or clinician for the user) to specify their treatment, for instance, by selecting check-boxes indicating the kind of treatment the user followed on that date (e.g., took prescription medicine, took over-the-counter medicine, drank plenty of clear fluids, rested, and so on).

Turning to FIG. 5E, a sequence 5500 is provided of example GUIs 5510, 5520, and 5530 showing aspects of an example process for a user-initiated symptom report. GUIs 5510, 5520, and 5530 may be generated in accordance with an embodiment of self-reporting tools 284 described in conjunction with FIG. 2. In some instances, when a user launches respiratory-infection monitor app 5101 on user computing device 5102a, GUI 5510 may be provided as a welcome/login screen. As described herein, respiratory-infection monitor app 5101 may be associated with a particular user, which may be indicated by a user account. As depicted, GUI 5510 includes UI elements for a user to input user credentials (i.e., a user identifier, such as an email address, and a password) to identify the user so that user-specific information may be accessed, and user input may be properly stored in association with the user. Following the user logging in via GUI 5510 and a GUI 5520 may be provided with an initial instruction prompting the user to report symptoms. GUI 5520 may include a selectable “symptom report” button that may cause presentation of a GUI 5530 with UI elements for facilitating input of user symptom information. In the example embodiment of GUI 5530, a user may rate the severity of symptoms by moving a slider to the appropriate severity level for each symptom displayed within GUI 5530. Further details of user-input of symptom information are described with respect to GUI 5400 of FIG. 5D.

FIGS. 6A and 6B depict flow diagrams of example methods utilized in monitoring a user's respiratory condition. FIG. 6A, for example, depicts a flow diagram illustrating an example method 6100 for obtaining phoneme features, in accordance with an embodiment of the disclosure. FIG. 6B depicts a flow diagram illustrating an example method 6200 for monitoring the respiratory condition of a user based on phoneme features, in accordance with an embodiment of the disclosure. Each block or step of methods 6100 and 6200 comprises a computing process that may be performed using any combination of a hardware, a firmware, and/or a software. For instance, various functions may be carried out by a processor executing instructions stored in a memory. The methods may also be embodied as computer-usable instructions stored on computer storage media. The methods may be provided by a stand-alone application, a service or a hosted service (stand-alone or in combination with another hosted service), or a plug-in to another product, to name a few. Accordingly, methods 6100 and 6200 may be performed by one or more computing devices, such as a smartphone or other user device, a server, or a distributed computing platform, such as in the cloud environment. Example aspects of computer program routines covering implementations of phoneme feature extraction are illustratively depicted in FIGS. 15A-M.

Turning to method 6100 of FIG. 6A, method 6100 includes steps for detecting phoneme features, in accordance with an embodiment of the disclosure, and embodiments of method 6100 may be performed by embodiments of one or more components of system 200, such as user voice monitor 260 described in connection with FIG. 2. At step 6110, audio data is received. In some embodiments, step 6110 is carried out by an embodiment of voice sample collector 2604 described in connection with FIG. 2. Additional embodiments of step 6110 are described in connection with voice sample collector 2604 and user voice monitor 260.

The audio data received in step 6110 may include recordings (e.g., audio samples, voice samples) of a user vocalizing individual phoneme sounds or combinations of phonemes, such as scripted or unscripted speech. In this way, the audio data comprises voice information about a user. The audio data may be collected during a user's casual or everyday interaction with a user device, such as user devices 102a-n of FIG. 1, having a sensor (such as an embodiment of sensor(s) 103 of FIG. 1), such as a microphone.

Some embodiments of method 6100 includes operations performed before audio data is received in step 6110. For example, operations for determining a proper or optimized configuration for obtaining usable audio data may be performed, such as determining acoustic parameters for sensors (e.g., microphone) and/or modifying acoustic parameters, such as signal strength, directivity, sensitivity, frequency, and signal to noise ratio (SNR). These operations may be in connection with sound recording optimizer 2602 of FIG. 2. Similarly, these operations may include identifying and, in some aspects, removing or reducing background noise as described in connection with background noise analyzer 2603 of FIG. 2. These steps may include comparing noise intensity levels to a maximum threshold, checking for speech within pre-determined frequencies, and checking for intermitted spikes or similar acoustic artifacts.

In some embodiments, user instructions may be provided to facilitate receiving audio data. For example, a user may be guided through providing audio date by following speech-related tasks. The user instructions may also include feedback based on recently provided samples, such as instructing the user to speak louder or hold a vocalized phoneme for a longer duration. Interactions with the user to facilitate receiving audio data may be carried out by embodiments of user interaction manager 280 generally or its subcomponent user-instruction generator 282 described in connection with FIG. 2.

At step 6120, a date-time value corresponding to the time interval is determined. The date-time value may be the time in which the audio data is received or recorded from the user's vocalization(s). In some embodiments, step 6120 is performed by an embodiment of voice sample collector 2604 described in connection with FIG. 2.

At step 6130, at least a portion of the audio data is processed to determine a phoneme. Some embodiments of step 6130 may be carried out by an embodiment of phoneme segmenter 2610 described in connection with FIG. 2. Determining a phoneme from a portion of the audio data may include performing automatic speech recognition (ASR) on the portion of the audio data to detect a phoneme and associating the detected phoneme with the portion of the audio data. ASR may determine a text (e.g., a word) from a portion of the audio data and the phoneme may be determined based on the recognized text. Alternatively, determining a phoneme may include receiving an indication of a phoneme corresponding to a portion of the audio data and associating the phoneme with the portion of the audio data. This process may be particularly useful where the audio data is of sustained phoneme vocalizations based on speech-related tasks given to the user. For example, a user may be instructed to say “aaa” for 5 seconds, then “eee” for 5 seconds, then “nnnn” for 5 seconds, then “mmm” for 5 seconds”, and those instructions may indicate the order of phonemes (i.e., /a/, /e/, /n/, and /m/) expected for the audio data.

Processing the audio data to determine phonemes may include detecting and isolating the particular phonemes. In one embodiment, phonemes corresponding to /a/, /e/, /u/, /ae/, /n/, /m/, and /ng/ are detected. In another embodiment, only /a/, /e/, /m/, and /n/ are detected. Alternatively, processing the audio data may include detecting what phonemes are present and isolating all detected phonemes. Phonemes may be detected by applying intensity thresholds to separate background noise from the user's voice as described further in conjunction with phoneme segmenter 2610 of FIG. 2.

Some aspects of processing audio data in step 6130 may include additional processing steps, which may be performed by an embodiment of signal preparation processor 2606 of FIG. 2. For example, frequency filtering, such as high-pass or band-pass filtering, may be applied to remove or attenuate frequencies of the audio data that represent background noise. In one embodiment, a band-pass filter of 1.5 to 6.4 kilohertz (kHz) is applied for example. Step 6130 may also include performing audio normalization to achieve a target signal amplitude level(s), SNR improvement through application of band filters and/or amplifiers, or other signal conditioning or pre-processing.

At step 6140, based on the determined phoneme, a phoneme feature set is determined. Some embodiments of step 6140 are carried out by embodiments of acoustic feature extractor 2614 described in conjunction with FIG. 2. The phoneme feature set comprises at least one acoustic feature characterizing the processed portion of the audio data. The feature set may include measures of a power and a power variability, a pitch and a pitch variability, a spectral structure, and/or formants, which are further described in connection with acoustic feature extractor 2614. In some embodiments, different feature sets (i.e., different combinations of acoustic features) are determined for different phonemes detected in the audio data. For example, in an exemplary embodiment, 12 features are determined for the /n/ phoneme, 12 features are determined for the /m/ phoneme, and 8 features are determined for the /a/ phoneme. The feature set for a detected /a/ phoneme may include: standard deviation of formant 1 (F1) bandwidth; pitch interquartile range; spectral entropy determined for 1.6 to 3.2 kilohertz (kHz) frequencies; jitter; standard deviation of mel-frequency cepstral coefficients MFCC9 and MFCC12; mean of mel-frequency cepstral coefficient MFCC6; and spectral contrast determined for 3.2 to 6.4 kHz frequencies. The feature set for a detected /n/ phoneme may include: harmonicity; standard deviation of F1 bandwidth; pitch interquartile range; spectral entropy determined for 1.5 to 2.5 kHz and 1.6 to 3.2 kHz frequencies; spectral flatness determined for 1.5 to 2.5 kHz frequencies; standard deviation of mel-frequency cepstral coefficients MFCC1, MFCC2, MFCC3, and MFCC11; mean of mel-frequency cepstral coefficient MFCC8; and spectral contrast determined for 1.6 to 3.2 kHz frequencies. The feature set for a detected /m/ phoneme may include: harmonicity; standard deviation of F1 bandwidth; pitch interquartile range; spectral entropy determined for 1.5 to 2.5 kHz and 1.6 to 3.2 kHz frequencies; spectral flatness determined for 1.5 to 2.5 kHz frequencies; standard deviation of mel-frequency cepstral coefficients MFCC2 and MFCC10; mean of mel-frequency cepstral coefficients MFCC8; shimmer; spectral contrast determined for 3.2 to 6.4 kHz frequencies; and standard deviation of 200 hertz (Hz) third-octave band. Additionally, in some embodiments, values of one or more features in the feature set may be transformed. In an example embodiment, a log transformation is applied to pitch interquartile range, standard deviation of MFCC, spectral contrast, jitter and standard deviation within the 200 Hz third-octave band.

At step 6155, it is determined whether there is additional audio data to process or not. In some embodiments, step 6155 is carried out by an embodiment of user voice monitor 260. As described, the received audio data may be a recording of multiple sustained phonemes or speech (scripted or unscripted) and, as such, may have multiple phonemes. In this way, different portions of the audio data may be processed to detect different phonemes. For example, a first portion may be processed to determine a first phoneme, a second portion may be processed to determine a second phoneme, and a third portion may be processed to detect a third phoneme, where the first, second, and third phonemes may correspond to /a/, /n/, and /m/, respectively. In some aspects, a fourth portion is processed to detect a fourth phoneme, where the fourth phoneme may be /e/. These phonemes may be recorded by a user vocalizing these three phonemes in one recording. As such, additional audio data in step 6155 may include additional portions of the same voice sample that is already partially processed. In addition, or alternatively, step 6155 may include determining whether there is additional audio data to process or not from additional voice samples recorded in the same session (i.e., acquired in the same time frame). For example, the three phonemes may be recorded in separate recordings from the same session.

If there is additional audio data left to process at step 6155, steps 6130 and 6140 may be performed on the additional audio data portions. FIG. 6A depicts step 6155 occurring after an initial portion of the audio data is processed and a feature set is determined for a detected phoneme; however, it is contemplated that embodiments of method 6100 may include determining whether there is additional audio data to process or not for detection of additional phonemes in step 6155 before any feature sets are extracted.

When there is no additional audio data left to process and feature sets left to determine, method 6100 proceeds to step 6160 where the phoneme feature set extracted from the audio data is stored in a record associated with the user. The stored phoneme feature set includes an indication of the date-time value. In some embodiments, step 6160 is carried out by an embodiment of user voice monitor 260 or, more particularly, acoustic feature extractor 2614. The phoneme feature set may be stored in a user's individual record, such as individual record 240. More particularly, the phoneme feature set may be stored as a vector and stored as phoneme feature vectors 244 in FIG. 2.

Some embodiments of method 6100 include additional operations to monitor a user's respiratory condition over time and, in some aspects, detect a change in a user's respiratory condition. For example, steps 6110 through 6160 may be performed for a first audio data sample recorded for a first time interval, and steps 6110 through 6160 may be repeated for a second audio data sample recorded for a second, subsequent time interval. As such, a first phoneme feature set may be determined and stored for a first time interval and a second phoneme feature set may be determined and stored for a second time interval. Method 6100 may then include operations to utilize the first and second phoneme feature sets to monitor the user's respiratory condition over time. For example, the first and second phoneme feature sets may be compared to detect a change. This comparing operation may be performed by an embodiment of phoneme features comparer 274 and may include determining a feature distance measurement (e.g., Euclidean distance) between feature set vectors for the first and second time intervals. Based on the feature distance measurement (e.g., the magnitude of the measurement and/or whether it is positive or negative), it may be determined whether the user's respiratory condition has changed between the second and first time intervals or not.

In some embodiments, method 6100 further includes receiving contextual information associated with the time interval (e.g., first time interval and/or second time interval) and storing the contextual information in the record in association with the feature set determined for the relevant time interval. These operations may be performed by an embodiment of contextual information determiner 2616 of FIG. 2. The contextual information may include physiological data for the user, which may be self-reported, received from one or more physiological sensors, and/or determined from the user's electronic health record (e.g., profile/health data (EHR) 241 in FIG. 2). Additionally, or alternatively, contextual information may include location information of the user during the relevant time interval or other contextual information associated with the first time interval. Embodiments of step 6140 may include determining the phoneme feature set further determined based on the contextual data for the relevant time interval.

Turning to FIG. 6B, method 6200 includes steps for monitoring the respiratory condition of a user based on phoneme features, in accordance with an embodiment of the disclosure. Method 6200 may be performed by embodiments of one or more components of system 200, such as respiratory-condition tracker 270 described in connection with FIG. 2. Step 6210 includes receiving phoneme feature vectors (which may also be referred to as phoneme feature sets) representing voice information of a user at different times. As such, a first phoneme feature vector (i.e., first phoneme feature set) is associated with a first date-time value, and a second phoneme feature vector (i.e., second phoneme feature set) is associated with a second date-time value that occurs after the first date-time value. For example, the first phoneme feature vector may be based on audio data captured during a first interval (corresponding to the first time-date value) that is within approximately 24 hours (e.g., between 18 to 36 hours) of capturing audio data utilizing to determine the second phoneme feature vector during a second interval (corresponding to the second time-date value). It is contemplated that the time between the first and second time-date values may be less (e.g., 8 to 12 hours) or greater (e.g., three days, five days, one week, two weeks). Step 6210 may be carried out by respiratory-condition tracker 270 generally or, more specifically, feature vector time series assembler 272 or phoneme features comparer 274.

Determination of the first and second phoneme feature vectors may be performed in accordance with an embodiment of method 6100 of FIG. 6A. In some embodiments, determining the first and/or second phoneme feature sets may be done by processing audio information comprising voice information to determine first and/or second set of phonemes and, for each phoneme within the set(s), extracting a set of features that characterize the phoneme. In some embodiments, the first and second feature vectors comprise acoustic feature values characterizing the phonemes /a/, /m/, and /n/. In an exemplary embodiment, the first and second feature vectors each include 8 features for phoneme /a/, 12 features for phoneme /n/, and 12 features for phoneme /m/. The features for phoneme /a/ may include: standard deviation of formant 1 (F1) bandwidth; pitch interquartile range; spectral entropy determined for 1.6 to 3.2 kilohertz (kHz) frequencies; jitter; standard deviation of mel-frequency cepstral coefficients MFCC9 and MFCC12; mean of mel-frequency cepstral coefficient MFCC6; and spectral contrast determined for 3.2 to 6.4 kHz frequencies. The features for phoneme /n/ may include: harmonicity; standard deviation of F1 bandwidth; pitch interquartile range; spectral entropy determined for 1.5 to 2.5 kHz and 1.6 to 3.2 kHz frequencies; spectral flatness determined for 1.5 to 2.5 kHz frequencies; standard deviation of mel-frequency cepstral coefficients MFCC1, MFCC2, MFCC3, and MFCC11; mean of mel-frequency cepstral coefficient MFCC8; and spectral contrast determined for 1.6 to 3.2 kHz frequencies. The features for phoneme /m/ may include: harmonicity; standard deviation of F1 bandwidth; pitch interquartile range; spectral entropy determined for 1.5 to 2.5 kHz and 1.6 to 3.2 kHz frequencies; spectral flatness determined for 1.5 to 2.5 kHz frequencies; standard deviation of mel-frequency cepstral coefficients MFCC2 and MFCC10; mean of mel-frequency cepstral coefficients MFCC8; shimmer; spectral contrast determined for 3.2 to 6.4 kHz frequencies; and standard deviation of 200 hertz (Hz) third-octave band. In some embodiments, one or more of these features are extracted to characterized a /e/ phoneme.

In some embodiments, the first phoneme feature vector determined for a first time interval is based on multiple phoneme feature sets from multiple audio samples captured prior to the second date-time value. The first feature vector may represent a combination, such as an average, of the multiple phoneme feature vectors. These multiple audio samples may be taken from times when an individual is known or presumed to be healthy (i.e., has no respiratory infection) such that the first feature vector may represent a healthy baseline. Alternatively, the audio samples utilized for determining the first phoneme feature vector may be taken from times when the individual is known or presumed to be sick (i.e., has a respiratory infection), and the first phoneme feature vector may represent a sick baseline.

Step 6220 includes performing a comparison of the first and second phoneme feature vectors to determine a phoneme feature-set distance. In some embodiments, step 6220 may be carried out by an embodiment of phoneme features comparer 274 of FIG. 2. In some embodiments, this comparison includes determining a Euclidean distance between the first and second phoneme feature sets. Each feature representing by a feature vector may be compared to a corresponding feature within the other feature vector. For example, a first feature (e.g., jitter for phoneme /a/) in the first phoneme feature vector may be compared to the corresponding feature (e.g., jitter for phoneme /a/) in the second phoneme feature vector.

At step 6230, it is determined that the user's respiratory condition has changed based on the phoneme feature-set distance between the first and second phoneme feature vectors. In some embodiments, step 6230 is performed by an embodiment of respiratory condition inference engine 278 described in connection with FIG. 2. Determining that the user's respiratory condition has changed may be determining that the phoneme feature set distance satisfies a threshold distance (e.g., a condition-change threshold), which may be pre-determined by a caregiver or clinician or determined based on physiological data of the user (e.g., self-reported), a user setting, or historical respiratory-condition information for the user. Alternatively, the condition-change threshold may be pre-set based on reference population of monitored individuals.

In some embodiments, determining that the user's respiratory condition has changed may include determining whether the user's respiratory condition is getting better, getting worse, or not changing at all (e.g., not getting better or worse). This may include comparing the determined phoneme feature-set distance to a condition-change baseline, which may be a generic baseline determined from information on a reference population or may be determined for the user based on previous user data. For example, a third phoneme feature vector representing a healthy baseline may be determined from audio data captured at a time when the user was determined not to have a respiratory infection, and a second phoneme feature-set distance is determined by performing a second comparison between the second (i.e., most recent) and third (i.e., baseline) phoneme feature vectors. A third phoneme feature-set distance may also be determined by performing a third comparison between the first (i.e., earlier) and third (i.e., baseline) phoneme feature vectors. The third phoneme feature-set distance (representing a change between the healthy baseline and the first phoneme feature vector) is compared to the second phoneme feature set-distance (representing a change between the health baseline and the second phoneme feature vector from data captured subsequent to the first phoneme feature vector). If the second phoneme feature-set distance is less than the third feature-set distance (such that the vector from the most recently obtained data is closer to the healthy baseline), a user's respiratory condition may be determined to be improving. If the second phoneme feature-set distance is greater than the third feature-set distance (such that the vector from the most recently obtained data is further from the healthy baseline), a user's respiratory condition may be determined to be worsening. If the second phoneme feature-set distance is equal to the third feature-set distance, a user's respiratory condition may be determined to be not changing (or least not generally improving or worsening).

At step 6240, an action is initiated based on the determined change in the user's respiratory condition. Example actions may include actions and recommendations for treating the respiratory condition and/or symptoms of the condition. Step 6240 may be performed by embodiments of decision support tool(s) 290 (including sick monitor 292, prescription monitor 294 and/or medication efficacy tracker 296) and/or presentation component 220 in FIG. 2.

The action may include sending or otherwise electronically communicating an alert or a notification to a user via a user device, such as user devices 102a-n in FIG. 1, or to a clinician via a clinician user device, such as clinician user device 108 in FIG. 1. The notification may indicate whether or not there is a change in the user's respiratory condition and, in some embodiments, whether the change is an improvement or not. The notification or alert may include a respiratory-condition score quantifying or characterizing a change in the user's respiratory condition and/or a current state of the respiratory condition.

In some embodiments, an action may further include processing the respiratory condition information for decision-making, which may include providing a recommendation for treatment and support based on user's respiratory condition. Such a recommendation may include a recommendation to consult with a healthcare provider, continue an existing prescription or over-the-counter medicine (such as re-fill a prescription), modify the dosage and or medication of current treatment, and/or continue monitoring the respiratory condition. One or more of these actions within the recommendations may be performed in response to the detected change (or lack of change) in the respiratory condition. For example, an appointment with the user's healthcare provider may be scheduled and/or a prescription may be refilled by embodiments of this disclosure based on the determined change (or lack thereof).

FIGS. 7 through 14 depict various aspects of example embodiments of the disclosure actually reduced to practice. For instance, FIGS. 7 through 14 illustrate aspects of acoustic features analyzed, correlations between acoustic features and user's respiratory condition (including symptoms), and self-reported information. The information reflected in the figures may have been collected over a number of collection checkpoints (e.g., in a clinic/lab and/or at home) for multiple users. An example process of collecting the information is described in conjunction with FIG. 3B.

FIG. 7, in one embodiment, depicts representative changes in example acoustic features over time. In this embodiment, acoustic features are extracted from voice samples obtained in two collection checkpoints (visit 1 and visit 2). Visit 1 may represent a collection checkpoint during which the user is sick, while visit 2 may represent a collection checkpoint during which the user is well (i.e., has recovered from being sick). As shown in FIG. 7, features are measured for seven phoneme, and graphs 710, 720, and 730 depict changes in the acoustic features for each phoneme between the two visits. Graph 710 depicts changes in jitter (a measure of pitch instability); graph 720 depicts changes in shimmer (a measure of amplitude); and graph 730 depicts changes in spectral contrast. Graphs 710 and 720 show that jitter and shimmer decrease during recovery (i.e., between visit 1 and visit 2) for all phonemes, indicating that individuals may have better voice stability after recovery from a respiratory infection. Graph 730 shows that spectral contrast at higher frequencies increases for nasal sounds (/n/, /m/ and /ng/), which is consistent with nasal resonances being more pronounced as congestion reduces during recovery.

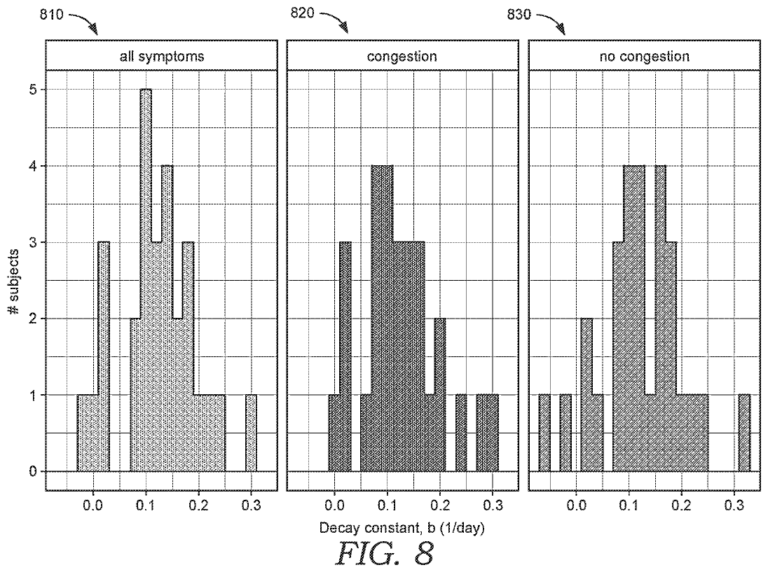

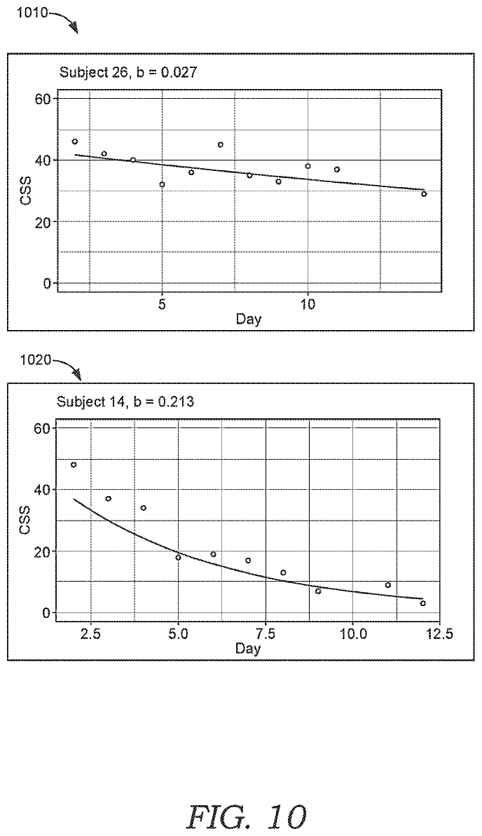

FIG. 8 depicts graphic representations of decay constants for respiratory infection symptoms. Histogram 810 shows decay constants for all symptoms, histogram 820 shows decay constants for congestion symptoms, and histogram 830 shows decay constants for non-congestion symptoms. Examples of congestion symptoms may include need to blow nose, nasal obstruction, and post-nasal discharge, while examples of non-congestion symptoms may include runny nose, cough, sore throat, and thick nasal discharge. The exponential decay model utilized for histograms 810, 820, and 830 is score ˜ae^{-b(day-1)}+ϵ, which is then fitted to the daily symptom phenotype (i.e., congestion, non-congestion, or all) for a group of monitored users. Positive values in histograms 810, 820, and 830 correspond to a decrease in symptoms; zero value corresponds to no change; and negative values correspond to a worsening of symptoms. Histograms 810, 820, and 830 show that recovery profiles of self-reported symptoms are variable. Two examples of recovery profiles are described in conjunction with FIG. 10.

FIG. 9 depicts correlations between acoustic features and self-reported respiratory infection symptoms. Graph 900 is based on separate decay constants that are computed for the sum of ratings for all symptoms (e.g., a composite symptom score), the sum of all congestion-related symptoms' ratings, and the sum of all non-congestion-related symptoms' ratings. Spearman correlation coefficients are computed, and all correlation values with a trend towards significance (p<0.1) are shown in graph 900 as a function of symptom group. Absolute values of correlation are plotted in graph 900.

For most acoustic features, the direction of correlation is the same between symptom groups. However, formant 1 bandwidth variability (bw1sdF) is positively correlated with non-congestion symptoms, but negatively correlated with congestion symptoms (and thus, uncorrelated with all summed symptoms). Graph 900 shows a stronger correlation between changes in higher-frequency spectral structure and changes in self-reported symptoms associated with the congestion phenotype compared to the non-congestion phenotype.

FIG. 10 depicts changes in self-reported symptom scores over time for two individuals. Graph 1010 depicts change for one individual (subject 26), which has a slow decay in composite symptom scores (CSS) during recovery. Graph 1020, by contrast, illustrate that another individual (subject 14) has a relatively fast decay in CSS during recovery.

FIGS. 11A-11B depict graphic representations of rank correlation between distance metric computed for different acoustic features and self-reported symptom scores. Graph 1100 in FIG. 11A represents rank correlations for a first set of acoustic features, whereas graph 1150 in FIG. 11B represents rank correlations for a second set of acoustic features. Graphs 1100 and 1150 show the distribution of Spearman's rank correlation between the distance metric for feature vectors and self-reported symptom scores (e.g., CSS) across a group of monitored individuals for every possible combination of seven phonemes (/a/, /e/, /u/, /ae/, /n/, /m/, and/or /ng/). The phoneme combinations are sorted in an ascending order based on the coefficient of quartile variation (IQR/median).

These acoustic features in graphs 1100 and 1150 may be extracted from voice samples collected on different days, in accordance with embodiments of the disclosure. One voice sample may be collected from each individual on a day that the individual is sick and another voice sample may be collected from each individual on a later day when the individual is well (i.e., not sick). Computation of the distance method may be done as described in conjunction with phoneme features comparer 274. The distance metrics are correlated (e.g., Spearman's r) against a score for the individual's self-reported symptoms, which may be determined as described in conjunction with self-reporting data evaluator 2746. Graphs 1100 and 1150 show that subsets that include phonemes /n/, /m/, and /a/ resulted in the lowest value of the coefficient of quartile variation, indicating a relevance to detect respiratory conditions. In one embodiment of the disclosure, based on the results shown in graphs 1100 and 1150, further down-selection may be performed using Sparse PCA to identify a subset of acoustic features for each of the three phonemes, and a subset of 32 total features (12 features from /n/, 12 features from /m/, and eight features from /a/) may be selected for making inferences and/or predictions about an individual's respiratory condition.

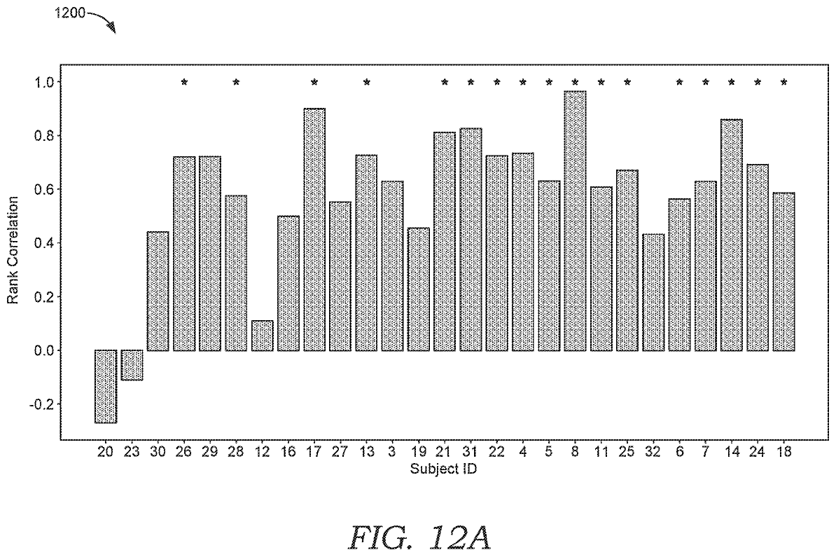

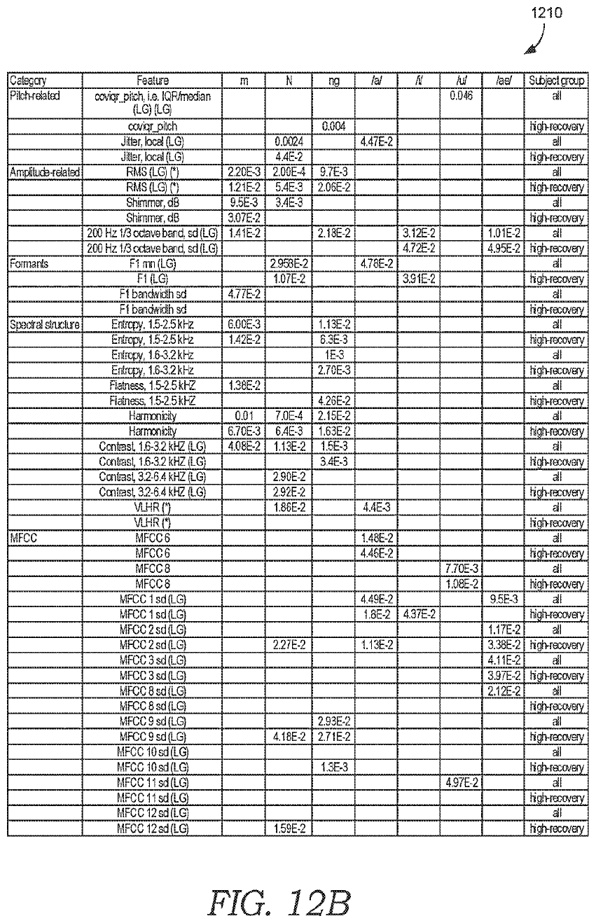

FIG. 12A depicts a graph 1200 showing rank correlation values between distance metrics and self-reported symptom scores across different individuals. The distance metrics utilized to compute rank correlation values may be based on 32 phoneme features derived from three phonemes (e.g., /n/, /m/, and /a/). Individuals are sorted left to right in graph 12200 in order of greatest change in symptoms (which may not necessarily correspond to the degree of rank correlation show by bars in graph 1200), and (*) indicates that a rank correlation shown is determined to be statistically significant (e.g., p<0.05). Graph 1200 illustrates that correlations are generally higher for individuals who exhibited a more rapid recovery (i.e., higher values of b). The average rank correlation for individuals with a b value higher than median is 0.7 (±0.13), compared to 0.46 (±0.33) for individuals with a b value lower than the median. The median correlation between the computed distance metric and self-reported composite symptom scores (CSS) is 0.63.

FIG. 12B depicts results of paired T-tests (p-values) for changes between sick and well visits to show statistically significant correlations in accordance with one embodiment of the disclosure. Only values where p<0.05 are included in table 1210. Table 1210 shows results for all individuals studied and for only individuals in the high-recovery group (as measured by decay constant b. In table 910, standard deviation is noted by “sd”, and log-transform is noted by “LG”.

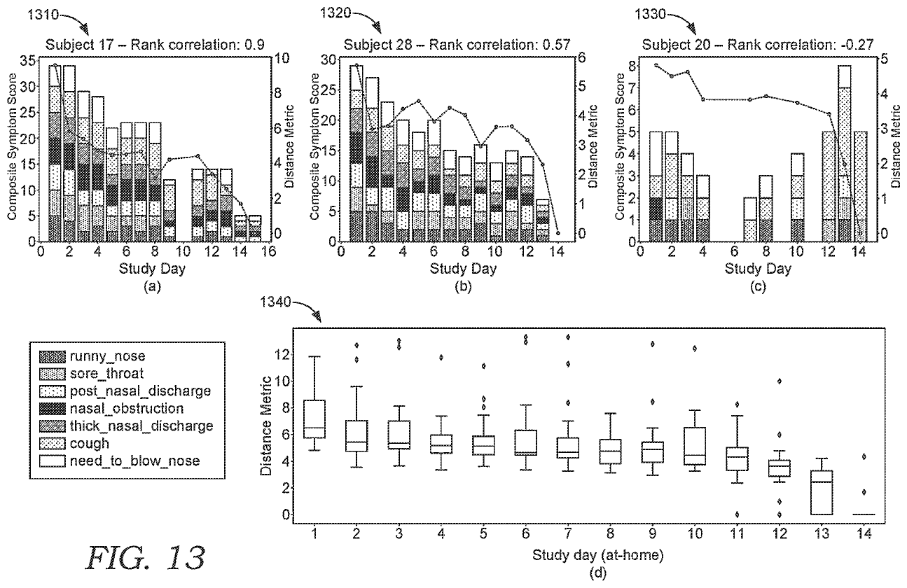

FIG. 13 depicts graphic representations of relative changes in acoustic features and self-reported symptoms over time for three example individuals identified as subjects 17, 20, and 28, in accordance with some embodiments Graphs 1310, 1320, and 1330 each depict changes in self-reported composite symptom scores (CSS) (denoted by vertical bars) and distance metrics computed from phoneme feature vectors (denoted by dashed line) over time for each individual. Graph 1310 illustrates that subject 17 showed a significant and relatively monotonic reduction in symptoms over time, which is reflected in the distance metric as well. Graph 1320 illustrates that the reduction in symptoms of subject 28 was more gradual and less monotonic compared to subject 17 and that the recovery of subject 28 stabilized around day 7-12 before a slight drop in symptoms on day 13. Graph 1320 also shows agreement with the distance metric is moderate and an observable transition from illness to recovery. In contrast to graphs 1310 and 1320, graph 1330 illustrates that the self-reported symptoms for subject 20 were mild (CSS=5 on day 1) to start with and non-congestion symptoms (cough and sore throat) worsened over time. Consequently, there is less agreement with the distance metric in graph 1330 relative to graphs 1310 and 1320.

Graph 1340 in FIG. 13 comprises a box plot of the computed distance metrics over time across a group of monitored individuals that include subjects 17, 20, and 28. Graph 1340 shows that distance tends to decrease as individuals near a recovered (or “well”) state, which may be around 14 days.

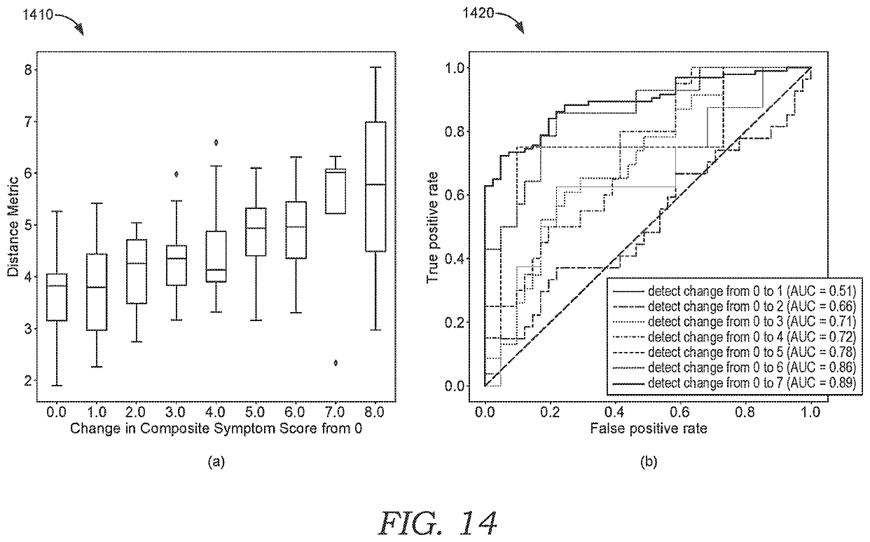

FIG. 14 depicts example representations of performance of a respiratory infection detector. Specifically, FIG. 14 illustrates a quantification of the ability of an embodiment of the disclosure to detect changes in respiratory condition, as measured by the self-reported symptom scores (e.g., CSS). Graph 1410 plots distance metric changes against changes in self-reported symptom scores, showing that, as the difference in self-reported symptoms on a given day increases, the distance between phoneme feature vectors also increases. Graph 1420 depicts receiver operating characteristic (ROC) curves and associated area under the curve (AUC) values for detecting changes of different magnitude in the self-reported symptom scores, utilizing phoneme features (and the distance computed between phoneme feature vectors), in accordance with embodiments of the disclosure. As depicted, the AUC value is 0.89 for a 7-point change (representing 20% of a composite symptom score range that is from 0 to 35).

FIGS. 15A-15M depict example embodiments of computer program routines for extracting phoneme features from voice data for tracking respiratory conditions, as described herein. As such, computer program routine in FIGS. 15A-15M may be utilized by user voice monitor 260, or one or more of its subcomponents. Additionally, computer program routine in FIGS. 15A-15M may be utilized to perform one or more aspects of method 6100 and 6200 of FIGS. 6A and 6B, respectively.

Accordingly, various aspects of technology directed to systems and methods for monitoring a user's respiratory condition are provided. It is understood that various features, sub-combinations, and modifications of the embodiments described herein are of utility and may be employed in other embodiments without reference to other features or sub-combinations. Moreover, the order and sequences of steps shown in the example methods or processes are not meant to limit the scope of the present disclosure in any way, and in fact, the steps may occur in a variety of different sequences within embodiments hereof. Such variations and combinations thereof are also contemplated to be within the scope of embodiments of this disclosure.

Having described various implementations, an exemplary computing environment suitable for implementing embodiments of the disclosure is now described. With reference to FIG. 16, an exemplary computing device is provided and referred to generally as a computing device 1700. The computing device 1700 is one example of a suitable computing environment and is not intended to suggest any limitation as to the scope of use or functionality of embodiments of the disclosure. Neither should the computing device 1700 be interpreted as having any dependency or requirement relating to any one or combination of components illustrated.

Embodiments of the disclosure may be described in the general context of computer code or machine-useable instructions, including computer-useable or computer-executable instructions, such as program modules, being executed by a computer or other machine, such as a personal data assistant, a smartphone, a tablet PC, or other handheld or wearable device, such as a smartwatch. Generally, program modules, including routines, programs, objects, components, data structures, and the like, refer to code that performs particular tasks or implements particular abstract data types. Embodiments of the disclosure may be practiced in a variety of system configurations, including handheld devices, consumer electronics, general-purpose computers, or specialty computing devices. Embodiments of the disclosure may also be practiced in distributed computing environments, where tasks are performed by remote-processing devices that are linked through a communications network. In a distributed computing environment, program modules may be located in both local and remote computer storage media including memory storage devices.

With reference to FIG. 16, computing device 1700 includes a bus 1710 that directly or indirectly couples various devices including a memory 1712, one or more processor(s) 1714, one or more presentation component(s) 1716, one or more input/output (I/O) port(s) 1718, one or more I/O components 1720, and an illustrative power supply 1722. Some embodiments of computing device 1700 may further include one or more radios 1724. Bus 1710 represents one or more busses (such as an address bus, a data bus, or a combination thereof). Although various blocks of FIG. 16 are shown with lines for the sake of clarity, in reality, these blocks represent logical, not necessarily actual, components. For example, one may consider a presentation component such as a display device to be an I/O component. Also, a processor may have a memory. FIG. 16 is merely illustrative of an exemplary computing device that can be used in connection with one or more embodiments of the present disclosure. Distinction is not made between such categories as “workstation,” “server,” “laptop,” or “handheld device,” as all are contemplated within the scope of FIG. 16 and with reference to “computing device.”

Computing device 1700 typically includes a variety of computer-readable media. Computer-readable media can be any available media that can be accessed by computing device 1700 and includes both volatile and nonvolatile, and removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules, or other data. Computer storage media includes, but is not limited to, Random-access memory (RAM), Read-only memory (ROM), electrically erasable programmable read-only memory (EEPROM), flash memory or other memory technology, Compact Disc Read-Only Memory (CD-ROM), digital versatile disks (DVDs) or other optical disk storage, magnetic cassettes, magnetic tape, magnetic disk storage or other magnetic storage devices, or any other medium, which can be used to store the desired information and can be accessed by computing device 1700. Computer storage media does not comprise signals per se. Communication media typically embodies computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media, such as a wired network or a direct-wired connection, and wireless media, such as acoustic, radio frequency (RF), infrared, and other wireless media. Combinations of any of the above should also be included within the scope of computer-readable media.

Memory 1712 includes computer storage media in the form of volatile and/or nonvolatile memory. The memory may be removable, non-removable, or a combination thereof. Exemplary hardware devices include for example solid-state memory, hard drives, and optical-disc drives. Computing device 1700 includes one or more processor(s) 1714 that reads data from various devices such as memory 1712 or I/O components 1720. Presentation component(s) 1716 presents data indications to a user or other device. Exemplary presentation component(s) 1716 may include a display device, a speaker, a printing component, a vibrating component, and the like.

The I/O port(s) 1718 allow computing device 1700 to be logically coupled to other devices, including I/O components 1720, some of which may be built in. Illustrative components include a microphone, a joystick, a game pad, a satellite dish, a scanner, a printer, or a wireless device. The I/O components 1720 may provide a natural user interface (NUI) that processes air gestures, voice, or other physiological inputs generated by a user. In some instances, inputs may be transmitted to an appropriate network element for further processing. An NUI may implement any combination of speech recognition, touch and stylus recognition, facial recognition, biometric recognition, gesture recognition (both on screen and adjacent to the screen), air gestures, head and eye tracking, and touch recognition associated with displays on the computing device 1700. The computing device 1700 may be equipped with depth cameras, such as stereoscopic camera systems, infrared camera systems, RGB camera systems, and combinations of these, for gesture detection and recognition. Additionally, the computing device 1700 may be equipped with accelerometers or gyroscopes that enable detection of motion. The output of the accelerometers or gyroscopes may be provided to the display of the computing device 1700 to render immersive augmented reality or virtual reality.

Some embodiments of computing device 1700 may include one or more radio(s) 1724 (or similar wireless communication components). The radio(s) 1724 transmits and receives radio or wireless communications. The computing device 1700 may be a wireless terminal adapted to receive communications and media over various wireless networks. Computing device 1700 may communicate via wireless protocols, such as code division multiple access (“CDMA”), global system for mobiles (“GSM”), time division multiple access (“TDMA”), or other wireless means, to communicate with other devices. The radio communications may be a short-range connection, a long-range connection, or a combination of both. Herein, “short” and “long” types of connections do not refer to the spatial relation between two devices. Instead, these connection types are generally referring to short range and long range as different categories, or types, of connections (i.e., a primary connection and a secondary connection). A short-range connection may include, by way of example and not limitation, a Wi-Fi® connection to a device (e.g., mobile hotspot) that provides access to a wireless communications network, such as a Wireless Local Area Network (WLAN) connection using the 802.11 protocol; a Bluetooth connection to another computing device is another example of a short-range connection; or a near-field communication. A long-range connection may include a connection using, by way of example and not limitation, one or more of CDMA, General Packet Radio Service (GPRS), GSM, TDMA, and 802.16 protocols.

Many different arrangements of the various components depicted, as well as components not shown, are possible without departing from the scope of the claims below. Embodiments of the disclosure have been described with the intent to be illustrative rather than restrictive. Alternative embodiments will become apparent to readers of this disclosure after and because of reading it. Alternative means of implementing the aforementioned can be completed without departing from the scope of the claims below. Certain features and sub-combinations are of utility and may be employed without reference to other features and sub-combinations and are contemplated within the scope of the claims.

What is claimed is:

1. A computerized system for monitoring a respiratory condition of a human subject, the system comprising: one or more processors; and computer memory having computer-executable instructions stored thereon for performing operations when executed by the one or more processors, the operations comprising: receiving first audio data comprising voice information of the human subject, determining a first phoneme feature set comprising at least one acoustic feature characterizing a first portion of the first audio data, the first portion including a first phoneme; monitoring the respiratory condition by comparing the first phoneme feature set to a second phoneme feature set determined from second audio data.

2. The computerized system of claim 1 further comprising an acoustic sensor configured to capture audio information.

3. The computerized system of claim 2, wherein the acoustic sensor is integrated into a smart speaker.

4. The computerized system of claim 1, wherein the first phoneme feature set comprises acoustic features characterizing at least one phenome that comprises /a/, /e/, /n/, or /m/.

5. The computerized system of claim 1, wherein the first phoneme feature set comprises acoustic features characterizing a first phoneme associated with the first portion of the first audio data, a second phoneme associated with a second portion of the first audio data, and a third phoneme associated with a third portion of the first audio data, wherein the first phoneme comprises /a/, the second phoneme comprises /n/, and the third phoneme comprises /m/.

6. The computerized system of claim 5, wherein: the acoustic features for the /a/ phoneme comprise at least one of: standard deviation of formant 1 (F1) bandwidth, pitch interquartile range, spectral entropy determined for 1.6 to 3.2 kilohertz (kHz) frequencies, jitter, standard deviation of mel-frequency cepstral coefficient MFCC9 and MFCC12, mean of mel-frequency cepstral coefficient MFCC6, and spectral contrast determined for 3.2 to 6.4 kHz frequencies, the acoustic features for the /n/ phoneme comprise at least one of: harmonicity, standard deviation of F1 bandwidth, pitch interquartile range, spectral entropy determined for 1.5 to 2.5 kHz and 1.6 to 3.2 kHz frequencies, spectral flatness determined for 1.5 to 2.5 kHz frequencies, standard deviation of mel-frequency cepstral coefficients MFCC1, MFCC2, MFCC3, and MFCC11, mean of mel-frequency cepstral coefficient MFCC8, and spectral contrast determined for 1.6 to 3.2 kHz frequencies, and the acoustic features for the /m/ phoneme comprise at least one of: harmonicity, standard deviation of F1 bandwidth, pitch interquartile range, spectral entropy determined for 1.5 to 2.5 kHz and 1.6 to 3.2 kHz frequencies, spectral flatness determined for 1.5 to 2.5 kHz frequencies, standard deviation of mel-frequency cepstral coefficients MFCC2 and MFCC10, mean of mel-frequency cepstral coefficient MFCC8, shimmer, spectral contrast determined for 3.2 to 6.4 kHz frequencies, and standard deviation of 200 hertz (Hz) third-octave band.

7. The computerized system of claim 1, wherein the operations further comprise: performing automatic speech recognition on the first portion of the first audio data to determine a first phoneme; and associating the first portion of the first audio data with the first phoneme.

8. The computerized system of claim 7, wherein performing automatic speech recognition comprises: determining a text corresponding to the first portion of the first audio data; and determining the first phoneme based on the text.

9. The computerized system of claim 1, wherein the first audio data is associated with a first time interval corresponding to a first date-time value and the second audio data is associated with a second time interval corresponding to a second date-time value, and wherein monitoring the respiratory condition of the human subject comprises: determining a feature distance measurement of at least a portion of features in the first and second phoneme feature sets; and based on the feature distance measurement, determining that the respiratory condition of the human subject has changed between the second date-time value and the first date-time value.

10. The computerized system of claim 9, wherein the second date-time value occurs between 18 and 36 hours after the first date-time value.

11. The computerized system of claim 1, wherein the operations further comprise: receiving a first physiological data for the human subject, the first physiological data being associated with a first time interval that is associated with the first audio data; and storing the physiological data in the record.

12. The computerized system of claim 1, wherein the first audio data is associated with a first time interval and wherein the operations further comprise determining first contextual data for the human subject, the first contextual data being associated with a first time interval and comprising at least one of physiological data about the human subject, information about a location of the human subject during the first time interval, or contextual information associated with the first time interval, wherein the first phoneme feature set is further determined based on the first contextual data.

13. The computerized system of claim 1, wherein the first phoneme feature set is determined from a plurality of other phoneme feature sets, each of the other phoneme feature sets being associated with a first date-time value occurring before a second time interval associated with the second audio data.

14. The computerized system of claim 1, wherein comparing the first phoneme feature set to the second phoneme feature set comprises determining a Euclidian or Levenshtein distance between at least a portion of the first phoneme feature set and at least a portion of the second phoneme feature set.

15. The computerized system of claim 1, wherein comparing the first phoneme feature set to the second phoneme feature set comprises performing a comparison between at least a first feature of the first phoneme feature set and a corresponding second feature of the second phoneme feature set.

16. The computerized system of claim 1, wherein monitoring the respirator condition of the human subject comprises: performing a comparison of the first phoneme feature set and the second phoneme feature set to determine a first feature-set distance; and determining that the respiratory condition of the human subject has changed by comparing the first feature-set distance to a threshold distance.

17. The computerized system of claim 16, wherein the threshold distance is pre-determined by a clinician or is automatically determined based on one or more of: physiological data of the user, a user setting, or historical respiratory-condition information of the user.

18. The computerized system of claim 16, wherein the operations further comprise: receiving a third phoneme feature set representing a baseline at a time when the human subject is determined to not have the respiratory condition; and wherein monitoring the respirator condition of the human subject comprises: performing a comparison of the first phoneme feature set and the second phoneme feature set to determine a first feature-set distance; performing a second comparison between the second phoneme feature set and the third phoneme feature set to determine a second feature-set distance; perform a third comparison between the first phoneme feature set and the third phoneme feature set to determine a third feature-set distance; perform a fourth comparison of the second feature-set distance and the third feature-set distance; and based on the fourth comparison, perform one of: providing an indication that the human subject's respiratory condition is improving if the second feature-set distance is less than the third feature-set distance, providing an indication that the human subject's respiratory condition is worsening if the second feature-set distance is greater than the third feature-set distance or providing an indication that the human subject's respiratory condition is not changing if the second feature-set distance equals the third feature-set distance.

19. The computerized system of claim 2, wherein the third phoneme feature set representing the baseline comprises phoneme features having feature values determined based on an average of a set of phoneme feature values, each phoneme feature value within the set of phoneme feature values determined from a different time interval during the time when the human subject is determined to not have the respiratory condition.

20. The computerized system of claim 1, wherein the operations further comprise initiating an action based on a change in the respiratory condition determined by comparing the first phoneme feature set to the second phoneme feature set.

21. The computerized system of claim 20, wherein initiating an action based on the change in the respiratory condition of the human subject comprises issuing a notification to at least one of: a user device associated with the human subject or a clinician of the human subject; scheduling an appointment between the human subject and the clinician of the human subject; providing a recommendation to modify treatment of the respiratory condition; and requesting a prescription medication refill.

22. The computerized system of claim 1 further comprising a user device associated with the human subject, wherein monitoring the respiratory condition of the human subject comprises determining a respiratory condition-score based at least on comparing the first phoneme feature set to the second phoneme feature set, and wherein the operations further comprise causing for display, on a user interface of the user device, the respiratory condition score.

23. The computerized system of claim 1 further comprising a user device associated with the human subject, wherein monitoring the respiratory condition of the human subject comprises determining a transmission risk level indicating a risk of the human subject transmitting an infectious agent associated with the respiratory condition based at least on comparing the first phoneme feature set to the second phoneme feature set, and wherein the operations further comprise causing for display, on a user interface of the user device, the transmission risk level.

24. The computerized system of claim 1 further comprising a user device associated with the human subject, wherein monitoring the respiratory condition of the human subject comprises determining a trend in the respiratory condition of the human subject based at least on comparing the first phoneme feature set to the second phoneme feature set, and wherein the operations further comprise causing for display, on a user interface of the user device, the trend in the respiratory condition of the human subject.

25. The computerized system of claim 1, wherein the first portion of the first audio data comprises a sustained phonation of a cardinal vowel phoneme and wherein the first phoneme feature set is based on a maximum phonation time.

26. The computerized system of claim 1, wherein the first audio data comprises a recording of a spoken passage that includes multiple phonemes and wherein the first phoneme feature set comprises one or more of a speaking rate, an average pause length, a pause count, and a global signal-to-noise ratio.

27. A method for treating a respiratory condition utilizing an acoustic sensor device, the method comprising: receiving first audio data that is associated with a first time interval, the first audio data comprises voice information of a human subject; determining a first phoneme feature set comprising at least one acoustic feature characterizing a first portion of the first audio data, the first portion including a first phoneme; performing a comparison of the first phoneme feature set to a second phoneme feature set determined from second audio data associated with a second time interval; and based on at least the comparison, initiating a treatment protocol for the human subject to treat the respiratory condition.

28. The method of claim 27, wherein initiating the treatment protocol includes determining at least one of a therapeutic agent, a dosage, and a method of administration of the therapeutic agent.

29. The method of claim 28, wherein the therapeutic agent is selected from a group consisting of: a PLpro inhibitor, Apilomod, EIDD-2801, Ribavirin, Valganciclovir, β-Thymidine, Aspartame, Oxprenolol, Doxycycline, Acetophenazine, Iopromide, Riboflavin, Reproterol, 2,2′-Cyclocytidine, Chloramphenicol, Chlorphenesin carbamate, Levodropropizine, Cefamandole, Floxuridine, Tigecycline, Pemetrexed, L(+)-Ascorbic acid, Glutathione, Hesperetin, Ademetionine, Masoprocol, Isotretinoin, Dantrolene, Sulfasalazine Anti-bacterial, Silybin, Nicardipine, Sildenafil, Platycodin, Chrysin, Neohesperidin, Baicalin, Sugetriol-3,9-diacetate, (−)-Epigallocatechin gallate, Phaitanthrin D, Dihydroxyphenyl)-2-[[2-(3,4-dihydroxyphenyl)-3,4-dihydro-5,7-dihydroxy-2H-1-benzopyran-3-yl]oxy]-3,4-dihydro-2H-1-benzopyran-3,4,5,7-tetrol, 2,2-di(3-indolyl)-3-indolone, (S)-(1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl-2-amino-3-phenylpropanoate, Piceatannol, Rosmarinic acid, and Magnolol; a 3CLpro inhibitor, Lymecycline, Chlorhexidine, Alfuzosin, Cilastatin, Famotidine, Almitrine, Progabide, Nepafenac, Carvedilol, Amprenavir, Tigecycline, Montelukast, Carminic acid, Mimosine, Flavin, Lutein, Cefpiramide, Phenethicillin, Candoxatril, Nicardipine, Estradiol valerate, Pioglitazone, Conivaptan, Telmisartan, Doxycycline, Oxytetracycline, (1S,2R,4aS,5R,8aS)-1-Formamido-1,4a-dimethyl-6-methylene-5-((E)-2-(2-oxo-2,5-dihydrofuran-3-yl)ethenyedecahydronaphthalen-2-yl5-((R)-1,2-dithiolan-3-yl) pentanoate, Betulonal, Chrysin-7-O-β-glucuronide, Andrographiside, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl 2-nitrobenzoate, 2β-Hydroxy-3,4-seco-friedelolactone-27-oic acid (S)-(1S,2R,4aS,5R,8aS)-1-Formamido-1,4a-dimethyl-6-methylene-5-((E)-2-(2-oxo-2,5-dihydrofuran-3-yl)ethenyl) decahydronaphthalen-2-yl-2-amino-3-phenylpropanoate, Isodecortinol, Cerevisterol, Hesperidin, Neohesperidin, Andrograpanin, 2-((1R,5R,6R,8aS)-6-Hydroxy-5-(hydroxymethyl)-5,8a-dimethyl-2-methylenedecahydronaphthalen-1-yl)ethyl benzoate, Cosmosiin, Cleistocaltone A, 2,2-Di(3-indolyl)-3-indolone, Biorobin, Gnidicin, Phyllaemblinol, Theaflavin 3,3′-di-O-gallate, Rosmarinic acid, Kouitchenside I, Oleanolic acid, Stigmast-5-en-3-ol, Deacetylcentapicrin, and Berchemol; an RdRp inhibitor, Valganciclovir, Chlorhexidine, Ceftibuten, Fenoterol, Fludarabine, Itraconazole, Cefuroxime, Atovaquone, Chenodeoxycholic acid, Cromolyn, Pancuronium bromide, Cortisone, Tibolone, Novobiocin, Silybin, Idarubicin Bromocriptine, Diphenoxylate, Benzylpenicilloyl G, Dabigatran etexilate, Betulonal, Gnidicin, 213,3013-Dihydroxy-3,4-seco-friedelolactone-27-lactone, 14-Deoxy-11,12-didehydroandrographolide, Gniditrin, Theaflavin 3,3′-di-O-gallate, (R)-((1R,5aS,6R,9aS)-1,5a-Dimethyl-7-methylene-3-oxo-6-((E)-2-(2-oxo-2,5-dihydrofuran-3-yl)ethenyedecahydro-1H-benzo[c]azepin-1-yl)methyl2-amino-3-phenylpropanoate, 2β-Hydroxy-3,4-seco-friedelolactone-27-oic acid, 2-(3,4-Dihydroxyphenyl)-2-[[2-(3,4-dihydroxyphenyl)-3,4-dihydro-5,7-dihydroxy-2H-1-benzopyran-3-yl]oxy]-3,4-dihydro-2H-1-benzopyran-3,4,5,7-tetrol, Phyllaemblicin B, 14-hydroxycyperotundone, Andrographiside, 2-((1R,5R,6R,8aS)-6-Hydroxy-5-(hydroxymethyl)-5,8a-dimethyl-2-methylenedecahydro naphthalen-1-yl)ethyl benzoate, Andrographolide, Sugetriol-3,9-diacetate, Baicalin, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl 5-((R)-1,2-dithiolan-3-yl)pentanoate, 1,7-Dihydroxy-3-methoxyxanthone, 1,2,6-Trimethoxy-8-1(6-O-β-D-xylopyranosyl-(3-D-glucopyranosyl)oxy]-9H-xanthen-9-one, and/or 1,8-Dihydroxy-[(6-methoxy-2-[(6-O-β-D-xylopyranosyl-β-D-glucopyranosyl)oxy]-9H-xanthen-9-one, 8-(β-D-Glucopyranosyloxy)-1,3,5-trihydroxy-9H-xanthen-9-one; Diosmin, Hesperidin, MK-3207, Venetoclax, Dihydroergocristine, Bolazine, R428, Ditercalinium, Etoposide, Teniposide, UK-432097, Irinotecan, Lumacaftor, Velpatasvir, Eluxadoline, Ledipasvir, a combination of Lopinavir/Ritonavir and Ribavirin, Alferon, and prednisone; dexamethasone, azithromycin, remdesivir, boceprevir, umifenovir and favipiravir; an α-ketoamides compound; an RIG 1 pathway activator; a protease inhibitor; and remdesivir, galidesivir, favilavir/avifavir, molnupiravir (MK-4482/EIDD 2801), AT-527, AT-301, BLD-2660, favipiravir, camostat, SLV213 emtrictabine/tenofivir, clevudine, dalcetrapib, boceprevir, ABX464, (3S)-3-({N-[(4-methoxy-1H-indol-2-yl)carbonyl]-L-leucyl}amino)-2-oxo-4-[(3S)-2-oxopyrrolidin-3-yl]butyl dihydrogen phosphate; and a pharmaceutically acceptable salt, solvate or hydrate thereof (PF-07304814), (1R,2S,5S)—N-{(1S)-1-Cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide or a solvate or hydrate thereof (PF-07321332), S-217622, glucocorticoids, convalescent plasma, a recombinant human plasma, monoclonal antibody, ravulizumab, VIR-7831/VIR-7832, BRII-196/BRII-198, COVI-AMG/COVI DROPS (STI-2020), bamlanivimab (LY-CoV555), mavrilimab, leronlimab (PRO140), AZD7442, lenzilumab, infliximab, adalimumab, JS 016, STI-1499 (COVIGUARD), lanadelumab (Takhzyro), canakinumab (Ilaris), gimsilumab, otilimab, antibody cocktail, recombinant fusion protein, anticoagulant, IL-6 receptor agonist, PlKfyve inhibitor, RIPK1 inhibitor, VIP receptor agonist, SGLT2 inhibitor, TYK inhibitor, kinase inhibitor, bemcentinib, acalabrutinib, losmapimod, baricitinib, tofacitinib, H2 blocker, anthelmintic, and a furin inhibitor.

30. The method of claim 28, wherein the therapeutic agent is (3S)-3-({N-[(4-methoxy-1H-indol-2-yl)carbonyl]-L-leucyl}amino)-2-oxo-4-[(3S)-2-oxopyrrolidin-3-yl]butyl dihydrogen phosphate, or a pharmaceutically acceptable salt, solvate or hydrate thereof (PF-07304814).

31. The method of claim 38, wherein the therapeutic agent is (1R,2S,5S)—N-{(1S)-1-Cyano-2-[(3S)-2-oxopyrrolidin-3-yl]ethyl}-6,6-dimethyl-3-[3-methyl-N-(trifluoroacetyl)-L-valyl]-3-azabicyclo[3.1.0]hexane-2-carboxamide or a solvate or hydrate thereof (PF-07321332).

32. The method of claim 27, wherein initiating administration of the treatment protocol includes generating a graphic user interface element provided for display on a user device, the graphic user interface element indicating a recommendation of the treatment protocol that is based on at least the comparison of the first phoneme feature set to the second phoneme feature set.

33. The method of claim 32, wherein the user device is separate from the acoustic sensor device.

34. The method of claim 32 further comprising applying the treatment protocol to the human subject based on the recommendation.

35. The method of claim 27, wherein the respiratory condition comprises coronavirus disease 2019 (COVID-19).

36. A computerized method of tracking efficacy of a therapeutic agent for treating a respiratory condition in a human subject, the computerized method comprising: receiving a first phoneme feature set and a second phoneme feature set, each of the first phoneme feature set and the second phoneme feature set representing voice information of the human subject, the second phoneme feature set being associated with a second date-time value occurring after a first date-time value associated with the first phoneme feature set, wherein a time period in which the therapeutic agent is being administered to the human subject includes at least the second date-time value; performing a first comparison of the first phoneme feature set and the second phoneme feature set to determine a first feature-set distance; and based on the first feature-set distance, determining whether there is a change in the respiratory condition of the human subject.

37. The computerized method of claim 36, wherein the respiratory condition is a respiratory infection, and wherein the therapeutic agent is an antimicrobial medication.

38. The computerized method of claim 37, wherein the therapeutic agent is an antibiotic medication.

39. The computerized method of claim 37 further comprising, based at least on determining whether there is a change in the respiratory condition of the human subject, determining a change in efficacy of the antibiotic medication.

40. The computerized method of claim 36, wherein determining whether there is a change in the respiratory condition of the human subject comprises determining whether the respiratory condition has improved, worsened, or not changed.

41. The computerized method of claim 36 further comprising: based on the determination of whether there is a change in the respiratory condition of the human subject, initiating an action for treating the human subject.

42. The computerized method of claim 41, wherein the action for treating the human subject is initiated upon determining that the respiratory condition has worsened.

43. The computerized method of claim 41, wherein the action for treating the human subject is initiated upon determining that the respiratory condition has either worsened or not changed.

44. The computerized method of claim 41, wherein the action for treating the human subject comprising changing a treatment protocol of the human subject.

45. The computerized method of claim 44, wherein changing the treatment protocol of the human subject comprises initiating a recommendation to adjust one or more of the therapeutic agent or dosage of the therapeutic agent.

46. The computerized method of claim 44, wherein changing the treatment protocol of the human subject comprises sending a message to a care provider of the human subject, the message requesting a modification of the treatment protocol of the human subject.

47. The computerized method of claim 41, wherein the action for treating the human subject comprising electronically initiating a refill request for the therapeutic agent with a pharmacy determined from an electronic health record (EHR) of the human subject.
